SEQUENCE LISTING (92 sequences)

SEQ ID NO: 1 - protein, 8 aa, homo sapiens
IEPRSFSQ

SEQ ID NO: 2 - protein, 8 aa, homo sapiens
LDRRGIQR

SEQ ID NO: 3 - protein, 11 aa, homo sapiens
AELQGRPISIA

SEQ ID NO: 4 - protein, 10 aa, homo sapiens
TEGEARGSVI

SEQ ID NO: 5 - protein, 10 aa, homo sapiens
TESESRGAIY

SEQ ID NO: 6 - protein, 9 aa, homo sapiens
WEVAHQKLA

SEQ ID NO: 7 - protein, 8 aa, homo sapiens
HQKLVFFA

SEQ ID NO: 8 - protein, 12 aa, homo sapiens
SPLAQAVRSSSR

SEQ ID NO: 9 - protein, 8 aa, homo sapiens
LAQFVRSS

SEQ ID NO: 10 - protein, 9 aa, homo sapiens
HGDQMAQKS

SEQ ID NO: 11 - protein, 7 aa, homo sapiens
  PEPTIDE (5)..(5): “X” is homo-F
LAQSXRS

SEQ ID NO: 12 - protein, 9 aa, homo sapiens
HGDQMAQKS

SEQ ID NO: 13 - protein, 8 aa, homo sapiens
AVRSSSRG

SEQ ID NO: 14 - protein, 7 aa, homo sapiens
YVADAPK

SEQ ID NO: 15 - protein, 8 aa, homo sapiens
TSGPNQEQ

SEQ ID NO: 16 - protein, 9 aa, homo sapiens
DLTTYGFGH

SEQ ID NO: 17 - protein, 9 aa, homo sapiens
YEVHHQKLV

SEQ ID NO: 18 - protein, 10 aa, homo sapiens
SEVNLDAEFR

SEQ ID NO: 19 - protein, 5 aa, homo sapiens
LPETG

SEQ ID NO: 20 - protein, 7 aa, homo sapiens
PLLAERP

SEQ ID NO: 21 - protein, 11 aa, homo sapiens
TPLKSPPPSPR

SEQ ID NO: 22 - protein, 7 aa, homo sapiens
YVADAPV

SEQ ID NO: 23 - protein, 8 aa, homo sapiens
ISVDRPVK

SEQ ID NO: 24 - protein, 6 aa, homo sapiens
GFARGG

SEQ ID NO: 25 - protein, 6 aa, homo sapiens
GFLRGG

SEQ ID NO: 26 - protein, 8 aa, homo sapiens
LESDYFGK

SEQ ID NO: 27 - protein, 8 aa, homo sapiens
LESDYFGK

SEQ ID NO: 28 - protein, 8 aa, homo sapiens
PFDLLDFN

SEQ ID NO: 29 - protein, 8 aa, homo sapiens
GDEVDGAK

SEQ ID NO: 30 - protein, 8 aa, homo sapiens
NDTDANPR

SEQ ID NO: 31 - protein, 8 aa, homo sapiens
IQADSGPI

SEQ ID NO: 32 - protein, 8 aa, homo sapiens
LQLDCVAV

SEQ ID NO: 33 - protein, 6 aa, homo sapiens
LLVYGG

SEQ ID NO: 34 - protein, 6 aa, homo sapiens
YDAKGD

SEQ ID NO: 35 - protein, 9 aa, homo sapiens
GKPILFFRL

SEQ ID NO: 36 - protein, 9 aa, homo sapiens
GKPIIFFRL

SEQ ID NO: 37 - protein, 6 aa, homo sapiens
AAFFAA

SEQ ID NO: 38 - protein, 9 aa, homo sapiens
GKPILFFRL

SEQ ID NO: 39 - protein, 6 aa, homo sapiens
  PEPTIDE (4)..(4): “X” is Gnf
FVTXSW

SEQ ID NO: 40 - protein, 8 aa, homo sapiens
DTDLYDYY

SEQ ID NO: 41 - protein, 6 aa, homo sapiens
HPGGPQ

SEQ ID NO: 42 - protein, 10 aa, homo sapiens
EKARVLAEAA

SEQ ID NO: 43 - protein, 10 aa, homo sapiens
EKARVLAEAA

SEQ ID NO: 44 - protein, 4 aa, homo sapiens
APVK

SEQ ID NO: 45 - protein, 8 aa, homo sapiens
LAYGLRSK

SEQ ID NO: 46 - protein, 9 aa, homo sapiens
SLGRKIQIQ

SEQ ID NO: 47 - protein, 10 aa, homo sapiens
RGVVNASSRL

SEQ ID NO: 48 - protein, 8 aa, homo sapiens
RPPGFSAF

SEQ ID NO: 49 - protein, 6 aa, homo sapiens
GRGRGG

SEQ ID NO: 50 - protein, 8 aa, homo sapiens
FNPRTFGS

SEQ ID NO: 51 - protein, 8 aa, homo sapiens
RQKRFVLS

SEQ ID NO: 52 - protein, 8 aa, homo sapiens
RKRRSVNP

SEQ ID NO: 53 - protein, 8 aa, homo sapiens
YGPKKKRK

SEQ ID NO: 54 - protein, 8 aa, homo sapiens
  PEPTIDE (6)..(6): “X” is Abu
DDMEEXAS

SEQ ID NO: 55 - protein, 10 aa, homo sapiens
EDVVACSMSY

SEQ ID NO: 56 - protein, 9 aa, homo sapiens
VEDVVCCSM

SEQ ID NO: 57 - protein, 8 aa, homo sapiens
SQNYPIVQ

SEQ ID NO: 58 - protein, 8 aa, homo sapiens
PPEELKFQ

SEQ ID NO: 59 - protein, 8 aa, homo sapiens
AIFQGPID

SEQ ID NO: 60 - protein, 10 aa, homo sapiens
GFSPFRASRV

SEQ ID NO: 61 - protein, 6 aa, homo sapiens
GKAFRR

SEQ ID NO: 62 - protein, 6 aa, homo sapiens
AFRFSQ

SEQ ID NO: 63 - protein, 10 aa, homo sapiens
GVGVTETPLM

SEQ ID NO: 64 - protein, 15 aa, homo sapiens
RRKKVYPYPMEGTIA

SEQ ID NO: 65 - protein, 8 aa, homo sapiens
PTLQLPLA

SEQ ID NO: 66 - protein, 8 aa, homo sapiens
  PEPTIDE (3)..(3): “X” is Nle
ERXFLSFP

SEQ ID NO: 67 - protein, 8 aa, homo sapiens
ERMFLSFP

SEQ ID NO: 68 - protein, 10 aa, homo sapiens
ALERMFLSFP

SEQ ID NO: 69 - protein, 8 aa, homo sapiens
  PEPTIDE (4)..(6): “X1” is Cha, “X2” is Abu, “X3” is Smc
KGPXXXHA

SEQ ID NO: 70 - protein, 8 aa, homo sapiens
GSHLVEAL

SEQ ID NO: 71 - protein, 8 aa, homo sapiens
PKAKSHAP

SEQ ID NO: 72 - protein, 7 aa, homo sapiens
PQFRIKG

SEQ ID NO: 73 - protein, 10 aa, homo sapiens
ALERMFLSFP

SEQ ID NO: 74 - protein, 7 aa, homo sapiens
VADVKDR

SEQ ID NO: 75 - protein, 7 aa, homo sapiens
  PEPTIDE (1)..(1): “X” is L2p
XYDAKGD

SEQ ID NO: 76 - protein, 8 aa, homo sapiens
APAKFFRL

SEQ ID NO: 77 - protein, 10 aa, homo sapiens
STPVSSANMK

SEQ ID NO: 78 - protein, 9 aa, homo sapiens
EDAEPYAAK

SEQ ID NO: 79 - protein, 10 aa, homo sapiens
IHPFHLVIHT

SEQ ID NO: 80 - protein, 7 aa, homo sapiens
SVTLQSG

SEQ ID NO: 81 - protein, 10 aa, homo sapiens
TSAVLQSGFR

SEQ ID NO: 82 - protein, 12 aa, homo sapiens
FRLKGGAPIKGV

SEQ ID NO: 83 - protein, 12 aa, homo sapiens
SPLAQAVRSSSR

SEQ ID NO: 84 - protein, 9 aa, homo sapiens
DYVPRGSKD

SEQ ID NO: 85 - protein, 8 aa, homo sapiens
IEPRSFSQ

SEQ ID NO: 86 - protein, 8 aa, homo sapiens
GVPRSFRG

SEQ ID NO: 87 - protein, 10 aa, homo sapiens
ENLYFQSGTK

SEQ ID NO: 88 - protein, 8 aa, homo sapiens
LYFQSGTV

SEQ ID NO: 89 - protein, 8 aa, homo sapiens
GRGHARLV

SEQ ID NO: 90 - protein, 8 aa, homo sapiens
GHARLVHG

SEQ ID NO: 91 - protein, 8 aa, homo sapiens
GPAKLAIG

SEQ ID NO: 92 - protein, 11 aa, homo sapiens
FASGKRSQIGL

(19) United States
(12) Patent Application Publication
(10) Pub. No.: US 2010/0279328 A1
(43) Pub. Date: Nov. 4, 2010

(54) Fret assays, methods for performing the assays and compounds relevant to the assays

(76) Inventors: Anita Hong, Cupertino, CA (US); Vera Rakhmanova, Foster City, CA (US); Xiao-He Tong, Mountain View, CA (US)

Correspondence Address: Jeffrey A. McKinney;McKinney Law Group APC, 851 Moraga Road, Lafayette, CA, 94549, US

(21) Appl. No.: 12/658,447

(22) Filed: Feb. 5, 2010

Related U.S. Application Data
(60) Provisional application No. 61/207,006, filed on Feb. 6, 2009.

(51) Int. Cl. C12Q 1/34; C12N 9/48

(52) U.S. Cl. 435/18; 435/212

(57) ABSTRACT

The present invention is generally directed to biological assays. More specifically it is directed to FRET-based assays using particularly effecting FRET pairs, methods for performing such assays and the molecules utilized in the assays. In a composition aspect, the present invention is directed to a FRET-based protease substrate selected from the list of substrates shown in the Detailed Description section above. In a method aspect, the present invention is directed to a method of performing a FRET-based assay, where the assay includes the following steps: adding a solution or suspension containing one or more different proteins to a reaction vessel; adding a liquid comprising a FRET-substrate to the reaction vessel, wherein the FRET-substrate is selected from the list of substrates shown in the Detailed Description section above. In a kit aspect, the present invention is directed to a kit for performing a FRET-based assay, wherein the kit comprises a protease substrate. The protease substrate is selected from the list of substrates shown in the Detailed Description section above.

This application claims priority to U.S. Provisional Patent Appl. No. 61/207,006 under 35 U.S.C. §119. Provisional application 61/207,006 is incorporated-by-reference into this document for all purposes.

### FIELD OF THE INVENTION

The present invention is generally directed to biological assays. More specifically it is directed to FRET-based assays using particularly effecting FRET pairs, methods for performing such assays and the molecules utilized in the assays.

### BACKGROUND OF THE INVENTION

Proteases are enzymes that cleave protein substrates to form smaller proteins. The smaller proteins exhibit specific beneficial or detrimental functions in the host organism. Where detrimental functions are exhibited, the proteins oftentimes are the root cause of various diseases. Examples of such diseases include cancer, arthritis, Alzheimer's, hypertension, hepatitis and AIDS.

Pharmaceutical research directed to proteases came to the scientific forefront when researchers realized that an HIV protease could be the key to treating early-stage HIV infections. When the research uncovered several small molecule inhibitors of HIV protease, and the inhibitors proved effective in a clinical setting, discovering new proteases, understanding their function, and using them in the development of novel pharmaceuticals became an industry priority.

The development of protease-based pharmaceuticals required the emergence of new assays that one could use to monitor the effect of small or large molecules on protease activity. Preferred assays were homogeneous, meaning one could obtain a continuous reading of activity rather than stopping enzymatic activity, separating reaction components and then obtaining an activity reading. Useful assays furthermore had to be very sensitive so one could measure activity even when only a small amount of protease was present in the reaction medium.

Given the pharmaceutical companies' assay requirements, spectrophotometric-based assays appeared to be the likely answer for probing protease activity; they are homogeneous, fast, accurate and easy to use. Spectrophotometric assays are either fluorogenic or chromogenic, with fluorogenic assays being superior in many cases. This is because the fluorogenic assays have a wider linear dynamic range and offer better reproduceability. Förster resonance energy transfer (“FRET”) assays are a type of fluorogenic spectrophotometric assay pharmaceutical companies have oftentimes used for rapid activity screening.

FRET involves the transfer of excited-state energy from one type of compound (“donor”) to another type (“acceptor”). The donor typically transfers (i.e., emits) short wavelengths of light that are within the wavelength range the acceptor can absorb. Energy transfer occurs within a specified distance between the donor and acceptor, usually 10 to 100 Å. The distance at which energy transfer equals 50 percent is called the Förster radius. Within this distance, a decrease in donor fluorescence can be detected upon donor to acceptor energy transfer.

The use of FRET in a protease assay involves the construction of a protease substrate that includes both a donor and an acceptor positioned on opposite sides of the cleavage site at an appropriate distance from one another. The substrate is typically constructed from a peptide sequence derived from a known protein cleaved by the protease. Spectrophotometric methods allow one to observe whether the protease substrate has been cleaved, since cleavage provides that the donor-acceptance distance exceeds the Forster radius; this results in recover of the donor's fluorescence.

Reports that discuss the use of FRET-based assays for examination of protease activity include, for example: U.S. Pat. No. 6,291,201 assigned to Zeneca Limited, entitled, “Fluorescence Energy Transfer Substrates”; U.S. Pat. No. 6,445,268 assigned to Iconix Pharmaceuticals Ltd., entitled, “Hydrolytic Enzyme Substrates and Assay Method”; U.S. Pat. No. 7,157,553 assigned to the United States, entitled “Highthroughput Assays for the Proteolytic Activities of Clostridial Neurotoxins.” Each of the preceding patents is incorporated-by-reference for all purposes into this document.

Despite the substantial work performed to date on FRET-based assays, there is still a need for more and different assays, methods to perform the assays and compounds relevant to the assays.

### SUMMARY OF THE INVENTION

The present invention is generally directed to biological assays. More specifically it is directed to FRET-based assays using particularly effecting FRET pairs, methods for performing such assays and the molecules utilized in the assays.

In a composition aspect, the present invention is directed to a FRET-based protease substrate selected from the list of substrates shown in the Detailed Description section below.

In a method aspect, the present invention is directed to a method of performing a FRET-based assay, where the assay includes the following steps: adding a solution or suspension containing one or more different proteins to a reaction vessel; adding a liquid comprising a FRET-substrate to the reaction vessel, wherein the FRET-substrate is selected from the list of substrates shown in the Detailed Description section below.

In a kit aspect, the present invention is directed to a kit for performing a FRET-based assay, wherein the kit comprises a protease substrate. The protease substrate is selected from the list of substrates shown in the Detailed Description section below.

### BRIEF DESCRIPTION OF THE DRAWINGS

FIG. 1 shows the structure of 6-FAM designated as “A1” and FITC designated as “A2”.

FIG. 2 shows the structure of Alexa Fluor 488 designated as “A3”.

FIG. 3 shows the structure of two compounds that are acceptor moieties in a FRET pair according to the present invention. The compounds are respectively designated as “B1” and “B2”.

FIG. 4 shows the structure of two compounds that are acceptor moieties in a FRET pair according to the present invention. The compounds are respectively designated as “B3” and “B4”.

FIG. 5 shows the structure of two compounds that are acceptor moieties in a FRET pair according to the present invention. The compounds are respectively designated as “B5” and “B6”.

FIG. 6 shows the structure of two compounds that are acceptor moieties in a FRET pair according to the present invention. The compounds are respectively designated as “B7” and “B8”.

FIG. 7 shows the structure of two compounds that are acceptor moieties in a FRET pair according to the present invention. The compounds are respectively designated as “B9” and “B10”.

### DETAILED DESCRIPTION OF THE INVENTION

With extensive experience, the inventors have developed a set of guidelines that aid in the development of a FRET-based protease substrate. A donor/acceptor pair for inclusion within the substrate should be chosen such that the absorption spectrum of the acceptor overlaps with the emission spectrum of the donor. Generally, a fluorescent donor and a non-fluorescent acceptor are used to make protease peptide substrates. One should keep in mind, however, that the donor/acceptor pair choice may be limited by the fluorometer filter at hand.

The donor and acceptor molecules must be in close proximity (e.g., 10 to 100 Å) in order for the acceptor to effectively quench donor fluorescence. Once an active protease recognizes and cleaves the substrate into two separate fragments, the increase in the donor-acceptor distance causes FRET efficiency to decrease, resulting in the recovery of the donor's fluorescence. The time-dependent increase in fluorescence intensity is related to the extent of substrate hydrolysis.

One may use peptide sequences containing unnatural amino acids or modified bonds to construct the FRET-based substrate, or one may simply use the native peptide containing a protease cleavage site. Use of non-native sequences may be used to increase cleavage efficiency, to protect the peptide from degradation or to increase peptide solubility. For example, an ester linkage can replace an amide linkage in the peptide to increase cleavage efficiency.

One may use peptide sequences containing unnatural amino acids or modified bonds to construct the FRET-based substrate, or one may simply use the native peptide containing a protease cleavage site. Use of non-native sequences may be used to increase cleavage efficiency, to protect the peptide from degradation or to increase peptide solubility. For example, an ester linkage can replace an amide linkage in the peptide to increase cleavage efficiency.

Most fluorophores react with amino groups, meaning they can be conjugated to the α-amino group of Lysine or a free amine at the N-terminus of a peptide. The group on the fluorophore that reacts with an amine is typically an acylating group that can form a carboxamide, sulfonamide or thiourea. Nonlimiting examples of such reactive groups include: activated esters, acyl azides, acyl halides, acyl nitriles, anhydrides, isocyanates, isothiocyanates, sulfonate esters, sulfonyl halides.

Activated esters generally have the formula —COL, where L is a good leaving group (e.g., succinimidyloxy (—ONC₄H₄O₂), sulfosuccinimidyloxy(—ONC₄H₃O₂—SO₃H), -1-oxybenzotriazolyl (—OC₆H₄N₃). Or it can be an aryloxy group or aryloxy substituted one or more times by electron withdrawing substituents such as nitro, fluoro, chloro, cyano, or trifluoromethyl, or combinations thereof, used to form activated aryl esters. Or it can be a carboxylic acid activated by a carbodiimide to form an anhydride or mixed anhydride —OCOAlk or —OCN(Alk₁)NH(Alk₂), where Alk₁ and Alk₂, which may be the same or different, are C1-20 alkyl, C1-C20 perfluoroalkyl, or C1-C20 alkoxy, cycloheyxyl, 3-dimethylaminopropyl, or N-morpholinoethyl.

Where one is reacting a fluorophore with a lysine group, a reaction medium pH or 8.5 to 9.5 is usually optimal. In contrast, where one is reacting a fluorophore with an α-amino group at a peptide's N-terminus, reaction is typically conducted at near neutral pH.

Thiol reactive dyes can be used to conjugate donors and/or acceptors to Cys-containing peptides. The group on the fluorophore that reacts with the thiol is typically an alkylating reagent. Nonlimiting examples of alkylating reagents include iodoacetamides, maleimides, benzylic halides and bromomethylketones. Reaction of the alkylation reagents with a thiol usually proceeds well at or below room temperature at a pH between 6.5 to 8.0 to provide thioethers. This is an economical way to utilize the donors and/or acceptors since the peptides can be HPLC purified first before reacting with the dyes.

In certain cases, the donor and/or acceptor is directly conjugated to the peptide. For example, a carboxyl group on the donor and/or acceptor is activated—e.g., conversion to an activated ester—and reacted with an amine group on a peptide to form a carboxamide. In other cases, the donor and/or acceptor is conjugated to the peptide using a linking group. For example, a carboxyl group on the donor and/or acceptor is activated and reacted with an amine-containing spacer—e.g., NH₂-spacer-NHP, where “P” is a protecting group—to form a carboxamide of the general structure R—C(O)NH-spacer-NHP. The protecting group is removed to reveal an unprotected amine, which is then reacted with an activated carboxyl group on the target peptide.

For hydrophobic sequences, Lysines or Arginines may be added to increase solubility. These amino acids must be added at the appropriate positions without adversely affecting the protease recognition site.

Using the guidelines above, the inventors discovered a variety of optimal peptide-donor-acceptor combinations. Peptide substrates to which the donor and acceptor are conjugated include (“*” designating point of FRET moiety attachment):

| Activated Protein C: | |
|---|---|
| *-IEPRSFSQ-*; | (SEQ ID NO. 1) |
| *-LDRRGIQR-*. | (SEQ ID NO. 2) |
| ADAMTS-4 (Aggrecanase-1): | |
| *-AELQGRPISIA-*, | (SEQ ID NO. 3) |
| *-TEGEARGSVI-*. | (SEQ ID NO. 4) |
| ADAMTS-5 (Aggrecanase-2): | |
| *-TESESRGAIY-*. | (SEQ ID NO. 5) |
| ADAM-33: | |
| *-WEVAHQKLA-*, | (SEQ ID NO. 6) |
| *-WEVAHQK(*)LA. | |
| Alpha-Secretase: | |
| *-HQKLVFFA-*, | (SEQ ID NO. 7) |
| HQK(*)LVFFA-*. | |
| ADAM-17: | |
| -*SPLAQAVRSSSR-*; | (SEQ ID NO. 8) |
| *-LAQFVRSS-*. | (SEQ ID NO. 9) |
| ADAM-10: | |
| *-HGDQMAQKS-*, | (SEQ ID NO. 10) |
| *-HGDQMAQK(*)S. | |
| ADAM-12: | |
| *-LAQS-homoF-RS-*. | (SEQ ID NO. 11) |
| ADAM-8: | |
| *-HGDQMAQKS-*, | (SEQ ID NO. 12) |
| *-HGDQMAQK(*)S. | |
| ADAM-9: | |
| *-AVRSSSRG-*. | (SEQ ID NO. 13) |
| Angiotensin-converting enzyme (ACE2): | |
| *-YVADAPK-*. | (SEQ ID NO. 14) |
| Antiplasmin-cleaving enzyme (APCE): | |
| *-TSGPNQEQ-*. | (SEQ ID NO. 15) |
| 2A protease: | |
| *-DLTTYGFGH-*. | (SEQ ID NO. 16) |
| BACE 1: | |
| *-YEVHHQKLV-*, | (SEQ ID NO. 17) |
| *-YEVHHQK(*)LV; | |
| *-SEVNLDAEFR-*. | (SEQ ID NO. 18) |
| Bacterial Sortase: | |
| *-LPETG-*. | (SEQ ID NO. 19) |
| Calpain 1/2: | |
| *-PLLAERP-*; | (SEQ ID NO. 20) |
| *-TPLKSPPPSPR-*. | (SEQ ID NO. 21) |
| Caspase 1: | |
| *-YVADAPV-*. | (SEQ ID NO. 22) |
| Calpain 3: | |
| *-ISVDRPVK-*. | (SEQ ID NO. 23) |
| Carboxypeptidase E: | |
| *-GFARGG-*. | (SEQ ID NO. 24) |
| Carboxypeptidase M: | |
| *-GFLRGG-*. | (SEQ ID NO. 25) |
| Carboxypeptidase N: | |
| *-AGLQRGG-*. | (SEQ ID NO. 26) |
| Caspase 1: | |
| *-LESDYFGK-*. | (SEQ ID NO. 27) |
| Caspase 3: | |
| *-PFDLLDFN-*, | (SEQ ID NO. 28) |
| *-GDEVDGAK-*. | (SEQ ID NO. 29) |
| Caspase 6: | |
| *-NDTDANPR-*; | (SEQ ID NO. 30) |
| *-IQADSGPI-*. | (SEQ ID NO. 31) |
| Caspase 8: | |
| *-LQLDCVAV-*. | (SEQ ID NO. 32) |
| Cathepsin B: | |
| *-LLVYGG-*. | (SEQ ID NO. 33) |
| Cathepsin C: | |
| *-YDAKGD-*, | (SEQ ID NO. 34) |
| *-YDAK(*)GD. | |
| Cathepsin D: | |
| *-GKPILFFRL-*, | (SEQ ID NO. 35) |
| GK(*)PILFFRL-*; | |
| *-GKPIIFFRL-*, | (SEQ ID NO. 36) |
| GK(*)PIIFFRL-*; | |
| *-AAFFAA-*. | (SEQ ID NO. 37) |
| Cathepsin E: | |
| *-GKPILFFRL-*, | (SEQ ID NO. 38) |
| GK(*)PILFFRL-*. | |
| Cathepsin G: | |
| *-FVT-Gnf-SW-*; | (SEQ ID NO. 39) |
| *-DTDLYDYY-*. | (SEQ ID NO. 40) |
| Cathepsin K: | |
| *HPGGPQ-*. | (SEQ ID NO. 41) |
| Cathepsin L: | |
| *-EKARVLAEAA-*, | (SEQ ID NO. 42) |
| EK(*)ARVLAEAA-*. | |
| Cathepsin S: | |
| *-EKARVLAEAA-*, | (SEQ ID NO. 43) |
| EK(*)ARVLAEAA-*. | |
| Chymopapain: | |
| *-APVK-*. | (SEQ ID NO. 44) |
| Chymotrypsin: | |
| *-LAYGLRSK-*. | (SEQ ID NO. 45) |
| Complement component C1s: | |
| *-SLGRKIQIQ-*, | (SEQ ID NO. 46) |
| *-SLGRK(*)IQIQ, | |
| SLGRK(*)IQIQ-*. | |
| CMV Protease: | |
| *-RGVVNASSRL-*. | (SEQ ID NO. 47) |
| Endothelin-Converting Enzyme-1 (ECE-1): | |
| *-RPPGFSAF-*. | (SEQ ID NO. 48) |
| Factor Xa: | |
| *-GRGRGG-*; | (SEQ ID NO. 49) |
| *-FNPRTFGS-*. | (SEQ ID NO. 50) |
| Furin: | |
| *-RQKRFVLS-*, | (SEQ ID NO. 51) |
| RQK(*)RFVLS-*; | |
| *-RKRRSVNP-*, | (SEQ ID NO. 52) |
| RK(*)RRSVNP-*. | |
| Granzyme K: | |
| *-YGPKKKRK-*, | (SEQ ID NO. 53) |
| *-YGPKKK(*)RK, | |
| *-YGPKK(*)KRK, | |
| YGPK(*)KKRK-*. | |
| Hepatitis C virus NS3/4A protease: | |
| *-DDMEE-Abu-[COO]-AS-*. | (SEQ ID NO. 54) |
| Hepatitis C virus NS5A/5B protease: | |
| *-EDVVACSMSY-*, | (SEQ ID NO. 55) |
| *-EDVVAC(*)SMSY, | |
| *-EDVVAC(*)SMSY; | |
| *-VEDVVCCSM-*, | (SEQ ID NO. 56) |
| *-VEDVVCC(*)SM, | |
| *-VEDVVC(*)CSM. | |
| HIV protease: | |
| *-SQNYPIVQ-*. | (SEQ ID NO. 57) |
| HRV1: | |
| *-PPEELKFQ-*, | (SEQ ID NO. 58) |
| *-PPEELK(*)FQ; | |
| *-AIFQGPID-*. | (SEQ ID NO. 59) |
| h Kallikrein 1 (hK1): | |
| *-GFSPFRASRV-*. | (SEQ ID NO. 60) |
| h Kallikrein 2 (hK2): | |
| *-GKAFRR-*, | (SEQ ID NO. 61) |
| GK(*)AFRR-*. | |
| h Kallikrein 6 (hK6): | |
| *-AFRFSQ-*. | (SEQ ID NO. 62) |
| Interferon alpha A: | |
| *-GVGVTETPLM-*. | (SEQ ID NO. 63) |
| Lethal Factor Protease (LF): | |
| *-RRKKVYPYPMEGTIA-*, | (SEQ ID NO. 64) |
| RRKK(*)VYPYPMEGTIA-*, | |
| RRK(*)KVYPYPMEGTIA-*; | |
| *-PTLQLPLA-*. | (SEQ ID NO. 65) |
| Malaria Aspartyl Proteinase: | |
| *-ER-Nle-FLSFP-*; | (SEQ ID NO. 66) |
| *-ERMFLSFP-*; | (SEQ ID NO. 67) |
| *-ALERMFLSFP-*. | (SEQ ID NO. 68) |
| MMPs: | |
| *-KGP-Cha-Abu-Smc-HA-*. | (SEQ ID NO. 69) |
| Pepsin A: | |
| *-GSHLVEAL-*. | (SEQ ID NO. 70) |
| Plasmin: | |
| *-PKAKSHAP-*, | (SEQ ID NO. 71) |
| PK(*)AKSHAP-*, | |
| PKAK(*)SHAP-*; | |
| *-PQFRIKG-*, | (SEQ ID NO. 72) |
| *-PQFRIK(*)G. | |
| Plasmepsin II: | |
| *-ALERMFLSFP-*. | (SEQ ID NO. 73) |
| Proteinase 3 (PR3): | |
| *-VADVKDR-*, | (SEQ ID NO. 74) |
| *VADVK(*)DR; | |
| *-L2p-YDAKGD-*, | (SEQ ID NO. 75) |
| *-L2p-YDAK(*)GD. | |
| Proteinase A: | |
| *-APAKFFRL-*, | (SEQ ID NO. 76) |
| *-APAK(*)FFRL, | |
| APAK(*)FFRL-*. | |
| Proteinase K: | |
| *-STPVSSANMK-*. | (SEQ ID NO. 77) |
| Protein Tyrosine Phosphatase: | |
| *-EDAEPYAAK-*. | (SEQ ID NO. 78) |
| Renin: | |
| *-IHPFHLVIHT-*. | (SEQ ID NO. 79) |
| SARS: | |
| *-SVTLQSG-*; | (SEQ ID NO. 80) |
| *-TSAVLQSGFR-*; | (SEQ ID NO. 81) |
| *-FRLKGGAPIKGV-*, | (SEQ ID NO. 82) |
| FRLK(*)GGAPIKGV-*, | |
| FRLK(*)GGAPIK(*)GV, | |
| *-FRLKGGAPIK(*)GV. | |
| TACE: | |
| *-SPLAQAVRSSSR-*. | (SEQ ID NO. 83) |
| Thrombin: | |
| *-DYVPRGSKD-*, | (SEQ ID NO. 84) |
| *-DYVPRGSK(*)D; | |
| *-IEPRSFSQ-*; | (SEQ ID NO. 85) |
| *-GVPRSFRG-*. | (SEQ ID NO. 86) |
| Tobacco Etch Virus Protease (TEV): | |
| *-ENLYFQSGTK-*; | (SEQ ID NO. 87) |
| *-LYFQSGTV-*. | (SEQ ID NO. 88) |
| Trypsin: | |
| *-GRGHARLV-*; | (SEQ ID NO. 89) |
| *-GHARLVHG-*; | (SEQ ID NO. 90) |
| *-GPAKLAIG-*, | (SEQ ID NO. 91) |
| GPAK(*)LAIG-*, | |
| *-GPAK(*)LAIG. | |
| West Nile Virus Protease: | |
| *-FASGKRSQIGL-*, | (SEQ ID NO. 92) |
| *-FASGK(*)RSQIGL, | |
| FASGK(*)RSQIGL-*. | |

FRET substrates—i.e., peptide/donor/acceptor combinations—according to the present invention are shown below (“A1” through “A5” and “B1” through “B10” designating moieties of a FRET pair, with the moiety attached through a reaction with the carboxylic acid functionality):

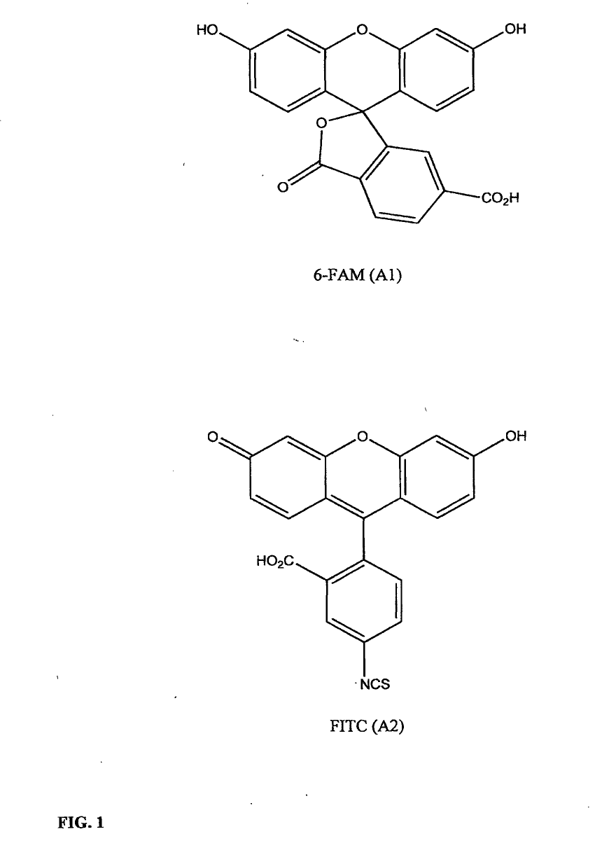

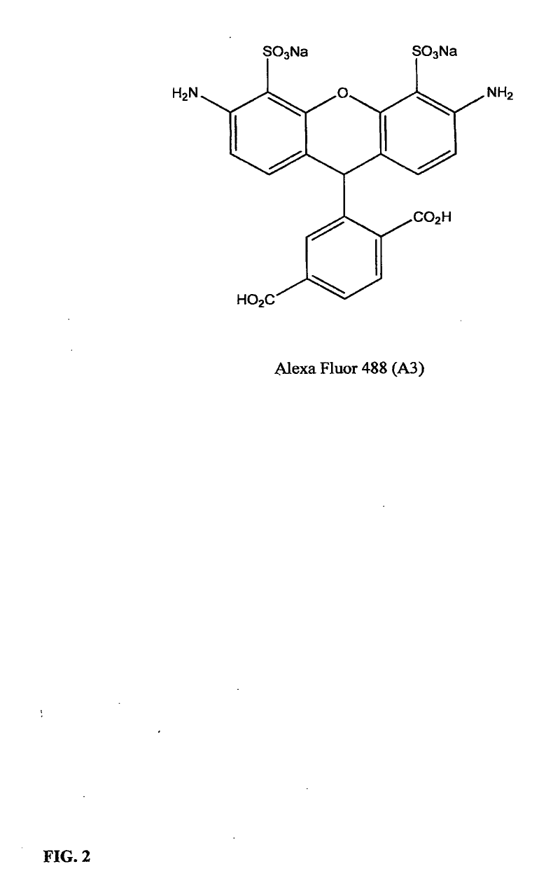

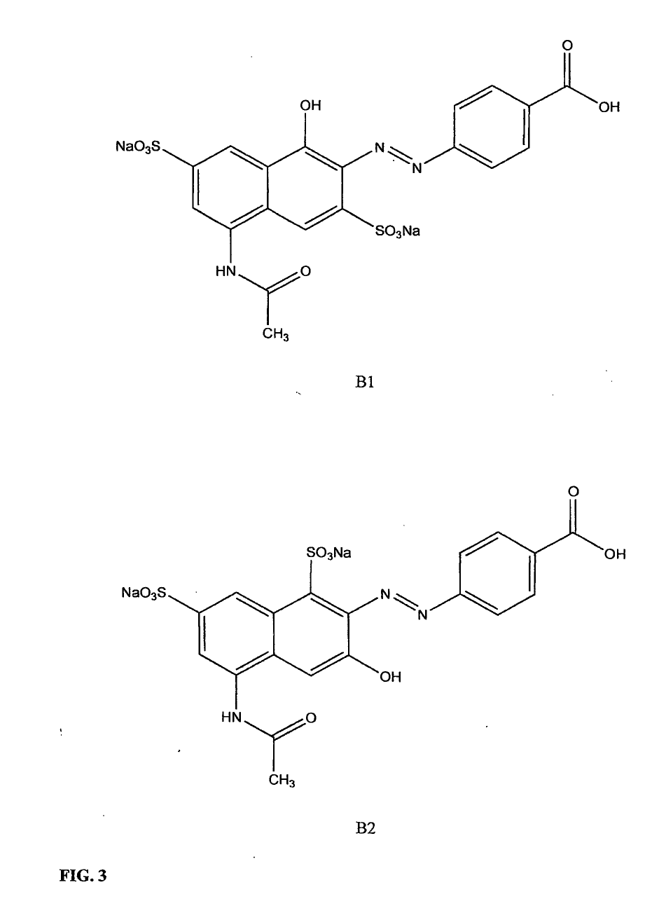

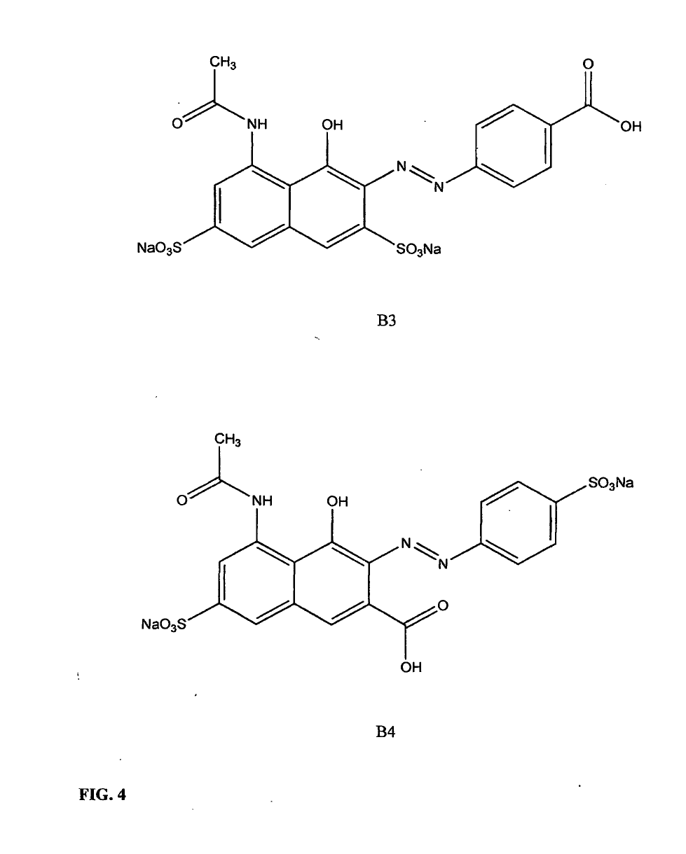

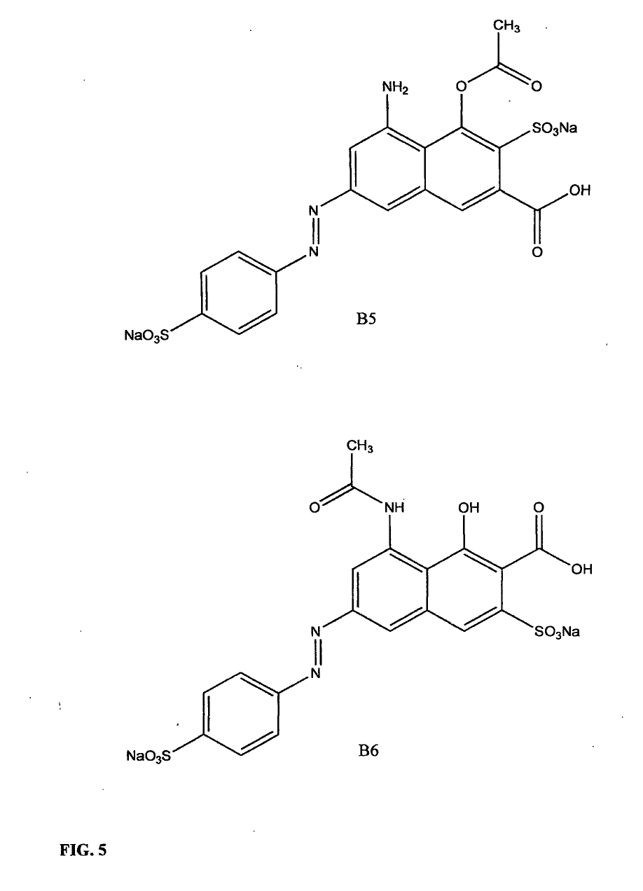

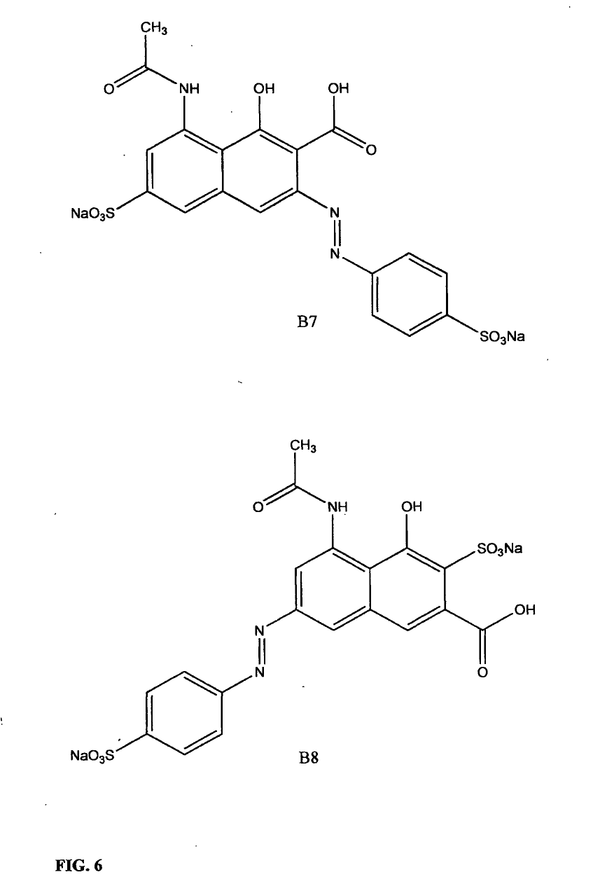

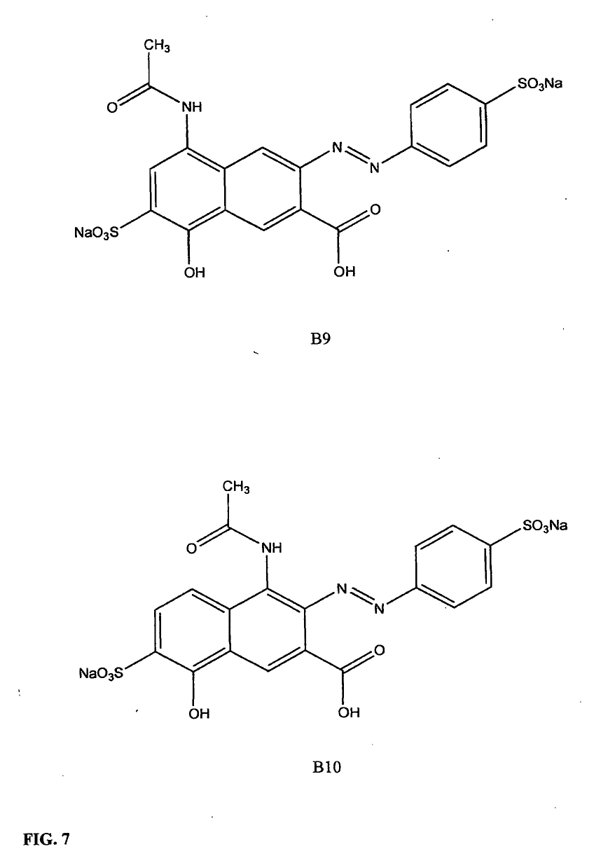

FIG. 1 shows the structure of 6-FAM designated as “A1” and FITC designated as “A2”. FIG. 2 shows the structure of Alexa Fluor 488 designated as “A3”. “A4” refers to Dylight Fluor 488, which is a green fluorescent dye of mass 1011 g/mol and exhibits peak absorption at 493 nm and peak emission at 518 nm. “A5” refers to HiLyte Fluor 488, which is a fluorescent dye of mass 530.45 g/mol and exhibits peak absorption at 503 nm and peak emission at 428 nm. FIG. 3 shows the structure of two compounds that are acceptor moieties in a FRET pair according to the present invention. The compounds are respectively designated as “B1” and “B2”. FIG. 4 shows the structure of two compounds that are acceptor moieties in a FRET pair according to the present invention. The compounds are respectively designated as “B3” and “B4”. FIG. 5 shows the structure of two compounds that are acceptor moieties in a FRET pair according to the present invention. The compounds are respectively designated as “B5” and “B6”. FIG. 6 shows the structure of two compounds that are acceptor moieties in a FRET pair according to the present invention. The compounds are respectively designated as “B7” and “B8”. FIG. 7 shows the structure of two compounds that are acceptor moieties in a FRET pair according to the present invention. The compounds are respectively designated as “B9” and “B10”.

A1-IEPRSFSQ-B1; A1-IEPRSFSQ-B2; A1-IEPRSFSQ-B3; A1-IEPRSFSQ-B4; A1-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10; A2-IEPRSFSQ-B1; A2-IEPRSFSQ-B2; A2-IEPRSFSQ-B3; A2-IEPRSFSQ-B4; A2-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10; A3-IEPRSFSQ-B1; A3-IEPRSFSQ-B2; A3-IEPRSFSQ-B2; A3-IEPRSFSQ-B3; A3-IEPRSFSQ-B4; A3-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10; A4-IEPRSFSQ-B1; A4-IEPRSFSQ-B2; A4-IEPRSFSQ-B2; A4-IEPRSFSQ-B3; A4-IEPRSFSQ-B4; A4-IEPRSFSQ-B5; A5-IEPRSFSQ-B1; A5-IEPRSFSQ-B2; A5-IEPRSFSQ-B2; A5-IEPRSFSQ-B3; A5-IEPRSFSQ-B4; A5-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10.

A1-LDRRGIQR-B1; A1-LDRRGIQR-B2; A1-LDRRGIQR-B3; A1-LDRRGIQR-B4; A1-LDRRGIQR-B5; A1-LDRRGIQR-B6; A1-LDRRGIQR-B7; A1-LDRRGIQR-B8; A1-LDRRGIQR-B9; A1-LDRRGIQR-B10; A2-LDRRGIQR-B1; A2-LDRRGIQR-B2; A2-LDRRGIQR-B3; A2-LDRRGIQR-B4; A2-LDRRGIQR-B5; A1-LDRRGIQR-B6; A1-LDRRGIQR-B7; A1-LDRRGIQR-B8; A1-LDRRGIQR-B9; A1-LDRRGIQR-B10; A3-LDRRGIQR-B1; A3-LDRRGIQR-B2; A3-LDRRGIQR-B2; A3-LDRRGIQR-B3; A3-LDRRGIQR-B4; A3-LDRRGIQR-B5; A1-LDRRGIQR-B6; A1-LDRRGIQR-B7; A1-LDRRGIQR-B8; A1-LDRRGIQR-B9; A1-LDRRGIQR-B10; A4-LDRRGIQR-B1; A4-LDRRGIQR-B2; A4-LDRRGIQR-B2; A4-LDRRGIQR-B3; A4-LDRRGIQR-B4; A4-LDRRGIQR-B5; A5-IEPRSFSQ-B1; A5-IEPRSFSQ-B2; A5-IEPRSFSQ-B2; A5-IEPRSFSQ-B3; A5-IEPRSFSQ-B4; A5-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10.

A1-AELQGRPISIA-B1; A1-AELQGRPISIA-B2; A1-AELQGRPISIA-B3; A1-AELQGRPISIA-B4; A1-AELQGRPISIA-B5; A1-AELQGRPISIA-B6; A1-AELQGRPISIA-B7; A1-AELQGRPISIA-B8; A1-AELQGRPISIA-B9; A1-AELQGRPISIA-B10; A2-AELQGRPISIA-B1; A2-AELQGRPISIA-B2; A2-AELQGRPISIA-B3; A2-AELQGRPISIA-B4; A2-AELQGRPISIA-B5; A1-AELQGRPISIA-B6; A1-AELQGRPISIA-B7; A1-AELQGRPISIA-B8; A1-AELQGRPISIA-B9; A1-AELQGRPISIA-B10; A3-AELQGRPISIA-B1; A3-AELQGRPISIA-B2; A3-AELQGRPISIA-B2; A3-AELQGRPISIA-B3; A3-AELQGRPISIA-B4; A3-AELQGRPISIA-B5; A1-AELQGRPISIA-B6; A1-AELQGRPISIA-B7; A1-AELQGRPISIA-B8; A1-AELQGRPISIA-B9; A1-AELQGRPISIA-B10; A4-AELQGRPISIA-B1; A4-AELQGRPISIA-B2; A4-AELQGRPISIA-B2; A4-AELQGRPISIA-B3; A4-AELQGRPISIA-B4; A4-AELQGRPISIA-B5; A5-AELQGRPISIA-B1; A5-AELQGRPISIA-B2; A5-AELQGRPISIA-B2; A5-AELQGRPISIA-B3; A5-AELQGRPISIA-B4; A5-AELQGRPISIA-B5; A1-AELQGRPISIA-B6; A1-AELQGRPISIA-B7; A1-AELQGRPISIA-B8; A1-AELQGRPISIA-B9; A1-AELQGRPISIA-B10.

A1-TEGEARGSVI-B1; A1-TEGEARGSVI-B2; A1-TEGEARGSVI-B3; A1-TEGEARGSVI-B4; A1-TEGEARGSVI-B5; A1-TEGEARGSVI-B6; A1-TEGEARGSVI-B7; A1-TEGEARGSVI-B8; A1-TEGEARGSVI-B9; A1-TEGEARGSVI-B10; A2-TEGEARGSVI-B1; A2-TEGEARGSVI-B2; A2-TEGEARGSVI-B3; A2-TEGEARGSVI-B4; A2-TEGEARGSVI-B5; A1-TEGEARGSVI-B6; A1-TEGEARGSVI-B7; A1-TEGEARGSVI-B8; A1-TEGEARGSVI-B9; A1-TEGEARGSVI-B10; A3-TEGEARGSVI-B1; A3-TEGEARGSVI-B2; A3-TEGEARGSVI-B2; A3-TEGEARGSVI-B3; A3-TEGEARGSVI-B4; A3-TEGEARGSVI-B5; A1-TEGEARGSVI-B6; A1-TEGEARGSVI-B7; A1-TEGEARGSVI-B8; A1-TEGEARGSVI-B9; A1-TEGEARGSVI-B10; A4-TEGEARGSVI-B1; A4-TEGEARGSVI-B2; A4-TEGEARGSVI-B2; A4-TEGEARGSVI-B3; A4-TEGEARGSVI-B4; A4-TEGEARGSVI-B5; A5-TEGEARGSVI-B1; A5-TEGEARGSVI-B2; A5-TEGEARGSVI-B2; A5-TEGEARGSVI-B3; A5-TEGEARGSVI-B4; A5-TEGEARGSVI-B5; A1-TEGEARGSVI-B6; A1-TEGEARGSVI-B7; A1-TEGEARGSVI-B8; A1-TEGEARGSVI-B9; A1-TEGEARGSVI-B10.

A1-TESESRGAIY-B1; A1-TESESRGAIY-B2; A1-TESESRGAIY-B3; A1-TESESRGAIY-B4; A1-TESESRGAIY-B5; A1-TESESRGAIY-B6; A1-TESESRGAIY-B7; A1-TESESRGAIY-B8; A1-TESESRGAIY-B9; A1-TESESRGAIY-B10; A2-TESESRGAIY-B1; A2-TESESRGAIY-B2; A2-TESESRGAIY-B3; A2-TESESRGAIY-B4; A2-TESESRGAIY-B5; A1-TESESRGAIY-B6; A1-TESESRGAIY-B7; A1-TESESRGAIY-B8; A1-TESESRGAIY-B9; A1-TESESRGAIY-B10; A3-TESESRGAIY-B1; A3-TESESRGAIY-B2; A3-TESESRGAIY-B2; A3-TESESRGAIY-B3; A3-TESESRGAIY-B4; A3-TESESRGAIY-B5; A1-TESESRGAIY-B6; A1-TESESRGAIY-B7; A1-TESESRGAIY-B8; A1-TESESRGAIY-B9; A1-TESESRGAIY-B10; A4-TESESRGAIY-B1; A4-TESESRGAIY-B2; A4-TESESRGAIY-B2; A4-TESESRGAIY-B3; A4-TESESRGAIY-B4; A4-TESESRGAIY-B5; A5-TESESRGAIY-B1; A5-TESESRGAIY-B2; A5-TESESRGAIY-B2; A5-TESESRGAIY-B3; A5-TESESRGAIY-B4; A5-TESESRGAIY-B5; A1-TESESRGAIY-B6; A1-TESESRGAIY-B7; A1-TESESRGAIY-B8; A1-TESESRGAIY-B9; A1-TESESRGAIY-B10.

A1-SPLAQAVRSSSR-B1; A1-SPLAQAVRSSSR-B2; A1-SPLAQAVRSSSR-B3; A1-SPLAQAVRSSSR-B4; A1-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A2-SPLAQAVRSSSR-B1; A2-SPLAQAVRSSSR-B2; A2-SPLAQAVRSSSR-B3; A2-SPLAQAVRSSSR-B4; A2-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A3-SPLAQAVRSSSR-B1; A3-SPLAQAVRSSSR-B2; A3-SPLAQAVRSSSR-B2; A3-SPLAQAVRSSSR-B3; A3-SPLAQAVRSSSR-B4; A3-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A4-SPLAQAVRSSSR-B1; A4-SPLAQAVRSSSR-B2; A4-SPLAQAVRSSSR-B2; A4-SPLAQAVRSSSR-B3; A4-SPLAQAVRSSSR-B4; A4-SPLAQAVRSSSR-B5; A5-SPLAQAVRSSSR-B1; A5-SPLAQAVRSSSR-B2; A5-SPLAQAVRSSSR-B2; A5-SPLAQAVRSSSR-B3; A5-SPLAQAVRSSSR-B4; A5-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10.

A1-WEVAHQKLA-B1; A1-WEVAHQKLA-B2; A1-WEVAHQKLA-B3; A1-WEVAHQKLA-B4; A1-WEVAHQKLA-B5; A1-WEVAHQKLA-B6; A1-WEVAHQKLA-B7; A1-WEVAHQKLA-B8; A1-WEVAHQKLA-B9; A1-WEVAHQKLA-B10; A2-WEVAHQKLA-B1; A2-WEVAHQKLA-B2; A2-WEVAHQKLA-B3; A2-WEVAHQKLA-B4; A2-WEVAHQKLA-B5; A1-WEVAHQKLA-B6; A1-WEVAHQKLA-B7; A1-WEVAHQKLA-B8; A1-WEVAHQKLA-B9; A1-WEVAHQKLA-B10; A3-WEVAHQKLA-B1; A3-WEVAHQKLA-B2; A3-WEVAHQKLA-B2; A3-WEVAHQKLA-B3; A3-WEVAHQKLA-B4; A3-WEVAHQKLA-B5; A1-WEVAHQKLA-B6; A1-WEVAHQKLA-B7; A1-WEVAHQKLA-B8; A1-WEVAHQKLA-B9; A1-WEVAHQKLA-B10; A4-WEVAHQKLA-B1; A4-WEVAHQKLA-B2; A4-WEVAHQKLA-B2; A4-WEVAHQKLA-B3; A4-WEVAHQKLA-B4; A4-WEVAHQKLA-B5; A5-WEVAHQKLA-B1; A5-WEVAHQKLA-B2; A5-WEVAHQKLA-B2; A5-WEVAHQKLA-B3; A5-WEVAHQKLA-B4; A5-WEVAHQKLA-B5; A1-WEVAHQKLA-B6; A1-WEVAHQKLA-B7; A1-WEVAHQKLA-B8; A1-WEVAHQKLA-B9; A1-WEVAHQKLA-B10.

A1-HQKLVFFA-B1; A1-HQKLVFFA-B2; A1-HQKLVFFA-B3; A1-HQKLVFFA-B4; A1-HQKLVFFA-B5; A1-HQKLVFFA-B6; A1-HQKLVFFA-B7; A1-HQKLVFFA-B8; A1-HQKLVFFA-B9; A1-HQKLVFFA-B10; A2-HQKLVFFA-B1; A2-HQKLVFFA-B2; A2-HQKLVFFA-B3; A2-HQKLVFFA-B4; A2-HQKLVFFA-B5; A1-HQKLVFFA-B6; A1-HQKLVFFA-B7; A1-HQKLVFFA-B8; A1-HQKLVFFA-B9; A1-HQKLVFFA-B10; A3-HQKLVFFA-B1; A3-HQKLVFFA-B2; A3-HQKLVFFA-B2; A3-HQKLVFFA-B3; A3-HQKLVFFA-B4; A3-HQKLVFFA-B5; A1-HQKLVFFA-B6; A1-HQKLVFFA-B7; A1-HQKLVFFA-B8; A1-HQKLVFFA-B9; A1-HQKLVFFA-B10; A4-HQKLVFFA-B1; A4-HQKLVFFA-B2; A4-HQKLVFFA-B2; A4-HQKLVFFA-B3; A4-HQKLVFFA-B4; A4-HQKLVFFA-B5; A5-HQKLVFFA-B1; A5-HQKLVFFA-B2; A5-HQKLVFFA-B2; A5-HQKLVFFA-B3; A5-HQKLVFFA-B4; A5-HQKLVFFA-B5; A1-HQKLVFFA-B6; A1-HQKLVFFA-B7; A1-HQKLVFFA-B8; A1-HQKLVFFA-B9; A1-HQKLVFFA-B10.

A1-SPLAQAVRSSSR-B1; A1-SPLAQAVRSSSR-B2; A1-SPLAQAVRSSSR-B3; A1-SPLAQAVRSSSR-B4; A1-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A2-SPLAQAVRSSSR-B1; A2-SPLAQAVRSSSR-B2; A2-SPLAQAVRSSSR-B3; A2-SPLAQAVRSSSR-B4; A2-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A3-SPLAQAVRSSSR-B1; A3-SPLAQAVRSSSR-B2; A3-SPLAQAVRSSSR-B2; A3-SPLAQAVRSSSR-B3; A3-SPLAQAVRSSSR-B4; A3-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A4-SPLAQAVRSSSR-B1; A4-SPLAQAVRSSSR-B2; A4-SPLAQAVRSSSR-B2; A4-SPLAQAVRSSSR-B3; A4-SPLAQAVRSSSR-B4; A4-SPLAQAVRSSSR-B5; A5-SPLAQAVRSSSR-B1; A5-SPLAQAVRSSSR-B2; A5-SPLAQAVRSSSR-B2; A5-SPLAQAVRSSSR-B3; A5-SPLAQAVRSSSR-B4; A5-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10.

A1-LAQFVRSS-B1; A1-LAQFVRSS-B2; A1-LAQFVRSS-B3; A1-LAQFVRSS-B4; A1-LAQFVRSS-B5; A1-LAQFVRSS-B6; A1-LAQFVRSS-B7; A1-LAQFVRSS-B8; A1-LAQFVRSS-B9; A1-LAQFVRSS-B10; A2-LAQFVRSS-B1; A2-LAQFVRSS-B2; A2-LAQFVRSS-B3; A2-LAQFVRSS-B4; A2-LAQFVRSS-B5; A1-LAQFVRSS-B6; A1-LAQFVRSS-B7; A1-LAQFVRSS-B8; A1-LAQFVRSS-B9; A1-LAQFVRSS-B10; A3-LAQFVRSS-B1; A3-LAQFVRSS-B2; A3-LAQFVRSS-B2; A3-LAQFVRSS-B3; A3-LAQFVRSS-B4; A3-LAQFVRSS-B5; A1-LAQFVRSS-B6; A1-LAQFVRSS-B7; A1-LAQFVRSS-B8; A1-LAQFVRSS-B9; A1-LAQFVRSS-B10; A4-LAQFVRSS-B1; A4-LAQFVRSS-B2; A4-LAQFVRSS-B2; A4-LAQFVRSS-B3; A4-LAQFVRSS-B4; A4-LAQFVRSS-B5; A5-LAQFVRSS-B1; A5-LAQFVRSS-B2; A5-LAQFVRSS-B2; A5-LAQFVRSS-B3; A5-LAQFVRSS-B4; A5-LAQFVRSS-B5; A1-LAQFVRSS-B6; A1-LAQFVRSS-B7; A1-LAQFVRSS-B8; A1-LAQFVRSS-B9; A1-LAQFVRSS-B10.

A1-HGDQMAQKS-B1; A1-HGDQMAQKS-B2; A1-HGDQMAQKS-B3; A1-HGDQMAQKS-B4; A1-HGDQMAQKS-B5; A1-HGDQMAQKS-B6; A1-HGDQMAQKS-B7; A1-HGDQMAQKS-B8; A1-HGDQMAQKS-B9; A1-HGDQMAQKS-B10; A2-HGDQMAQKS-B1; A2-HGDQMAQKS-B2; A2-HGDQMAQKS-B3; A2-HGDQMAQKS-B4; A2-HGDQMAQKS-B5; A1-HGDQMAQKS-B6; A1-HGDQMAQKS-B7; A1-HGDQMAQKS-B8; A1-HGDQMAQKS-B9; A1-HGDQMAQKS-B10; A3-HGDQMAQKS-B1; A3-HGDQMAQKS-B2; A3-HGDQMAQKS-B2; A3-HGDQMAQKS-B3; A3-HGDQMAQKS-B4; A3-HGDQMAQKS-B5; A1-HGDQMAQKS-B6; A1-HGDQMAQKS-B7; A1-HGDQMAQKS-B8; A1-HGDQMAQKS-B9; A1-HGDQMAQKS-B10; A4-HGDQMAQKS-B1; A4-HGDQMAQKS-B2; A4-HGDQMAQKS-B2; A4-HGDQMAQKS-B3; A4-HGDQMAQKS-B4; A4-HGDQMAQKS-B5; A5-HGDQMAQKS-B1; A5-HGDQMAQKS-B2; A5-HGDQMAQKS-B2; A5-HGDQMAQKS-B3; A5-HGDQMAQKS-B4; A5-HGDQMAQKS-B5; A1-HGDQMAQKS-B6; A1-HGDQMAQKS-B7; A1-HGDQMAQKS-B8; A1-HGDQMAQKS-B9; A1-HGDQMAQKS-B10.

A1-LAQS-homoF-RS-B1; A1-LAQS-homoF-RS-B2; A1-LAQS-homoF-RS-B3; A1-LAQS-homoF-RS-B4; A1-LAQS-homoF-RS-B5; A1-LAQS-homoF-RS-B6; A1-LAQS-homoF-RS-B7; A1-LAQS-homoF-RS-B8; A1-LAQS-homoF-RS-B9; A1-LAQS-homoF-RS-B10; A2-LAQS-homoF-RS-B1; A2-LAQS-homoF-RS-B2; A2-LAQS-homoF-RS-B3; A2-LAQS-homoF-RS-B4; A2-LAQS-homoF-RS-B5; A1-LAQS-homoF-RS-B6; A1-LAQS-homoF-RS-B7; A1-LAQS-homoF-RS-B8; A1-LAQS-homoF-RS-B9; A1-LAQS-homoF-RS-B10; A3-LAQS-homoF-RS-B1; A3-LAQS-homoF-RS-B2; A3-LAQS-homoF-RS-B2; A3-LAQS-homoF-RS-B3; A3-LAQS-homoF-RS-B4; A3-LAQS-homoF-RS-B5; A1-LAQS-homoF-RS-B6; A1-LAQS-homoF-RS-B7; A1-LAQS-homoF-RS-B8; A1-LAQS-homoF-RS-B9; A1-LAQS-homoF-RS-B10; A4-LAQS-homoF-RS-B1; A4-LAQS-homoF-RS-B2; A4-LAQS-homoF-RS-B2; A4-LAQS-homoF-RS-B3; A4-LAQS-homoF-RS-B4; A4-LAQS-homoF-RS-B5; A5-LAQS-homoF-RS-B1; A5-LAQS-homoF-RS-B2; A5-LAQS-homoF-RS-B2; A5-LAQS-homoF-RS-B3; A5-LAQS-homoF-RS-B4; A5-LAQS-homoF-RS-B5; A1-LAQS-homoF-RS-B6; A1-LAQS-homoF-RS-B7; A1-LAQS-homoF-RS-B8; A1-LAQS-homoF-RS-B9; A1-LAQS-homoF-RS-B10.

A1-HGDQMAQKS-B1; A1-HGDQMAQKS-B2; A1-HGDQMAQKS-B3; A1-HGDQMAQKS-B4; A1-HGDQMAQKS-B5; A1-HGDQMAQKS-B6; A1-HGDQMAQKS-B7; A1-HGDQMAQKS-B8; A1-HGDQMAQKS-B9; A1-HGDQMAQKS-B10; A2-HGDQMAQKS-B1; A2-HGDQMAQKS-B2; A2-HGDQMAQKS-B3; A2-HGDQMAQKS-B4; A2-HGDQMAQKS-B5; A1-HGDQMAQKS-B6; A1-HGDQMAQKS-B7; A1-HGDQMAQKS-B8; A1-HGDQMAQKS-B9; A1-HGDQMAQKS-B10; A3-HGDQMAQKS-B1; A3-HGDQMAQKS-B2; A3-HGDQMAQKS-B2; A3-HGDQMAQKS-B3; A3-HGDQMAQKS-B4; A3-HGDQMAQKS-B5; A1-HGDQMAQKS-B6; A1-HGDQMAQKS-B7; A1-HGDQMAQKS-B8; A1-HGDQMAQKS-B9; A1-HGDQMAQKS-B10; A4-HGDQMAQKS-B1; A4-HGDQMAQKS-B2; A4-HGDQMAQKS-B2; A4-HGDQMAQKS-B3; A4-HGDQMAQKS-B4; A4-HGDQMAQKS-B5; A5-HGDQMAQKS-B1; A5-HGDQMAQKS-B2; A5-HGDQMAQKS-B2; A5-HGDQMAQKS-B3; A5-HGDQMAQKS-B4; A5-HGDQMAQKS-B5; A1-HGDQMAQKS-B6; A1-HGDQMAQKS-B7; A1-HGDQMAQKS-B8; A1-HGDQMAQKS-B9; A1-HGDQMAQKS-B10.

A1-AVRSSSRG-B1; A1-AVRSSSRG-B2; A1-AVRSSSRG-B3; A1-AVRSSSRG-B4; A1-AVRSSSRG-B5; A1-AVRSSSRG-B6; A1-AVRSSSRG-B7; A1-AVRSSSRG-B8; A1-AVRSSSRG-B9; A1-AVRSSSRG-B10; A2-AVRSSSRG-B1; A2-AVRSSSRG-B2; A2-AVRSSSRG-B3; A2-AVRSSSRG-B4; A2-AVRSSSRG-B5; A1-AVRSSSRG-B6; A1-AVRSSSRG-B7; A1-AVRSSSRG-B8; A1-AVRSSSRG-B9; A1-AVRSSSRG-B10; A3-AVRSSSRG-B1; A3-AVRSSSRG-B2; A3-AVRSSSRG-B2; A3-AVRSSSRG-B3; A3-AVRSSSRG-B4; A3-AVRSSSRG-B5; A1-AVRSSSRG-B6; A1-AVRSSSRG-B7; A1-AVRSSSRG-B8; A1-AVRSSSRG-B9; A1-AVRSSSRG-B10; A4-AVRSSSRG-B1; A4-AVRSSSRG-B2; A4-AVRSSSRG-B2; A4-AVRSSSRG-B3; A4-AVRSSSRG-B4; A4-AVRSSSRG-B5; A5-AVRSSSRG-B1; A5-AVRSSSRG-B2; A5-AVRSSSRG-B2; A5-AVRSSSRG-B3; A5-AVRSSSRG-B4; A5-AVRSSSRG-B5; A1-AVRSSSRG-B6; A1-AVRSSSRG-B7; A1-AVRSSSRG-B8; A1-AVRSSSRG-B9; A1-AVRSSSRG-B10.

A1-YVADAPK-B1; A1-YVADAPK-B2; A1-YVADAPK-B3; A1-YVADAPK-B4; A1-YVADAPK-B5; A1-YVADAPK-B6; A1-YVADAPK-B7; A1-YVADAPK-B8; A1-YVADAPK-B9; A1-YVADAPK-B10; A2-YVADAPK-B1; A2-YVADAPK-B2; A2-YVADAPK-B3; A2-YVADAPK-B4; A2-YVADAPK-B5; A1-YVADAPK-B6; A1-YVADAPK-B7; A1-YVADAPK-B8; A1-YVADAPK-B9; A1-YVADAPK-B10; A3-YVADAPK-B1; A3-YVADAPK-B2; A3-YVADAPK-B2; A3-YVADAPK-B3; A3-YVADAPK-B4; A3-YVADAPK-B5; A1-YVADAPK-B6; A1-YVADAPK-B7; A1-YVADAPK-B8; A1-YVADAPK-B9; A1-YVADAPK-B10; A4-YVADAPK-B1; A4-YVADAPK-B2; A4-YVADAPK-B2; A4-YVADAPK-B3; A4-YVADAPK-B4; A4-YVADAPK-B5; A5-YVADAPK-B1; A5-YVADAPK-B2; A5-YVADAPK-B2; A5-YVADAPK-B3; A5-YVADAPK-B4; A5-YVADAPK-B5; A1-YVADAPK-B6; A1-YVADAPK-B7; A1-YVADAPK-B8; A1-YVADAPK-B9; A1-YVADAPK-B10.

A1-TSGPNQEQ-B1; A1-TSGPNQEQ-B2; A1-TSGPNQEQ-B3; A1-TSGPNQEQ-B4; A1-TSGPNQEQ-B5; A1-TSGPNQEQ-B6; A1-TSGPNQEQ-B7; A1-TSGPNQEQ-B8; A1-TSGPNQEQ-B9; A1-TSGPNQEQ-B10; A2-TSGPNQEQ-B1; A2-TSGPNQEQ-B2; A2-TSGPNQEQ-B3; A2-TSGPNQEQ-B4; A2-TSGPNQEQ-B5; A1-TSGPNQEQ-B6; A1-TSGPNQEQ-B7; A1-TSGPNQEQ-B8; A1-TSGPNQEQ-B9; A1-TSGPNQEQ-B10; A3-TSGPNQEQ-B1; A3-TSGPNQEQ-B2; A3-TSGPNQEQ-B2; A3-TSGPNQEQ-B3; A3-TSGPNQEQ-B4; A3-TSGPNQEQ-B5; A1-TSGPNQEQ-B6; A1-TSGPNQEQ-B7; A1-TSGPNQEQ-B8; A1-TSGPNQEQ-B9; A1-TSGPNQEQ-B10; A4-TSGPNQEQ-B1; A4-TSGPNQEQ-B2; A4-TSGPNQEQ-B2; A4-TSGPNQEQ-B3; A4-TSGPNQEQ-B4; A4-TSGPNQEQ-B5; A5-TSGPNQEQ-B1; A5-TSGPNQEQ-B2; A5-TSGPNQEQ-B2; A5-TSGPNQEQ-B3; A5-TSGPNQEQ-B4; A5-TSGPNQEQ-B5; A1-TSGPNQEQ-B6; A1-TSGPNQEQ-B7; A1-TSGPNQEQ-B8; A1-TSGPNQEQ-B9; A1-TSGPNQEQ-B 10.

A1-DLTTYGFGH-B1; A1-DLTTYGFGH-B2; A1-DLTTYGFGH-B3; A1-DLTTYGFGH-B4; A1-DLTTYGFGH-B5; A1-DLTTYGFGH-B6; A1-DLTTYGFGH-B7; A1-DLTTYGFGH-B8; A1-DLTTYGFGH-B9; A1-DLTTYGFGH-B10; A2-DLTTYGFGH-B1; A2-DLTTYGFGH-B2; A2-DLTTYGFGH-B3; A2-DLTTYGFGH-B4; A2-DLTTYGFGH-B5; A1-DLTTYGFGH-B6; A1-DLTTYGFGH-B7; A1-DLTTYGFGH-B8; A1-DLTTYGFGH-B9; A1-DLTTYGFGH-B10; A3-DLTTYGFGH-B1; A3-DLTTYGFGH-B2; A3-DLTTYGFGH-B2; A3-DLTTYGFGH-B3; A3-DLTTYGFGH-B4; A3-DLTTYGFGH-B5; A1-DLTTYGFGH-B6; A1-DLTTYGFGH-B7; A1-DLTTYGFGH-B8; A1-DLTTYGFGH-B9; A1-DLTTYGFGH-B10; A4-DLTTYGFGH-B1; A4-DLTTYGFGH-B2; A4-DLTTYGFGH-B2; A4-DLTTYGFGH-B3; A4-DLTTYGFGH-B4; A4-DLTTYGFGH-B5; A5-DLTTYGFGH-B1; A5-DLTTYGFGH-B2; A5-DLTTYGFGH-B2; A5-DLTTYGFGH-B3; A5-DLTTYGFGH-B4; A5-DLTTYGFGH-B5; A1-DLTTYGFGH-B6; A1-DLTTYGFGH-B7; A1-DLTTYGFGH-B8; A1-DLTTYGFGH-B9; A1-DLTTYGFGH-B10.

A1-YEVHHQKLV-B1; A1-YEVHHQKLV-B2; A1-YEVHHQKLV-B3; A1-YEVHHQKLV-B4; A1-YEVHHQKLV-B5; A1-YEVHHQKLV-B6; A1-YEVHHQKLV-B7; A1-YEVHHQKLV-B8; A1-YEVHHQKLV-B9; A1-YEVHHQKLV-B10; A2-YEVHHQKLV-B1; A2-YEVHHQKLV-B2; A2-YEVHHQKLV-B3; A2-YEVHHQKLV-B4; A2-YEVHHQKLV-B5; A1-YEVHHQKLV-B6; A1-YEVHHQKLV-B7; A1-YEVHHQKLV-B8; A1-YEVHHQKLV-B9; A1-YEVHHQKLV-B10; A3-YEVHHQKLV-B1; A3-YEVHHQKLV-B2; A3-YEVHHQKLV-B2; A3-YEVHHQKLV-B3; A3-YEVHHQKLV-B4; A3-YEVHHQKLV-B5; A1-YEVHHQKLV-B6; A1-YEVHHQKLV-B7; A1-YEVHHQKLV-B8; A1-YEVHHQKLV-B9; A1-YEVHHQKLV-B10; A4-YEVHHQKLV-B1; A4-YEVHHQKLV-B2; A4-YEVHHQKLV-B2; A4-YEVHHQKLV-B3; A4-YEVHHQKLV-B4; A4-YEVHHQKLV-B5; A5-YEVHHQKLV-B1; A5-YEVHHQKLV-B2; A5-YEVHHQKLV-B2; A5-YEVHHQKLV-B3; A5-YEVHHQKLV-B4; A5-YEVHHQKLV-B5; A1-YEVHHQKLV-B6; A1-YEVHHQKLV-B7; A1-YEVHHQKLV-B8; A1-YEVHHQKLV-B9; A1-YEVHHQKLV-B10.

A1-SEVNLDAEFR-B1; A1-SEVNLDAEFR-B2; A1-SEVNLDAEFR-B3; A1-SEVNLDAEFR-B4; A1-SEVNLDAEFR-B5; A1-SEVNLDAEFR-B6; A1-SEVNLDAEFR-B7; A1-SEVNLDAEFR-B8; A1-SEVNLDAEFR-B9; A1-SEVNLDAEFR-B10; A2-SEVNLDAEFR-B1; A2-SEVNLDAEFR-B2; A2-SEVNLDAEFR-B3; A2-SEVNLDAEFR-B4; A2-SEVNLDAEFR-B5; A1-SEVNLDAEFR-B6; A1-SEVNLDAEFR-B7; A1-SEVNLDAEFR-B8; A1-SEVNLDAEFR-B9; A1-SEVNLDAEFR-B10; A3-SEVNLDAEFR-B1; A3-SEVNLDAEFR-B2; A3-SEVNLDAEFR-B2; A3-SEVNLDAEFR-B3; A3-SEVNLDAEFR-B4; A3-SEVNLDAEFR-B5; A1-SEVNLDAEFR-B6; A1-SEVNLDAEFR-B7; A1-SEVNLDAEFR-B8; A1-SEVNLDAEFR-B9; A1-SEVNLDAEFR-B10; A4-SEVNLDAEFR-B1; A4-SEVNLDAEFR-B2; A4-SEVNLDAEFR-B2; A4-SEVNLDAEFR-B3; A4-SEVNLDAEFR-B4; A4-SEVNLDAEFR-B5; A5-SEVNLDAEFR-B1; A5-SEVNLDAEFR-B2; A5-SEVNLDAEFR-B2; A5-SEVNLDAEFR-B3; A5-SEVNLDAEFR-B4; A5-SEVNLDAEFR-B5; A1-SEVNLDAEFR-B6; A1-SEVNLDAEFR-B7; A1-SEVNLDAEFR-B8; A1-SEVNLDAEFR-B9; A1-SEVNLDAEFR-B10.

A1-LPETG-B1; A1-LPETG-B2; A1-LPETG-B3; A1-LPETG-B4; A1-LPETG-B5; A1-LPETG-B6; A1-LPETG-B7; A1-LPETG-B8; A1-LPETG-B9; A1-LPETG-B10; A2-LPETG-B1; A2-LPETG-B2; A2-LPETG-B3; A2-LPETG-B4; A2-LPETG-B5; A1-LPETG-B6; A1-LPETG-B7; A1-LPETG-B8; A1-LPETG-B9; A1-LPETG-B10; A3-LPETG-B1; A3-LPETG-B2; A3-LPETG-B2; A3-LPETG-B3; A3-LPETG-B4; A3-LPETG-B5; A1-LPETG-B6; A1-LPETG-B7; A1-LPETG-B8; A1-LPETG-B9; A1-LPETG-B10; A4-LPETG-B1; A4-LPETG-B2; A4-LPETG-B2; A4-LPETG-B3; A4-LPETG-B4; A4-LPETG-B5; A5-LPETG-B1; A5-LPETG-B2; A5-LPETG-B2; A5-LPETG-B3; A5-LPETG-B4; A5-LPETG-B5; A1-LPETG-B6; A1-LPETG-B7; A1-LPETG-B8; A1-LPETG-B9; A1-LPETG-B10.

A1-PLLAERP-B1; A1-PLLAERP-B2; A1-PLLAERP-B3; A1-PLLAERP-B4; A1-PLLAERP-B5; A1-PLLAERP-B6; A1-PLLAERP-B7; A1-PLLAERP-B8; A1-PLLAERP-B9; A1-PLLAERP-B10; A2-PLLAERP-B1; A2-PLLAERP-B2; A2-PLLAERP-B3; A2-PLLAERP-B4; A2-PLLAERP-B5; A1-PLLAERP-B6; A1-PLLAERP-B7; A1-PLLAERP-B8; A1-PLLAERP-B9; A1-PLLAERP-B10; A3-PLLAERP-B1; A3-PLLAERP-B2; A3-PLLAERP-B2; A3-PLLAERP-B3; A3-PLLAERP-B4; A3-PLLAERP-B5; A1-PLLAERP-B6; A1-PLLAERP-B7; A1-PLLAERP-B8; A1-PLLAERP-B9; A1-PLLAERP-B10; A4-PLLAERP-B1; A4-PLLAERP-B2; A4-PLLAERP-B2; A4-PLLAERP-B3; A4-PLLAERP-B4; A4-PLLAERP-B5; A5-PLLAERP-B1; A5-PLLAERP-B2; A5-PLLAERP-B2; A5-PLLAERP-B3; A5-PLLAERP-B4; A5-PLLAERP-B5; A1-PLLAERP-B6; A1-PLLAERP-B7; A1-PLLAERP-B8; A1-PLLAERP-B9; A1-PLLAERP-B10.

A1-TPLKSPPPSPR-B1; A1-TPLKSPPPSPR-B2; A1-TPLKSPPPSPR-B3; A1-TPLKSPPPSPR-B4; A1-TPLKSPPPSPR-B5; A1-TPLKSPPPSPR-B6; A1-TPLKSPPPSPR-B7; A1-TPLKSPPPSPR-B8; A1-TPLKSPPPSPR-B9; A1-TPLKSPPPSPR-B10; A2-TPLKSPPPSPR-B1; A2-TPLKSPPPSPR-B2; A2-TPLKSPPPSPR-B3; A2-TPLKSPPPSPR-B4; A2-TPLKSPPPSPR-B5; A1-TPLKSPPPSPR-B6; A1-TPLKSPPPSPR-B7; A1-TPLKSPPPSPR-B8; A1-TPLKSPPPSPR-B9; A1-TPLKSPPPSPR-B10; A3-TPLKSPPPSPR-B1; A3-TPLKSPPPSPR-B2; A3-TPLKSPPPSPR-B2; A3-TPLKSPPPSPR-B3; A3-TPLKSPPPSPR-B4; A3-TPLKSPPPSPR-B5; A1-TPLKSPPPSPR-B6; A1-TPLKSPPPSPR-B7; A1-TPLKSPPPSPR-B8; A1-TPLKSPPPSPR-B9; A1-TPLKSPPPSPR-B10; A4-TPLKSPPPSPR-B1; A4-TPLKSPPPSPR-B2; A4-TPLKSPPPSPR-B2; A4-TPLKSPPPSPR-B3; A4-TPLKSPPPSPR-B4; A4-TPLKSPPPSPR-B5; A5-TPLKSPPPSPR-B1; A5-TPLKSPPPSPR-B2; A5-TPLKSPPPSPR-B2; A5-TPLKSPPPSPR-B3; A5-TPLKSPPPSPR-B4; A5-TPLKSPPPSPR-B5; A1-TPLKSPPPSPR-B6; A1-TPLKSPPPSPR-B7; A1-TPLKSPPPSPR-B8; A1-TPLKSPPPSPR-B9; A1-TPLKSPPPSPR-B10.

A1-YVADAPV-B1; A1-YVADAPV-B2; A1-YVADAPV-B3; A1-YVADAPV-B4; A1-YVADAPV-B5; A1-YVADAPV-B6; A1-YVADAPV-B7; A1-YVADAPV-B8; A1-YVADAPV-B9; A1-YVADAPV-B10; A2-YVADAPV-B1; A2-YVADAPV-B2; A2-YVADAPV-B3; A2-YVADAPV-B4; A2-YVADAPV-B5; A1-YVADAPV-B6; A1-YVADAPV-B7; A1-YVADAPV-B8; A1-YVADAPV-B9; A1-YVADAPV-B10; A3-YVADAPV-B1; A3-YVADAPV-B2; A3-YVADAPV-B2; A3-YVADAPV-B3; A3-YVADAPV-B4; A3-YVADAPV-B5; A1-YVADAPV-B6; A1-YVADAPV-B7; A1-YVADAPV-B8; A1-YVADAPV-B9; A1-YVADAPV-B10; A4-YVADAPV-B1; A4-YVADAPV-B2; A4-YVADAPV-B2; A4-YVADAPV-B3; A4-YVADAPV-B4; A4-YVADAPV-B5; A5-YVADAPV-B1; A5-YVADAPV-B2; A5-YVADAPV-B2; A5-YVADAPV-B3; A5-YVADAPV-B4; A5-YVADAPV-B5; A1-YVADAPV-B6; A1-YVADAPV-B7; A1-YVADAPV-B8; A1-YVADAPV-B9; A1-YVADAPV-B10.

A1-ISVDRPVK-B1; A1-ISVDRPVK-B2; A1-ISVDRPVK-B3; A1-ISVDRPVK-B4; A1-ISVDRPVK-B5; A1-ISVDRPVK-B6; A1-ISVDRPVK-B7; A1-ISVDRPVK-B8; A1-ISVDRPVK-B9; A1-ISVDRPVK-B10; A2-ISVDRPVK-B1; A2-ISVDRPVK-B2; A2-ISVDRPVK-B3; A2-ISVDRPVK-B4; A2-ISVDRPVK-B5; A1-ISVDRPVK-B6; A1-ISVDRPVK-B7; A1-ISVDRPVK-B8; A1-ISVDRPVK-B9; A1-ISVDRPVK-B10; A3-ISVDRPVK-B1; A3-ISVDRPVK-B2; A3-ISVDRPVK-B2; A3-ISVDRPVK-B3; A3-ISVDRPVK-B4; A3-ISVDRPVK-B5; A1-ISVDRPVK-B6; A1-ISVDRPVK-B7; A1-ISVDRPVK-B8; A1-ISVDRPVK-B9; A1-ISVDRPVK-B10; A4-ISVDRPVK-B1; A4-ISVDRPVK-B2; A4-ISVDRPVK-B2; A4-ISVDRPVK-B3; A4-ISVDRPVK-B4; A4-ISVDRPVK-B5; A5-ISVDRPVK-B1; A5-ISVDRPVK-B2; A5-ISVDRPVK-B2; A5-ISVDRPVK-B3; A5-ISVDRPVK-B4; A5-ISVDRPVK-B5; A1-ISVDRPVK-B6; A1-ISVDRPVK-B7; A1-ISVDRPVK-B8; A1-ISVDRPVK-B9; A1-ISVDRPVK-B10.

A1-GFARGG-B1; A1-GFARGG-B2; A1-GFARGG-B3; A1-GFARGG-B4; A1-GFARGG-B5; A1-GFARGG-B6; A1-GFARGG-B7; A1-GFARGG-B8; A1-GFARGG-B9; A1-GFARGG-B10; A2-GFARGG-B1; A2-GFARGG-B2; A2-GFARGG-B3; A2-GFARGG-B4; A2-GFARGG-B5; A1-GFARGG-B6; A1-GFARGG-B7; A1-GFARGG-B8; A1-GFARGG-B9; A1-GFARGG-B10; A3-GFARGG-B1; A3-GFARGG-B2; A3-GFARGG-B2; A3-GFARGG-B3; A3-GFARGG-B4; A3-GFARGG-B5; A1-GFARGG-B6; A1-GFARGG-B7; A1-GFARGG-B8; A1-GFARGG-B9; A1-GFARGG-B10; A4-GFARGG-B1; A4-GFARGG-B2; A4-GFARGG-B2; A4-GFARGG-B3; A4-GFARGG-B4; A4-GFARGG-B5; A5-GFARGG-B1; A5-GFARGG-B2; A5-GFARGG-B2; A5-GFARGG-B3; A5-GFARGG-B4; A5-GFARGG-B5; A1-GFARGG-B6; A1-GFARGG-B7; A1-GFARGG-B8; A1-GFARGG-B9; A1-GFARGG-B10.

A1-GFLRGG-B1; A1-GFLRGG-B2; A1-GFLRGG-B3; A1-GFLRGG-B4; A1-GFLRGG-B5; A1-GFLRGG-B6; A1-GFLRGG-B7; A1-GFLRGG-B8; A1-GFLRGG-B9; A1-GFLRGG-B10; A2-GFLRGG-B1; A2-GFLRGG-B2; A2-GFLRGG-B3; A2-GFLRGG-B4; A2-GFLRGG-B5; A1-GFLRGG-B6; A1-GFLRGG-B7; A1-GFLRGG-B8; A1-GFLRGG-B9; A1-GFLRGG-B10; A3-GFLRGG-B1; A3-GFLRGG-B2; A3-GFLRGG-B2; A3-GFLRGG-B3; A3-GFLRGG-B4; A3-GFLRGG-B5; A1-GFLRGG-B6; A1-GFLRGG-B7; A1-GFLRGG-B8; A1-GFLRGG-B9; A1-GFLRGG-B10; A4-GFLRGG-B1; A4-GFLRGG-B2; A4-GFLRGG-B2; A4-GFLRGG-B3; A4-GFLRGG-B4; A4-GFLRGG-B5; A5-GFLRGG-B1; A5-GFLRGG-B2; A5-GFLRGG-B2; A5-GFLRGG-B3; A5-GFLRGG-B4; A5-GFLRGG-B5; A1-GFLRGG-B6; A1-GFLRGG-B7; A1-GFLRGG-B8; A1-GFLRGG-B9; A1-GFLRGG-B10.

A1-AGLQRGG-B1; A1-AGLQRGG-B2; A1-AGLQRGG-B3; A1-AGLQRGG-B4; A1-AGLQRGG-B5; A1-AGLQRGG-B6; A1-AGLQRGG-B7; A1-AGLQRGG-B8; A1-AGLQRGG-B9; A1-AGLQRGG-B10; A2-AGLQRGG-B1; A2-AGLQRGG-B2; A2-AGLQRGG-B3; A2-AGLQRGG-B4; A2-AGLQRGG-B5; A1-AGLQRGG-B6; A1-AGLQRGG-B7; A1-AGLQRGG-B8; A1-AGLQRGG-B9; A1-AGLQRGG-B10; A3-AGLQRGG-B1; A3-AGLQRGG-B2; A3-AGLQRGG-B2; A3-AGLQRGG-B3; A3-AGLQRGG-B4; A3-AGLQRGG-B5; A1-AGLQRGG-B6; A1-AGLQRGG-B7; A1-AGLQRGG-B8; A1-AGLQRGG-B9; A1-AGLQRGG-B10; A4-AGLQRGG-B1; A4-AGLQRGG-B2; A4-AGLQRGG-B2; A4-AGLQRGG-B3; A4-AGLQRGG-B4; A4-AGLQRGG-B5; A5-AGLQRGG-B1; A5-AGLQRGG-B2; A5-AGLQRGG-B2; A5-AGLQRGG-B3; A5-AGLQRGG-B4; A5-AGLQRGG-B5; A1-AGLQRGG-B6; A1-AGLQRGG-B7; A1-AGLQRGG-B8; A1-AGLQRGG-B9; A1-AGLQRGG-B10.

A1-LESDYFGK-B1; A1-LESDYFGK-B2; A1-LESDYFGK-B3; A1-LESDYFGK-B4; A1-LESDYFGK-B5; A1-LESDYFGK-B6; A1-LESDYFGK-B7; A1-LESDYFGK-B8; A1-LESDYFGK-B9; A1-LESDYFGK-B10; A2-LESDYFGK-B1; A2-LESDYFGK-B2; A2-LESDYFGK-B3; A2-LESDYFGK-B4; A2-LESDYFGK-B5; A1-LESDYFGK-B6; A1-LESDYFGK-B7; A1-LESDYFGK-B8; A1-LESDYFGK-B9; A1-LESDYFGK-B10; A3-LESDYFGK-B1; A3-LESDYFGK-B2; A3-LESDYFGK-B2; A3-LESDYFGK-B3; A3-LESDYFGK-B4; A3-LESDYFGK-B5; A1-LESDYFGK-B6; A1-LESDYFGK-B7; A1-LESDYFGK-B8; A1-LESDYFGK-B9; A1-LESDYFGK-B10; A4-LESDYFGK-B1; A4-LESDYFGK-B2; A4-LESDYFGK-B2; A4-LESDYFGK-B3; A4-LESDYFGK-B4; A4-LESDYFGK-B5; A5-LESDYFGK-B1; A5-LESDYFGK-B2; A5-LESDYFGK-B2; A5-LESDYFGK-B3; A5-LESDYFGK-B4; A5-LESDYFGK-B5; A1-LESDYFGK-B6; A1-LESDYFGK-B7; A1-LESDYFGK-B8; A1-LESDYFGK-B9; A1-LESDYFGK-B10.

A1-PFDLLDFN-B1; A1-PFDLLDFN-B2; A1-PFDLLDFN-B3; A1-PFDLLDFN-B4; A1-PFDLLDFN-B5; A1-PFDLLDFN-B6; A1-PFDLLDFN-B7; A1-PFDLLDFN-B8; A1-PFDLLDFN-B9; A1-PFDLLDFN-B10; A2-PFDLLDFN-B1; A2-PFDLLDFN-B2; A2-PFDLLDFN-B3; A2-PFDLLDFN-B4; A2-PFDLLDFN-B5; A1-PFDLLDFN-B6; A1-PFDLLDFN-B7; A1-PFDLLDFN-B8; A1-PFDLLDFN-B9; A1-PFDLLDFN-B10; A3-PFDLLDFN-B1; A3-PFDLLDFN-B2; A3-PFDLLDFN-B2; A3-PFDLLDFN-B3; A3-PFDLLDFN-B4; A3-PFDLLDFN-B5; A1-PFDLLDFN-B6; A1-PFDLLDFN-B7; A1-PFDLLDFN-B8; A1-PFDLLDFN-B9; A1-PFDLLDFN-B10; A4-PFDLLDFN-B1; A4-PFDLLDFN-B2; A4-PFDLLDFN-B2; A4-PFDLLDFN-B3; A4-PFDLLDFN-B4; A4-PFDLLDFN-B5; A5-PFDLLDFN-B1; A5-PFDLLDFN-B2; A5-PFDLLDFN-B2; A5-PFDLLDFN-B3; A5-PFDLLDFN-B4; A5-PFDLLDFN-B5; A1-PFDLLDFN-B6; A1-PFDLLDFN-B7; A1-PFDLLDFN-B8; A1-PFDLLDFN-B9; A1-PFDLLDFN-B10.

A1-GDEVDGAK-B1; A1-GDEVDGAK-B2; A1-GDEVDGAK-B3; A1-GDEVDGAK-B4; A1-GDEVDGAK-B5; A1-GDEVDGAK-B6; A1-GDEVDGAK-B7; A1-GDEVDGAK-B8; A1-GDEVDGAK-B9; A1-GDEVDGAK-B10; A2-GDEVDGAK-B1; A2-GDEVDGAK-B2; A2-GDEVDGAK-B3; A2-GDEVDGAK-B4; A2-GDEVDGAK-B5; A1-GDEVDGAK-B6; A1-GDEVDGAK-B7; A1-GDEVDGAK-B8; A1-GDEVDGAK-B9; A1-GDEVDGAK-B10; A3-GDEVDGAK-B1; A3-GDEVDGAK-B2; A3-GDEVDGAK-B2; A3-GDEVDGAK-B3; A3-GDEVDGAK-B4; A3-GDEVDGAK-B5; A1-GDEVDGAK-B6; A1-GDEVDGAK-B7; A1-GDEVDGAK-B8; A1-GDEVDGAK-B9; A1-GDEVDGAK-B10; A4-GDEVDGAK-B1; A4-GDEVDGAK-B2; A4-GDEVDGAK-B2; A4-GDEVDGAK-B3; A4-GDEVDGAK-B4; A4-GDEVDGAK-B5; A5-GDEVDGAK-B1; A5-GDEVDGAK-B2; A5-GDEVDGAK-B2; A5-GDEVDGAK-B3; A5-GDEVDGAK-B4; A5-GDEVDGAK-B5; A1-GDEVDGAK-B6; A1-GDEVDGAK-B7; A1-GDEVDGAK-B8; A1-GDEVDGAK-B9; A1-GDEVDGAK-B10.

A1-NDTDANPR-B1; A1-NDTDANPR-B2; A1-NDTDANPR-B3; A1-NDTDANPR-B4; A1-NDTDANPR-B5; A1-NDTDANPR-B6; A1-NDTDANPR-B7; A1-NDTDANPR-B8; A1-NDTDANPR-B9; A1-NDTDANPR-B10; A2-NDTDANPR-B1; A2-NDTDANPR-B2; A2-NDTDANPR-B3; A2-NDTDANPR-B4; A2-NDTDANPR-B5; A1-NDTDANPR-B6; A1-NDTDANPR-B7; A1-NDTDANPR-B8; A1-NDTDANPR-B9; A1-NDTDANPR-B10; A3-NDTDANPR-B1; A3-NDTDANPR-B2; A3-NDTDANPR-B2; A3-NDTDANPR-B3; A3-NDTDANPR-B4; A3-NDTDANPR-B5; A1-NDTDANPR-B6; A1-NDTDANPR-B7; A1-NDTDANPR-B8; A1-NDTDANPR-B9; A1-NDTDANPR-B10; A4-NDTDANPR-B1; A4-NDTDANPR-B2; A4-NDTDANPR-B2; A4-NDTDANPR-B3; A4-NDTDANPR-B4; A4-NDTDANPR-B5; A5-NDTDANPR-B1; A5-NDTDANPR-B2; A5-NDTDANPR-B2; A5-NDTDANPR-B3; A5-NDTDANPR-B4; A5-NDTDANPR-B5; A1-NDTDANPR-B6; A1-NDTDANPR-B7; A1-NDTDANPR-B8; A1-NDTDANPR-B9; A1-NDTDANPR-B10.

A1-IQADSGPI-B1; A1-IQADSGPI-B2; A1-IQADSGPI-B3; A1-IQADSGPI-B4; A1-IQADSGPI-B5; A1-IQADSGPI-B6; A1-IQADSGPI-B7; A1-IQADSGPI-B8; A1-IQADSGPI-B9; A1-IQADSGPI-B10; A2-IQADSGPI-B1; A2-IQADSGPI-B2; A2-IQADSGPI-B3; A2-IQADSGPI-B4; A2-IQADSGPI-B5; A1-IQADSGPI-B6; A1-IQADSGPI-B7; A1-IQADSGPI-B8; A1-IQADSGPI-B9; A1-IQADSGPI-B10; A3-IQADSGPI-B1; A3-IQADSGPI-B2; A3-IQADSGPI-B2; A3-IQADSGPI-B3; A3-IQADSGPI-B4; A3-IQADSGPI-B5; A1-IQADSGPI-B6; A1-IQADSGPI-B7; A1-IQADSGPI-B8; A1-IQADSGPI-B9; A1-IQADSGPI-B10; A4-IQADSGPI-B1; A4-IQADSGPI-B2; A4-IQADSGPI-B2; A4-IQADSGPI-B3; A4-IQADSGPI-B4; A4-IQADSGPI-B5; A5-IQADSGPI-B1; A5-IQADSGPI-B2; A5-IQADSGPI-B2; A5-IQADSGPI-B3; A5-IQADSGPI-B4; A5-IQADSGPI-B5; A1-IQADSGPI-B6; A1-IQADSGPI-B7; A1-IQADSGPI-B8; A1-IQADSGPI-B9; A1-IQADSGPI-B10.

A1-LQLDCVAV-B1; A1-LQLDCVAV-B2; A1-LQLDCVAV-B3; A1-LQLDCVAV-B4; A1-LQLDCVAV-B5; A1-LQLDCVAV-B6; A1-LQLDCVAV-B7; A1-LQLDCVAV-B8; A1-LQLDCVAV-B9; A1-LQLDCVAV-B10; A2-LQLDCVAV-B1; A2-LQLDCVAV-B2; A2-LQLDCVAV-B3; A2-LQLDCVAV-B4; A2-LQLDCVAV-B5; A1-LQLDCVAV-B6; A1-LQLDCVAV-B7; A1-LQLDCVAV-B8; A1-LQLDCVAV-B9; A1-LQLDCVAV-B10; A3-LQLDCVAV-B1; A3-LQLDCVAV-B2; A3-LQLDCVAV-B2; A3-LQLDCVAV-B3; A3-LQLDCVAV-B4; A3-LQLDCVAV-B5; A1-LQLDCVAV-B6; A1-LQLDCVAV-B7; A1-LQLDCVAV-B8; A1-LQLDCVAV-B9; A1-LQLDCVAV-B10; A4-LQLDCVAV-B1; A4-LQLDCVAV-B2; A4-LQLDCVAV-B2; A4-LQLDCVAV-B3; A4-LQLDCVAV-B4; A4-LQLDCVAV-B5; A5-LQLDCVAV-B1; A5-LQLDCVAV-B2; A5-LQLDCVAV-B2; A5-LQLDCVAV-B3; A5-LQLDCVAV-B4; A5-LQLDCVAV-B5; A1-LQLDCVAV-B6; A1-LQLDCVAV-B7; A1-LQLDCVAV-B8; A1-LQLDCVAV-B9; A1-LQLDCVAV-B10.

A1-LLVYGG-B1; A1-LLVYGG-B2; A1-LLVYGG-B3; A1-LLVYGG-B4; A1-LLVYGG-B5; A1-LLVYGG-B6; A1-LLVYGG-B7; A1-LLVYGG-B8; A1-LLVYGG-B9; A1-LLVYGG-B10; A2-LLVYGG-B1; A2-LLVYGG-B2; A2-LLVYGG-B3; A2-LLVYGG-B4; A2-LLVYGG-B5; A1-LLVYGG-B6; A1-LLVYGG-B7; A1-LLVYGG-B8; A1-LLVYGG-B9; A1-LLVYGG-B10; A3-LLVYGG-B1; A3-LLVYGG-B2; A3-LLVYGG-B2; A3-LLVYGG-B3; A3-LLVYGG-B4; A3-LLVYGG-B5; A1-LLVYGG-B6; A1-LLVYGG-B7; A1-LLVYGG-B8; A1-LLVYGG-B9; A1-LLVYGG-B10; A4-LLVYGG-B1; A4-LLVYGG-B2; A4-LLVYGG-B2; A4-LLVYGG-B3; A4-LLVYGG-B4; A4-LLVYGG-B5; A5-LLVYGG-B1; A5-LLVYGG-B2; A5-LLVYGG-B2; A5-LLVYGG-B3; A5-LLVYGG-B4; A5-LLVYGG-B5; A1-LLVYGG-B6; A1-LLVYGG-B7; A1-LLVYGG-B8; A1-LLVYGG-B9; A1-LLVYGG-B10.

A1-YDAKGD-B1; A1-YDAKGD-B2; A1-YDAKGD-B3; A1-YDAKGD-B4; A1-YDAKGD-B5; A1-YDAKGD-B6; A1-YDAKGD-B7; A1-YDAKGD-B8; A1-YDAKGD-B9; A1-YDAKGD-B10; A2-YDAKGD-B1; A2-YDAKGD-B2; A2-YDAKGD-B3; A2-YDAKGD-B4; A2-YDAKGD-B5; A1-YDAKGD-B6; A1-YDAKGD-B7; A1-YDAKGD-B8; A1-YDAKGD-B9; A1-YDAKGD-B10; A3-YDAKGD-B1; A3-YDAKGD-B2; A3-YDAKGD-B2; A3-YDAKGD-B 3; A3-YDAKGD-B4; A3-YDAKGD-B5; A1-YDAKGD-B6; A1-YDAKGD-B7; A1-YDAKGD-B8; A1-YDAKGD-B9; A1-YDAKGD-B10; A4-YDAKGD-B1; A4-YDAKGD-B2; A4-YDAKGD-B2; A4-YDAKGD-B3; A4-YDAKGD-B4; A4-YDAKGD-B5; A5-YDAKGD-B1; A5-YDAKGD-B2; A5-YDAKGD-B2; A5-YDAKGD-B3; A5-YDAKGD-B4; A5-YDAKGD-B5; A1-YDAKGD-B6; A1-YDAKGD-B7; A1-YDAKGD-B8; A1-YDAKGD-B9; A1-YDAKGD-B10.

A1-GKPILFFRL-B1; A1-GKPILFFRL-B2; A1-GKPILFFRL-B3; A1-GKPILFFRL-B4; A1-GKPILFFRL-B5; A1-GKPILFFRL-B6; A1-GKPILFFRL-B7; A1-GKPILFFRL-B8; A1-GKPILFFRL-B9; A1-GKPILFFRL-B10; A2-GKPILFFRL-B1; A2-GKPILFFRL-B2; A2-GKPILFFRL-B3; A2-GKPILFFRL-B4; A2-GKPILFFRL-B5; A1-GKPILFFRL-B6; A1-GKPILFFRL-B7; A1-GKPILFFRL-B8; A1-GKPILFFRL-B9; A1-GKPILFFRL-B10; A3-GKPILFFRL-B1; A3-GKPILFFRL-B2; A3-GKPILFFRL-B2; A3-GKPILFFRL-B3; A3-GKPILFFRL-B4; A3-GKPILFFRL-B5; A1-GKPILFFRL-B6; A1-GKPILFFRL-B7; A1-GKPILFFRL-B8; A1-GKPILFFRL-B9; A1-GKPILFFRL-B10; A4-GKPILFFRL-B1; A4-GKPILFFRL-B2; A4-GKPILFFRL-B2; A4-GKPILFFRL-B3; A4-GKPILFFRL-B4; A4-GKPILFFRL-B5; A5-GKPILFFRL-B1; A5-GKPILFFRL-B2; A5-GKPILFFRL-B2; A5-GKPILFFRL-B3; A5-GKPILFFRL-B4; A5-GKPILFFRL-B5; A1-GKPILFFRL-B6; A1-GKPILFFRL-B7; A1-GKPILFFRL-B8; A1-GKPILFFRL-B9; A1-GKPILFFRL-B10.

A1-GKPIIFFRL-B1; A1-GKPIIFFRL-B2; A1-GKPIIFFRL-B3; A1-GKPIIFFRL-B4; A1-GKPIIFFRL-B5; A1-GKPIIFFRL-B6; A1-GKPIIFFRL-B7; A1-GKPIIFFRL-B8; A1-GKPIIFFRL-B9; A1-GKPIIFFRL-B10; A2-GKPIIFFRL-B1; A2-GKPIIFFRL-B2; A2-GKPIIFFRL-B3; A2-GKPIIFFRL-B4; A2-GKPIIFFRL-B5; A1-GKPIIFFRL-B6; A1-GKPIIFFRL-B7; A1-GKPIIFFRL-B8; A1-GKPIIFFRL-B9; A1-GKPIIFFRL-B10; A3-GKPIIFFRL-B1; A3-GKPIIFFRL-B2; A3-GKPIIFFRL-B2; A3-GKPIIFFRL-B3; A3-GKPIIFFRL-B4; A3-GKPIIFFRL-B5; A1-GKPIIFFRL-B6; A1-GKPIIFFRL-B7; A1-GKPIIFFRL-B8; A1-GKPIIFFRL-B9; A1-GKPIIFFRL-B10; A4-GKPIIFFRL-B1; A4-GKPIIFFRL-B2; A4-GKPIIFFRL-B2; A4-GKPIIFFRL-B3; A4-GKPIIFFRL-B4; A4-GKPIIFFRL-B5; A5-GKPIIFFRL-B1; A5-GKPIIFFRL-B2; A5-GKPIIFFRL-B2; A5-GKPIIFFRL-B3; A5-GKPIIFFRL-B4; A5-GKPIIFFRL-B5; A1-GKPIIFFRL-B6; A1-GKPIIFFRL-B7; A1-GKPIIFFRL-B8; A1-GKPIIFFRL-B9; A1-GKPIIFFRL-B10.

A1-AAFFAA-B1; A1-AAFFAA-B2; A1-AAFFAA-B3; A1-AAFFAA-B4; A1-AAFFAA-B5; A1-AAFFAA-B6; A1-AAFFAA-B7; A1-AAFFAA-B8; A1-AAFFAA-B9; A1-AAFFAA-B10; A2-AAFFAA-B1; A2-AAFFAA-B2; A2-AAFFAA-B3; A2-AAFFAA-B4; A2-AAFFAA-B5; A1-AAFFAA-B6; A1-AAFFAA-B7; A1-AAFFAA-B8; A1-AAFFAA-B9; A1-AAFFAA-B10; A3-AAFFAA-B1; A3-AAFFAA-B2; A3-AAFFAA-B2; A3-AAFFAA-B3; A3-AAFFAA-B4; A3-AAFFAA-B5; A1-AAFFAA-B6; A1-AAFFAA-B7; A1-AAFFAA-B8; A1-AAFFAA-B9; A1-AAFFAA-B10; A4-AAFFAA-B1; A4-AAFFAA-B2; A4-AAFFAA-B2; A4-AAFFAA-B3; A4-AAFFAA-B4; A4-AAFFAA-B5; A5-AAFFAA-B1; A5-AAFFAA-B2; A5-AAFFAA-B2; A5-AAFFAA-B3; A5-AAFFAA-B4; A5-AAFFAA-B5; A1-AAFFAA-B6; A1-AAFFAA-B7; A1-AAFFAA-B8; A1-AAFFAA-B9; A1-AAFFAA-B10.

A1-GKPILFFRL-B1; A1-GKPILFFRL-B2; A1-GKPILFFRL-B3; A1-GKPILFFRL-B4; A1-GKPILFFRL-B5; A1-GKPILFFRL-B6; A1-GKPILFFRL-B7; A1-GKPILFFRL-B8; A1-GKPILFFRL-B9; A1-GKPILFFRL-B10; A2-GKPILFFRL-B1; A2-GKPILFFRL-B2; A2-GKPILFFRL-B3; A2-GKPILFFRL-B4; A2-GKPILFFRL-B5; A1-GKPILFFRL-B6; A1-GKPILFFRL-B7; A1-GKPILFFRL-B8; A1-GKPILFFRL-B9; A1-GKPILFFRL-B10; A3-GKPILFFRL-B1; A3-GKPILFFRL-B2; A3-GKPILFFRL-B2; A3-GKPILFFRL-B3; A3-GKPILFFRL-B4; A3-GKPILFFRL-B5; A1-GKPILFFRL-B6; A1-GKPILFFRL-B7; A1-GKPILFFRL-B8; A1-GKPILFFRL-B9; A1-GKPILFFRL-B10; A4-GKPILFFRL-B1; A4-GKPILFFRL-B2; A4-GKPILFFRL-B2; A4-GKPILFFRL-B3; A4-GKPILFFRL-B4; A4-GKPILFFRL-B5; A5-GKPILFFRL-B1; A5-GKPILFFRL-B2; A5-GKPILFFRL-B2; A5-GKPILFFRL-B3; A5-GKPILFFRL-B4; A5-GKPILFFRL-B5; A1-GKPILFFRL-B6; A1-GKPILFFRL-B7; A1-GKPILFFRL-B8; A1-GKPILFFRL-B9; A1-GKPILFFRL-B10.

A1-FVT-Gnf-SW-B1; A1-FVT-Gnf-SW-B2; A1-FVT-Gnf-SW-B3; A1-FVT-Gnf-SW-B4; A1-FVT-Gnf-SW-B5; A1-FVT-Gnf-SW-B6; A1-FVT-Gnf-SW-B7; A1-FVT-Gnf-SW-B8; A1-FVT-Gnf-SW-B9; A1-FVT-Gnf-SW-B10; A2-FVT-Gnf-SW-B1; A2-FVT-Gnf-SW-B2; A2-FVT-Gnf-SW-B3; A2-FVT-Gnf-SW-B4; A2-FVT-Gnf-SW-B5; A1-FVT-Gnf-SW-B6; A1-FVT-Gnf-SW-B7; A1-FVT-Gnf-SW-B8; A1-FVT-Gnf-SW-B9; A1-FVT-Gnf-SW-B10; A3-FVT-Gnf-SW-B1; A3-FVT-Gnf-SW-B2; A3-FVT-Gnf-SW-B2; A3-FVT-Gnf-SW-B3; A3-FVT-Gnf-SW-B4; A3-FVT-Gnf-SW-B5; A1-FVT-Gnf-SW-B6; A1-FVT-Gnf-SW-B7; A1-FVT-Gnf-SW-B8; A1-FVT-Gnf-SW-B9; A1-FVT-Gnf-SW-B10; A4-FVT-Gnf-SW-B1; A4-FVT-Gnf-SW-B2; A4-FVT-Gnf-SW-B2; A4-FVT-Gnf-SW-B3; A4-FVT-Gnf-SW-B4; A4-FVT-Gnf-SW-B5; A5-FVT-Gnf-SW-B1; A5-FVT-Gnf-SW-B2; A5-FVT-Gnf-SW-B2; A5-FVT-Gnf-SW-B3; A5-FVT-Gnf-SW-B4; A5-FVT-Gnf-SW-B5; A1-FVT-Gnf-SW-B6; A1-FVT-Gnf-SW-B7; A1-FVT-Gnf-SW-B8; A1-FVT-Gnf-SW-B9; A1-FVT-Gnf-SW-B10.

A1-DTDLYDYY-B1; A1-DTDLYDYY-B2; A1-DTDLYDYY-B3; A1-DTDLYDYY-B4; A1-DTDLYDYY-B5; A1-DTDLYDYY-B6; A1-DTDLYDYY-B7; A1-DTDLYDYY-B8; A1-DTDLYDYY-B9; A1-DTDLYDYY-B10; A2-DTDLYDYY-B1; A2-DTDLYDYY-B2; A2-DTDLYDYY-B3; A2-DTDLYDYY-B4; A2-DTDLYDYY-B5; A1-DTDLYDYY-B6; A1-DTDLYDYY-B7; A1-DTDLYDYY-B8; A1-DTDLYDYY-B9; A1-DTDLYDYY-B10; A3-DTDLYDYY-B1; A3-DTDLYDYY-B2; A3-DTDLYDYY-B2; A3-DTDLYDYY-B3; A3-DTDLYDYY-B4; A3-DTDLYDYY-B5; A1-DTDLYDYY-B6; A1-DTDLYDYY-B7; A1-DTDLYDYY-B8; A1-DTDLYDYY-B9; A1-DTDLYDYY-B10; A4-DTDLYDYY-B1; A4-DTDLYDYY-B2; A4-DTDLYDYY-B2; A4-DTDLYDYY-B3; A4-DTDLYDYY-B4; A4-DTDLYDYY-B5; A5-DTDLYDYY-B1; A5-DTDLYDYY-B2; A5-DTDLYDYY-B2; A5-DTDLYDYY-B3; A5-DTDLYDYY-B4; A5-DTDLYDYY-B5; A1-DTDLYDYY-B6; A1-DTDLYDYY-B7; A1-DTDLYDYY-B8; A1-DTDLYDYY-B9; A1-DTDLYDYY-B10.

A1-HPGGPQ-B1; A1-HPGGPQ-B2; A1-HPGGPQ-B3; A1-HPGGPQ-B4; A1-HPGGPQ-B5; A1-HPGGPQ-B6; A1-HPGGPQ-B7; A1-HPGGPQ-B8; A1-HPGGPQ-B9; A1-HPGGPQ-B10; A2-HPGGPQ-B1; A2-HPGGPQ-B2; A2-HPGGPQ-B3; A2-HPGGPQ-B4; A2-HPGGPQ-B5; A1-HPGGPQ-B6; A1-HPGGPQ-B7; A1-HPGGPQ-B8; A1-HPGGPQ-B9; A1-HPGGPQ-B10; A3-HPGGPQ-B1; A3-HPGGPQ-B2; A3-HPGGPQ-B2; A3-HPGGPQ-B3; A3-HPGGPQ-B4; A3-HPGGPQ-B5; A1-HPGGPQ-B6; A1-HPGGPQ-B7; A1-HPGGPQ-B8; A1-HPGGPQ-B9; A1-HPGGPQ-B10; A4-HPGGPQ-B1; A4-HPGGPQ-B2; A4-HPGGPQ-B2; A4-HPGGPQ-B3; A4-HPGGPQ-B4; A4-HPGGPQ-B5; A5-HPGGPQ-B1; A5-HPGGPQ-B2; A5-HPGGPQ-B2; A5-HPGGPQ-B3; A5-HPGGPQ-B4; A5-HPGGPQ-B5; A1-HPGGPQ-B6; A1-HPGGPQ-B7; A1-HPGGPQ-B8; A1-HPGGPQ-B9; A1-HPGGPQ-B10.

A1-EKARVLAEAA-B1; A1-EKARVLAEAA-B2; A1-EKARVLAEAA-B3; A1-EKARVLAEAA-B4; A1-EKARVLAEAA-B5; A1-EKARVLAEAA-B6; A1-EKARVLAEAA-B7; A1-EKARVLAEAA-B8; A1-EKARVLAEAA-B9; A1-EKARVLAEAA-B10; A2-EKARVLAEAA-B1; A2-EKARVLAEAA-B2; A2-EKARVLAEAA-B3; A2-EKARVLAEAA-B4; A2-EKARVLAEAA-B5; A1-EKARVLAEAA-B6; A1-EKARVLAEAA-B7; A1-EKARVLAEAA-B8; A1-EKARVLAEAA-B9; A1-EKARVLAEAA-B10; A3-EKARVLAEAA-B1; A3-EKARVLAEAA-B2; A3-EKARVLAEAA-B2; A3-EKARVLAEAA-B3; A3-EKARVLAEAA-B4; A3-EKARVLAEAA-B5; A1-EKARVLAEAA-B6; A1-EKARVLAEAA-B7; A1-EKARVLAEAA-B8; A1-EKARVLAEAA-B9; A1-EKARVLAEAA-B10; A4-EKARVLAEAA-B1; A4-EKARVLAEAA-B2; A4-EKARVLAEAA-B2; A4-EKARVLAEAA-B3; A4-EKARVLAEAA-B4; A4-EKARVLAEAA-B5; A5-EKARVLAEAA-B1; A5-EKARVLAEAA-B2; A5-EKARVLAEAA-B2; A5-EKARVLAEAA-B3; A5-EKARVLAEAA-B4; A5-EKARVLAEAA-B5; A1-EKARVLAEAA-B6; A1-EKARVLAEAA-B7; A1-EKARVLAEAA-B8; A1-EKARVLAEAA-B9; A1-EKARVLAEAA-B10.

A1-EKARVLAEAA-B1; A1-EKARVLAEAA-B2; A1-EKARVLAEAA-B3; A1-EKARVLAEAA-B4; A1-EKARVLAEAA-B5; A1-EKARVLAEAA-B6; A1-EKARVLAEAA-B7; A1-EKARVLAEAA-B8; A1-EKARVLAEAA-B9; A1-EKARVLAEAA-B10; A2-EKARVLAEAA-B1; A2-EKARVLAEAA-B2; A2-EKARVLAEAA-B3; A2-EKARVLAEAA-B4; A2-EKARVLAEAA-B5; A1-EKARVLAEAA-B6; A1-EKARVLAEAA-B7; A1-EKARVLAEAA-B8; A1-EKARVLAEAA-B9; A1-EKARVLAEAA-B10; A3-EKARVLAEAA-B1; A3-EKARVLAEAA-B2; A3-EKARVLAEAA-B2; A3-EKARVLAEAA-B3; A3-EKARVLAEAA-B4; A3-EKARVLAEAA-B5; A1-EKARVLAEAA-B6; A1-EKARVLAEAA-B7; A1-EKARVLAEAA-B8; A1-EKARVLAEAA-B9; A1-EKARVLAEAA-B10; A4-EKARVLAEAA-B1; A4-EKARVLAEAA-B2; A4-EKARVLAEAA-B2; A4-EKARVLAEAA-B3; A4-EKARVLAEAA-B4; A4-EKARVLAEAA-B5; A5-EKARVLAEAA-B1; A5-EKARVLAEAA-B2; A5-EKARVLAEAA-B2; A5-EKARVLAEAA-B3; A5-EKARVLAEAA-B4; A5-EKARVLAEAA-B5; A1-EKARVLAEAA-B6; A1-EKARVLAEAA-B7; A1-EKARVLAEAA-B8; A1-EKARVLAEAA-B9; A1-EKARVLAEAA-B10.

A1-APVK-B1; A1-APVK-B2; A1-APVK-B3; A1-APVK-B4; A1-APVK-B5; A1-APVK-B6; A1-APVK-B7; A1-APVK-B8; A1-APVK-B9; A1-APVK-B10; A2-APVK-B1; A2-APVK-B2; A2-APVK-B3; A2-APVK-B4; A2-APVK-B5; A1-APVK-B6; A1-APVK-B7; A1-APVK-B8; A1-APVK-B9; A1-APVK-B10; A3-APVK-B1; A3-APVK-B2; A3-APVK-B2; A3-APVK-B3; A3-APVK-B4; A3-APVK-B5; A1-APVK-B6; A1-APVK-B7; A1-APVK-B8; A1-APVK-B9; A1-APVK-B10; A4-APVK-B1; A4-APVK-B2; A4-APVK-B2; A4-APVK-B3; A4-APVK-B4; A4-APVK-B5; A5-APVK-B1; A5-APVK-B2; A5-APVK-B2; A5-APVK-B3; A5-APVK-B4; A5-APVK-B5; A1-APVK-B6; A1-APVK-B7; A1-APVK-B8; A1-APVK-B9; A1-APVK-B10.

A1-LAYGLRSK-B1; A1-LAYGLRSK-B2; A1-LAYGLRSK-B3; A1-LAYGLRSK-B4; A1-LAYGLRSK-B5; A1-LAYGLRSK-B6; A1-LAYGLRSK-B7; A1-LAYGLRSK-B8; A1-LAYGLRSK-B9; A1-LAYGLRSK-B10; A2-LAYGLRSK-B1; A2-LAYGLRSK-B2; A2-LAYGLRSK-B3; A2-LAYGLRSK-B4; A2-LAYGLRSK-B5; A1-LAYGLRSK-B6; A1-LAYGLRSK-B7; A1-LAYGLRSK-B8; A1-LAYGLRSK-B9; A1-LAYGLRSK-B10; A3-LAYGLRSK-B1; A3-LAYGLRSK-B2; A3-LAYGLRSK-B2; A3-LAYGLRSK-B3; A3-LAYGLRSK-B4; A3-LAYGLRSK-B5; A1-LAYGLRSK-B6; A1-LAYGLRSK-B7; A1-LAYGLRSK-B8; A1-LAYGLRSK-B9; A1-LAYGLRSK-B10; A4-LAYGLRSK-B1; A4-LAYGLRSK-B2; A4-LAYGLRSK-B2; A4-LAYGLRSK-B3; A4-LAYGLRSK-B4; A4-LAYGLRSK-B5; A5-LAYGLRSK-B1; A5-LAYGLRSK-B2; A5-LAYGLRSK-B2; A5-LAYGLRSK-B3; A5-LAYGLRSK-B4; A5-LAYGLRSK-B5; A1-LAYGLRSK-B6; A1-LAYGLRSK-B7; A1-LAYGLRSK-B8; A1-LAYGLRSK-B9; A1-LAYGLRSK-B10.

A1-SLGRKIQIQ-B1; A1-SLGRKIQIQ-B2; A1-SLGRKIQIQ-B3; A1-SLGRKIQIQ-B4; A1-SLGRKIQIQ-B5; A1-SLGRKIQIQ-B6; A1-SLGRKIQIQ-B7; A1-SLGRKIQIQ-B8; A1-SLGRKIQIQ-B9; A1-SLGRKIQIQ-B10; A2-SLGRKIQIQ-B1; A2-SLGRKIQIQ-B2; A2-SLGRKIQIQ-B3; A2-SLGRKIQIQ-B4; A2-SLGRKIQIQ-B5; A1-SLGRKIQIQ-B6; A1-SLGRKIQIQ-B7; A1-SLGRKIQIQ-B8; A1-SLGRKIQIQ-B9; A1-SLGRKIQIQ-B10; A3-SLGRKIQIQ-B1; A3-SLGRKIQIQ-B2; A3-SLGRKIQIQ-B2; A3-SLGRKIQIQ-B3; A3-SLGRKIQIQ-B4; A3-SLGRKIQIQ-B5; A1-SLGRKIQIQ-B6; A1-SLGRKIQIQ-B7; A1-SLGRKIQIQ-B8; A1-SLGRKIQIQ-B9; A1-SLGRKIQIQ-B10; A4-SLGRKIQIQ-B1; A4-SLGRKIQIQ-B2; A4-SLGRKIQIQ-B2; A4-SLGRKIQIQ-B3; A4-SLGRKIQIQ-B4; A4-SLGRKIQIQ-B5; A5-SLGRKIQIQ-B1; A5-SLGRKIQIQ-B2; A5-SLGRKIQIQ-B2; A5-SLGRKIQIQ-B3; A5-SLGRKIQIQ-B4; A5-SLGRKIQIQ-B5; A1-SLGRKIQIQ-B6; A1-SLGRKIQIQ-B7; A1-SLGRKIQIQ-B8; A1-SLGRKIQIQ-B9; A1-SLGRKIQIQ-B10.

A1-RGVVNASSRL-B1; A1-RGVVNASSRL-B2; A1-RGVVNASSRL-B3; A1-RGVVNASSRL-B4; A1-RGVVNASSRL-B5; A1-RGVVNASSRL-B6; A1-RGVVNASSRL-B7; A1-RGVVNASSRL-B8; A1-RGVVNASSRL-B9; A1-RGVVNASSRL-B10; A2-RGVVNASSRL-B1; A2-RGVVNASSRL-B2; A2-RGVVNASSRL-B3; A2-RGVVNASSRL-B4; A2-RGVVNASSRL-B5; A1-RGVVNASSRL-B6; A1-RGVVNASSRL-B7; A1-RGVVNASSRL-B8; A1-RGVVNASSRL-B9; A1-RGVVNASSRL-B10; A3-RGVVNASSRL-B1; A3-RGVVNASSRL-B2; A3-RGVVNASSRL-B2; A3-RGVVNASSRL-B3; A3-RGVVNASSRL-B4; A3-RGVVNASSRL-B5; A1-RGVVNASSRL-B6; A1-RGVVNASSRL-B7; A1-RGVVNASSRL-B8; A1-RGVVNASSRL-B9; A1-RGVVNASSRL-B10; A4-RGVVNASSRL-B1; A4-RGVVNASSRL-B2; A4-RGVVNASSRL-B2; A4-RGVVNASSRL-B3; A4-RGVVNASSRL-B4; A4-RGVVNASSRL-B5; A5-RGVVNASSRL-B1; A5-RGVVNASSRL-B2; A5-RGVVNASSRL-B2; A5-RGVVNASSRL-B3; A5-RGVVNASSRL-B4; A5-RGVVNASSRL-B5; A1-RGVVNASSRL-B6; A1-RGVVNASSRL-B7; A1-RGVVNASSRL-B8; A1-RGVVNASSRL-B9; A1-RGVVNASSRL-B10.

A1-RPPGFSAF-B1; A1-RPPGFSAF-B2; A1-RPPGFSAF-B3; A1-RPPGFSAF-B4; A1-RPPGFSAF-B5; A1-RPPGFSAF-B6; A1-RPPGFSAF-B7; A1-RPPGFSAF-B8; A1-RPPGFSAF-B9; A1-RPPGFSAF-B10; A2-RPPGFSAF-B1; A2-RPPGFSAF-B2; A2-RPPGFSAF-B3; A2-RPPGFSAF-B4; A2-RPPGFSAF-B5; A1-RPPGFSAF-B6; A1-RPPGFSAF-B7; A1-RPPGFSAF-B8; A1-RPPGFSAF-B9; A1-RPPGFSAF-B10; A3-RPPGFSAF-B1; A3-RPPGFSAF-B2; A3-RPPGFSAF-B2; A3-RPPGFSAF-B3; A3-RPPGFSAF-B4; A3-RPPGFSAF-B5; A1-RPPGFSAF-B6; A1-RPPGFSAF-B7; A1-RPPGFSAF-B8; A1-RPPGFSAF-B9; A1-RPPGFSAF-B10; A4-RPPGFSAF-B1; A4-RPPGFSAF-B2; A4-RPPGFSAF-B2; A4-RPPGFSAF-B3; A4-RPPGFSAF-B4; A4-RPPGFSAF-B5; A5-RPPGFSAF-B1; A5-RPPGFSAF-B2; A5-RPPGFSAF-B2; A5-RPPGFSAF-B3; A5-RPPGFSAF-B4; A5-RPPGFSAF-B5; A1-RPPGFSAF-B6; A1-RPPGFSAF-B7; A1-RPPGFSAF-B8; A1-RPPGFSAF-B9; A1-RPPGFSAF-B10.

A1-GRGRGG-B1; A1-GRGRGG-B2; A1-GRGRGG-B3; A1-GRGRGG-B4; A1-GRGRGG-B5; A1-GRGRGG-B6; A1-GRGRGG-B7; A1-GRGRGG-B8; A1-GRGRGG-B9; A1-GRGRGG-B10; A2-GRGRGG-B1; A2-GRGRGG-B2; A2-GRGRGG-B3; A2-GRGRGG-B4; A2-GRGRGG-B5; A1-GRGRGG-B6; A1-GRGRGG-B7; A1-GRGRGG-B8; A1-GRGRGG-B9; A1-GRGRGG-B10; A3-GRGRGG-B1; A3-GRGRGG-B2; A3-GRGRGG-B2; A3-GRGRGG-B3; A3-GRGRGG-B4; A3-GRGRGG-B5; A1-GRGRGG-B6; A1-GRGRGG-B7; A1-GRGRGG-B8; A1-GRGRGG-B9; A1-GRGRGG-B10; A4-GRGRGG-B1; A4-GRGRGG-B2; A4-GRGRGG-B2; A4-GRGRGG-B3; A4-GRGRGG-B4; A4-GRGRGG-B5; A5-GRGRGG-B1; A5-GRGRGG-B2; A5-GRGRGG-B2; A5-GRGRGG-B3; A5-GRGRGG-B4; A5-GRGRGG-B5; A1-GRGRGG-B6; A1-GRGRGG-B7; A1-GRGRGG-B8; A1-GRGRGG-B9; A1-GRGRGG-B10.

A1-FNPRTFGS-B1; A1-FNPRTFGS-B2; A1-FNPRTFGS-B3; A1-FNPRTFGS-B4; A1-FNPRTFGS-B5; A1-FNPRTFGS-B6; A1-FNPRTFGS-B7; A1-FNPRTFGS-B8; A1-FNPRTFGS-B9; A1-FNPRTFGS-B10; A2-FNPRTFGS-B1; A2-FNPRTFGS-B2; A2-FNPRTFGS-B3; A2-FNPRTFGS-B4; A2-FNPRTFGS-B5; A1-FNPRTFGS-B6; A1-FNPRTFGS-B7; A1-FNPRTFGS-B8; A1-FNPRTFGS-B9; A1-FNPRTFGS-B10; A3-FNPRTFGS-B1; A3-FNPRTFGS-B2; A3-FNPRTFGS-B2; A3-FNPRTFGS-B3; A3-FNPRTFGS-B4; A3-FNPRTFGS-B5; A1-FNPRTFGS-B6; A1-FNPRTFGS-B7; A1-FNPRTFGS-B8; A1-FNPRTFGS-B9; A1-FNPRTFGS-B10; A4-FNPRTFGS-B1; A4-FNPRTFGS-B2; A4-FNPRTFGS-B2; A4-FNPRTFGS-B3; A4-FNPRTFGS-B4; A4-FNPRTFGS-B5; A5-FNPRTFGS-B1; A5-FNPRTFGS-B2; A5-FNPRTFGS-B2; A5-FNPRTFGS-B3; A5-FNPRTFGS-B4; A5-FNPRTFGS-B5; A1-FNPRTFGS-B6; A1-FNPRTFGS-B7; A1-FNPRTFGS-B8; A1-FNPRTFGS-B9; A1-FNPRTFGS-B10.

A1-RQKRFVLS-B1; A1-RQKRFVLS-B2; A1-RQKRFVLS-B3; A1-RQKRFVLS-B4; A1-RQKRFVLS-B5; A1-RQKRFVLS-B6; A1-RQKRFVLS-B7; A1-RQKRFVLS-B8; A1-RQKRFVLS-B9; A1-RQKRFVLS-B10; A2-RQKRFVLS-B1; A2-RQKRFVLS-B2; A2-RQKRFVLS-B3; A2-RQKRFVLS-B4; A2-RQKRFVLS-B5; A1-RQKRFVLS-B6; A1-RQKRFVLS-B7; A1-RQKRFVLS-B8; A1-RQKRFVLS-B9; A1-RQKRFVLS-B10; A3-RQKRFVLS-B1; A3-RQKRFVLS-B2; A3-RQKRFVLS-B2; A3-RQKRFVLS-B3; A3-RQKRFVLS-B4; A3-RQKRFVLS-B5; A1-RQKRFVLS-B6; A1-RQKRFVLS-B7; A1-RQKRFVLS-B8; A1-RQKRFVLS-B9; A1-RQKRFVLS-B10; A4-RQKRFVLS-B1; A4-RQKRFVLS-B2; A4-RQKRFVLS-B2; A4-RQKRFVLS-B3; A4-RQKRFVLS-B4; A4-RQKRFVLS-B5; A5-RQKRFVLS-B1; A5-RQKRFVLS-B2; A5-RQKRFVLS-B2; A5-RQKRFVLS-B3; A5-RQKRFVLS-B4; A5-RQKRFVLS-B5; A1-RQKRFVLS-B6; A1-RQKRFVLS-B7; A1-RQKRFVLS-B8; A1-RQKRFVLS-B9; A1-RQKRFVLS-B10.

A1-RKRRSVNP-B1; A1-RKRRSVNP-B2; A1-RKRRSVNP-B3; A1-RKRRSVNP-B4; A1-RKRRSVNP-B5; A1-RKRRSVNP-B6; A1-RKRRSVNP-B7; A1-RKRRSVNP-B8; A1-RKRRSVNP-B9; A1-RKRRSVNP-B10; A2-RKRRSVNP-B1; A2-RKRRSVNP-B2; A2-RKRRSVNP-B3; A2-RKRRSVNP-B4; A2-RKRRSVNP-B5; A1-RKRRSVNP-B6; A1-RKRRSVNP-B7; A1-RKRRSVNP-B8; A1-RKRRSVNP-B9; A1-RKRRSVNP-B10; A3-RKRRSVNP-B1; A3-RKRRSVNP-B2; A3-RKRRSVNP-B2; A3-RKRRSVNP-B3; A3-RKRRSVNP-B4; A3-RKRRSVNP-B5; A1-RKRRSVNP-B6; A1-RKRRSVNP-B7; A1-RKRRSVNP-B8; A1-RKRRSVNP-B9; A1-RKRRSVNP-B10; A4-RKRRSVNP-B1; A4-RKRRSVNP-B2; A4-RKRRSVNP-B2; A4-RKRRSVNP-B3; A4-RKRRSVNP-B4; A4-RKRRSVNP-B5; A5-RKRRSVNP-B1; A5-RKRRSVNP-B2; A5-RKRRSVNP-B2; A5-RKRRSVNP-B3; A5-RKRRSVNP-B4; A5-RKRRSVNP-B5; A1-RKRRSVNP-B6; A1-RKRRSVNP-B7; A1-RKRRSVNP-B8; A1-RKRRSVNP-B9; A1-RKRRSVNP-B10.

A1-YGPKKKRK-B1; A1-YGPKKKRK-B2; A1-YGPKKKRK-B3; A1-YGPKKKRK-B4; A1-YGPKKKRK-B5; A1-YGPKKKRK-B6; A1-YGPKKKRK-B7; A1-YGPKKKRK-B8; A1-YGPKKKRK-B9; A1-YGPKKKRK-B10; A2-YGPKKKRK-B1; A2-YGPKKKRK-B2; A2-YGPKKKRK-B3; A2-YGPKKKRK-B4; A2-YGPKKKRK-B5; A1-YGPKKKRK-B6; A1-YGPKKKRK-B7; A1-YGPKKKRK-B8; A1-YGPKKKRK-B9; A1-YGPKKKRK-B10; A3-YGPKKKRK-B1; A3-YGPKKKRK-B2; A3-YGPKKKRK-B2; A3-YGPKKKRK-B3; A3-YGPKKKRK-B4; A3-YGPKKKRK-B5; A1-YGPKKKRK-B6; A1-YGPKKKRK-B7; A1-YGPKKKRK-B8; A1-YGPKKKRK-B9; A1-YGPKKKRK-B10; A4-YGPKKKRK-B1; A4-YGPKKKRK-B2; A4-YGPKKKRK-B2; A4-YGPKKKRK-B3; A4-YGPKKKRK-B4; A4-YGPKKKRK-B5; A5-YGPKKKRK-B1; A5-YGPKKKRK-B2; A5-YGPKKKRK-B2; A5-YGPKKKRK-B3; A5-YGPKKKRK-B4; A5-YGPKKKRK-B5; A1-YGPKKKRK-B6; A1-YGPKKKRK-B7; A1-YGPKKKRK-B8; A1-YGPKKKRK-B9; A1-YGPKKKRK-B10.

A1-DDMEE-Abu-[COO]-AS-B1; A1-DDMEE-Abu-[COO]-AS-B2; A1-DDMEE-Abu-[COO]-AS-B3; A1-DDMEE-Abu-[COO]-AS-B4; A1-DDMEE-Abu-[COO]-AS-B5; A1-DDMEE-Abu-[COO]-AS-B6; A1-DDMEE-Abu-[COO]-AS-B7; A1-DDMEE-Abu-[COO]-AS-B8; A1-DDMEE-Abu-[COO]-AS-B9; A1-DDMEE-Abu-[COO]-AS-B10; A2-DDMEE-Abu-[COO]-AS-B1; A2-DDMEE-Abu-[COO]-AS-B2; A2-DDMEE-Abu-[COO]-AS-B3; A2-DDMEE-Abu-[COO]-AS-B4; A2-DDMEE-Abu-[COO]-AS-B5; A1-DDMEE-Abu-[COO]-AS-B6; A1-DDMEE-Abu-[COO]-AS-B7; A1-DDMEE-Abu-[COO]-AS-B8; A1-DDMEE-Abu-[COO]-AS-B9; A1-DDMEE-Abu-[COO]-AS-B10; A3-DDMEE-Abu-[COO]-AS-B1; A3-DDMEE-Abu-[COO]-AS-B2; A3-DDMEE-Abu-[COO]-AS-B2; A3-DDMEE-Abu-[COO]-AS-B3; A3-DDMEE-Abu-[COO]-AS-B4; A3-DDMEE-Abu-[COO]-AS-B5; A1-DDMEE-Abu-[COO]-AS-B6; A1-DDMEE-Abu-[COO]-AS-B7; A1-DDMEE-Abu-[COO]-AS-B8; A1-DDMEE-Abu-[COO]-AS-B9; A1-DDMEE-Abu-[COO]-AS-B10; A4-DDMEE-Abu-[COO]-AS-B1; A4-DDMEE-Abu-[COO]-AS-B2; A4-DDMEE-Abu-[COO]-AS-B2; A4-DDMEE-Abu-[COO]-AS-B3; A4-DDMEE-Abu-[COO]-AS-B4; A4-DDMEE-Abu-[COO]-AS-B5; A5-DDMEE-Abu-[COO]-AS-B1; A5-DDMEE-Abu-[COO]-AS-B2; A5-DDMEE-Abu-[COO]-AS-B2; A5-DDMEE-Abu-[COO]-AS-B3; A5-DDMEE-Abu-[COO]-AS-B4; A5-DDMEE-Abu-[COO]-AS-B5; A1-DDMEE-Abu-[COO]-AS-B6; A1-DDMEE-Abu-[COO]-AS-B7; A1-DDMEE-Abu-[COO]-AS-B8; A1-DDMEE-Abu-[COO]-AS-B9; A1-DDMEE-Abu-[COO]-AS-B10.

A1-EDVVACSMSY-B1; A1-EDVVACSMSY-B2; A1-EDVVACSMSY-B3; A1-EDVVACSMSY-B4; A1-EDVVACSMSY-B5; A1-EDVVACSMSY-B6; A1-EDVVACSMSY-B7; A1-EDVVACSMSY-B8; A1-EDVVACSMSY-B9; A1-EDVVACSMSY-B10; A2-EDVVACSMSY-B1; A2-EDVVACSMSY-B2; A2-EDVVACSMSY-B3; A2-EDVVACSMSY-B4; A2-EDVVACSMSY-B5; A1-EDVVACSMSY-B6; A1-EDVVACSMSY-B7; A1-EDVVACSMSY-B8; A1-EDVVACSMSY-B9; A1-EDVVACSMSY-B10; A3-EDVVACSMSY-B1; A3-EDVVACSMSY-B2; A3-EDVVACSMSY-B2; A3-EDVVACSMSY-B3; A3-EDVVACSMSY-B4; A3-EDVVACSMSY-B5; A1-EDVVACSMSY-B6; A1-EDVVACSMSY-B7; A1-EDVVACSMSY-B8; A1-EDVVACSMSY-B9; A1-EDVVACSMSY-B10; A4-EDVVACSMSY-B1; A4-EDVVACSMSY-B2; A4-EDVVACSMSY-B2; A4-EDVVACSMSY-B3; A4-EDVVACSMSY-B4; A4-EDVVACSMSY-B5; A5-EDVVACSMSY-B1; A5-EDVVACSMSY-B2; A5-EDVVACSMSY-B2; A5-EDVVACSMSY-B3; A5-EDVVACSMSY-B4; A5-EDVVACSMSY-B5; A1-EDVVACSMSY-B6; A1-EDVVACSMSY-B7; A1-EDVVACSMSY-B8; A1-EDVVACSMSY-B9; A1-EDVVACSMSY-B10.

A1-VEDVVCCSM-B1; A1-VEDVVCCSM-B2; A1-VEDVVCCSM-B3; A1-VEDVVCCSM-B4; A1-VEDVVCCSM-B5; A1-VEDVVCCSM-B6; A1-VEDVVCCSM-B7; A1-VEDVVCCSM-B8; A1-VEDVVCCSM-B9; A1-VEDVVCCSM-B10; A2-VEDVVCCSM-B1; A2-VEDVVCCSM-B2; A2-VEDVVCCSM-B3; A2-VEDVVCCSM-B4; A2-VEDVVCCSM-B5; A1-VEDVVCCSM-B6; A1-VEDVVCCSM-B7; A1-VEDVVCCSM-B8; A1-VEDVVCCSM-B9; A1-VEDVVCCSM-B10; A3-VEDVVCCSM-B1; A3-VEDVVCCSM-B2; A3-VEDVVCCSM-B2; A3-VEDVVCCSM-B3; A3-VEDVVCCSM-B4; A3-VEDVVCCSM-B5; A1-VEDVVCCSM-B6; A1-VEDVVCCSM-B7; A1-VEDVVCCSM-B8; A1-VEDVVCCSM-B9; A1-VEDVVCCSM-B10; A4-VEDVVCCSM-B1; A4-VEDVVCCSM-B2; A4-VEDVVCCSM-B2; A4-VEDVVCCSM-B3; A4-VEDVVCCSM-B4; A4-VEDVVCCSM-B5; A5-VEDVVCCSM-B1; A5-VEDVVCCSM-B2; A5-VEDVVCCSM-B2; A5-VEDVVCCSM-B3; A5-VEDVVCCSM-B4; A5-VEDVVCCSM-B5; A1-VEDVVCCSM-B6; A1-VEDVVCCSM-B7; A1-VEDVVCCSM-B8; A1-VEDVVCCSM-B9; A1-VEDVVCCSM-B10.

A1-SQNYPIVQ-B1; A1-SQNYPIVQ-B2; A1-SQNYPIVQ-B3; A1-SQNYPIVQ-B4; A1-SQNYPIVQ-B5; A1-SQNYPIVQ-B6; A1-SQNYPIVQ-B7; A1-SQNYPIVQ-B8; A1-SQNYPIVQ-B9; A1-SQNYPIVQ-B10; A2-SQNYPIVQ-B1; A2-SQNYPIVQ-B2; A2-SQNYPIVQ-B3; A2-SQNYPIVQ-B4; A2-SQNYPIVQ-B5; A1-SQNYPIVQ-B6; A1-SQNYPIVQ-B7; A1-SQNYPIVQ-B8; A1-SQNYPIVQ-B9; A1-SQNYPIVQ-B10; A3-SQNYPIVQ-B1; A3-SQNYPIVQ-B2; A3-SQNYPIVQ-B2; A3-SQNYPIVQ-B3; A3-SQNYPIVQ-B4; A3-SQNYPIVQ-B5; A1-SQNYPIVQ-B6; A1-SQNYPIVQ-B7; A1-SQNYPIVQ-B8; A1-SQNYPIVQ-B9; A1-SQNYPIVQ-B10; A4-SQNYPIVQ-B1; A4-SQNYPIVQ-B2; A4-SQNYPIVQ-B2; A4-SQNYPIVQ-B3; A4-SQNYPIVQ-B4; A4-SQNYPIVQ-B5; A5-SQNYPIVQ-B1; A5-SQNYPIVQ-B2; A5-SQNYPIVQ-B2; A5-SQNYPIVQ-B3; A5-SQNYPIVQ-B4; A5-SQNYPIVQ-B5; A1-SQNYPIVQ-B6; A1-SQNYPIVQ-B7; A1-SQNYPIVQ-B8; A1-SQNYPIVQ-B9; A1-SQNYPIVQ-B10.

A1-PPEELKFQ-B1; A1-PPEELKFQ-B2; A1-PPEELKFQ-B3; A1-PPEELKFQ-B4; A1-PPEELKFQ-B5; A1-PPEELKFQ-B6; A1-PPEELKFQ-B7; A1-PPEELKFQ-B8; A1-PPEELKFQ-B9; A1-PPEELKFQ-B10; A2-PPEELKFQ-B1; A2-PPEELKFQ-B2; A2-PPEELKFQ-B3; A2-PPEELKFQ-B4; A2-PPEELKFQ-B5; A1-PPEELKFQ-B6; A1-PPEELKFQ-B7; A1-PPEELKFQ-B8; A1-PPEELKFQ-B9; A1-PPEELKFQ-B10; A3-PPEELKFQ-B1; A3-PPEELKFQ-B2; A3-PPEELKFQ-B2; A3-PPEELKFQ-B3; A3-PPEELKFQ-B4; A3-PPEELKFQ-B5; A1-PPEELKFQ-B6; A1-PPEELKFQ-B7; A1-PPEELKFQ-B8; A1-PPEELKFQ-B9; A1-PPEELKFQ-B10; A4-PPEELKFQ-B1; A4-PPEELKFQ-B2; A4-PPEELKFQ-B2; A4-PPEELKFQ-B3; A4-PPEELKFQ-B4; A4-PPEELKFQ-B5; A5-PPEELKFQ-B1; A5-PPEELKFQ-B2; A5-PPEELKFQ-B2; A5-PPEELKFQ-B3; A5-PPEELKFQ-B4; A5-PPEELKFQ-B5; A1-PPEELKFQ-B6; A1-PPEELKFQ-B7; A1-PPEELKFQ-B8; A1-PPEELKFQ-B9; A1-PPEELKFQ-B10.

A1-AIFQGPID-B1; A1-AIFQGPID-B2; A1-AIFQGPID-B3; A1-AIFQGPID-B4; A1-AIFQGPID-B5; A1-AIFQGPID-B6; A1-AIFQGPID-B7; A1-AIFQGPID-B8; A1-AIFQGPID-B9; A1-AIFQGPID-B10; A2-AIFQGPID-B1; A2-AIFQGPID-B2; A2-AIFQGPID-B3; A2-AIFQGPID-B4; A2-AIFQGPID-B5; A1-AIFQGPID-B6; A1-AIFQGPID-B7; A1-AIFQGPID-B8; A1-AIFQGPID-B9; A1-AIFQGPID-B10; A3-AIFQGPID-B1; A3-AIFQGPID-B2; A3-AIFQGPID-B2; A3-AIFQGPID-B3; A3-AIFQGPID-B4; A3-AIFQGPID-B5; A1-AIFQGPID-B6; A1-AIFQGPID-B7; A1-AIFQGPID-B8; A1-AIFQGPID-B9; A1-AIFQGPID-B10; A4-AIFQGPID-B1; A4-AIFQGPID-B2; A4-AIFQGPID-B2; A4-AIFQGPID-B3; A4-AIFQGPID-B4; A4-AIFQGPID-B5; A5-AIFQGPID-B1; A5-AIFQGPID-B2; A5-AIFQGPID-B2; A5-AIFQGPID-B3; A5-AIFQGPID-B4; A5-AIFQGPID-B5; A1-AIFQGPID-B6; A1-AIFQGPID-B7; A1-AIFQGPID-B8; A1-AIFQGPID-B9; A1-AIFQGPID-B10.

A1-GFSPFRASRV-B1; A1-GFSPFRASRV-B2; A1-GFSPFRASRV-B3; A1-GFSPFRASRV-B4; A1-GFSPFRASRV-B5; A1-GFSPFRASRV-B6; A1-GFSPFRASRV-B7; A1-GFSPFRASRV-B8; A1-GFSPFRASRV-B9; A1-GFSPFRASRV-B10; A2-GFSPFRASRV-B1; A2-GFSPFRASRV-B2; A2-GFSPFRASRV-B3; A2-GFSPFRASRV-B4; A2-GFSPFRASRV-B5; A1-GFSPFRASRV-B6; A1-GFSPFRASRV-B7; A1-GFSPFRASRV-B8; A1-GFSPFRASRV-B9; A1-GFSPFRASRV-B10; A3-GFSPFRASRV-B1; A3-GFSPFRASRV-B2; A3-GFSPFRASRV-B2; A3-GFSPFRASRV-B3; A3-GFSPFRASRV-B4; A3-GFSPFRASRV-B5; A1-GFSPFRASRV-B6; A1-GFSPFRASRV-B7; A1-GFSPFRASRV-B8; A1-GFSPFRASRV-B9; A1-GFSPFRASRV-B10; A4-GFSPFRASRV-B1; A4-GFSPFRASRV-B2; A4-GFSPFRASRV-B2; A4-GFSPFRASRV-B3; A4-GFSPFRASRV-B4; A4-GFSPFRASRV-B5; A5-GFSPFRASRV-B1; A5-GFSPFRASRV-B2; A5-GFSPFRASRV-B2; A5-GFSPFRASRV-B3; A5-GFSPFRASRV-B4; A5-GFSPFRASRV-B5; A1-GFSPFRASRV-B6; A1-GFSPFRASRV-B7; A1-GFSPFRASRV-B8; A1-GFSPFRASRV-B9; A1-GFSPFRASRV-B10.

A1-GKAFRR-B1; A1-GKAFRR-B2; A1-GKAFRR-B3; A1-GKAFRR-B4; A1-GKAFRR-B5; A1-GKAFRR-B6; A1-GKAFRR-B7; A1-GKAFRR-B8; A1-GKAFRR-B9; A1-GKAFRR-B10; A2-GKAFRR-B1; A2-GKAFRR-B2; A2-GKAFRR-B3; A2-GKAFRR-B4; A2-GKAFRR-B5; A1-GKAFRR-B6; A1-GKAFRR-B7; A1-GKAFRR-B8; A1-GKAFRR-B9; A1-GKAFRR-B10; A3-GKAFRR-B1; A3-GKAFRR-B2; A3-GKAFRR-B2; A3-GKAFRR-B3; A3-GKAFRR-B4; A3-GKAFRR-B5; A1-GKAFRR-B6; A1-GKAFRR-B7; A1-GKAFRR-B8; A1-GKAFRR-B9; A1-GKAFRR-B10; A4-GKAFRR-B1; A4-GKAFRR-B2; A4-GKAFRR-B2; A4-GKAFRR-B3; A4-GKAFRR-B4; A4-GKAFRR-B5; A5-GKAFRR-B1; A5-GKAFRR-B2; A5-GKAFRR-B2; A5-GKAFRR-B3; A5-GKAFRR-B4; A5-GKAFRR-B5; A1-GKAFRR-B6; A1-GKAFRR-B7; A1-GKAFRR-B8; A1-GKAFRR-B9; A1-GKAFRR-B10.

A1-AFRFSQ-B1; A1-AFRFSQ-B2; A1-AFRFSQ-B3; A1-AFRFSQ-B4; A1-AFRFSQ-B5; A1-AFRFSQ-B6; A1-AFRFSQ-B7; A1-AFRFSQ-B8; A1-AFRFSQ-B9; A1-AFRFSQ-B10; A2-AFRFSQ-B1; A2-AFRFSQ-B2; A2-AFRFSQ-B3; A2-AFRFSQ-B4; A2-AFRFSQ-B5; A1-AFRFSQ-B6; A1-AFRFSQ-B7; A1-AFRFSQ-B8; A1-AFRFSQ-B9; A1-AFRFSQ-B10; A3-AFRFSQ-B1; A3-AFRFSQ-B2; A3-AFRFSQ-B2; A3-AFRFSQ-B3; A3-AFRFSQ-B4; A3-AFRFSQ-B5; A1-AFRFSQ-B6; A1-AFRFSQ-B7; A1-AFRFSQ-B8; A1-AFRFSQ-B9; A1-AFRFSQ-B10; A4-AFRFSQ-B1; A4-AFRFSQ-B2; A4-AFRFSQ-B2; A4-AFRFSQ-B3; A4-AFRFSQ-B4; A4-AFRFSQ-B5; A5-AFRFSQ-B1; A5-AFRFSQ-B2; A5-AFRFSQ-B2; A5-AFRFSQ-B3; A5-AFRFSQ-B4; A5-AFRFSQ-B5; A1-AFRFSQ-B6; A1-AFRFSQ-B7; A1-AFRFSQ-B8; A1-AFRFSQ-B9; A1-AFRFSQ-B10.

A1-GVGVTETPLM-B1; A1-GVGVTETPLM-B2; A1-GVGVTETPLM-B3; A1-GVGVTETPLM-B4; A1-GVGVTETPLM-B5; A1-GVGVTETPLM-B6; A1-GVGVTETPLM-B7; A1-GVGVTETPLM-B8; A1-GVGVTETPLM-B9; A1-GVGVTETPLM-B10; A2-GVGVTETPLM-B1; A2-GVGVTETPLM-B2; A2-GVGVTETPLM-B3; A2-GVGVTETPLM-B4; A2-GVGVTETPLM-B5; A1-GVGVTETPLM-B6; A1-GVGVTETPLM-B7; A1-GVGVTETPLM-B8; A1-GVGVTETPLM-B9; A1-GVGVTETPLM-B10; A3-GVGVTETPLM-B1; A3-GVGVTETPLM-B2; A3-GVGVTETPLM-B2; A3-GVGVTETPLM-B3; A3-GVGVTETPLM-B4; A3-GVGVTETPLM-B5; A1-GVGVTETPLM-B6; A1-GVGVTETPLM-B7; A1-GVGVTETPLM-B8; A1-GVGVTETPLM-B9; A1-GVGVTETPLM-B10; A4-GVGVTETPLM-B1; A4-GVGVTETPLM-B2; A4-GVGVTETPLM-B2; A4-GVGVTETPLM-B3; A4-GVGVTETPLM-B4; A4-GVGVTETPLM-B5; A5-GVGVTETPLM-B1; A5-GVGVTETPLM-B2; A5-GVGVTETPLM-B2; A5-GVGVTETPLM-B3; A5-GVGVTETPLM-B4; A5-GVGVTETPLM-B5; A1-GVGVTETPLM-B6; A1-GVGVTETPLM-B7; A1-GVGVTETPLM-B8; A1-GVGVTETPLM-B9; A1-GVGVTETPLM-B10.

A1-RRKKVYPYPYPMEGTIA-B1; A1-RRKKVYPYPYPMEGTIA-B2; A1-RRKKVYPYPYPMEGTIA-B3; A1-RRKKVYPYPYPMEGTIA-B4; A1-RRKKVYPYPYPMEGTIA-B5; A1-RRKKVYPYPYPMEGTIA-B6; A1-RRKKVYPYPYPMEGTIA-B7; A1-RRKKVYPYPYPMEGTIA-B8; A1-RRKKVYPYPYPMEGTIA-B9; A1-RRKKVYPYPYPMEGTIA-B10; A2-RRKKVYPYPYPMEGTIA-B1; A2-RRKKVYPYPYPMEGTIA-B2; A2-RRKKVYPYPYPMEGTIA-B3; A2-RRKKVYPYPYPMEGTIA-B4; A2-RRKKVYPYPYPMEGTIA-B5; A1-RRKKVYPYPYPMEGTIA-B6; A1-RRKKVYPYPYPMEGTIA-B7; A1-RRKKVYPYPYPMEGTIA-B8; A1-RRKKVYPYPYPMEGTIA-B9; A1-RRKKVYPYPYPMEGTIA-B10; A3-RRKKVYPYPYPMEGTIA-B1; A3-RRKKVYPYPYPMEGTIA-B2; A3-RRKKVYPYPYPMEGTIA-B2; A3-RRKKVYPYPYPMEGTIA-B3; A3-RRKKVYPYPYPMEGTIA-B4; A3-RRKKVYPYPYPMEGTIA-B5; A1-RRKKVYPYPYPMEGTIA-B6; A1-RRKKVYPYPYPMEGTIA-B7; A1-RRKKVYPYPYPMEGTIA-B8; A1-RRKKVYPYPYPMEGTIA-B9; A1-RRKKVYPYPYPMEGTIA-B10; A4-RRKKVYPYPYPMEGTIA-B1; A4-RRKKVYPYPYPMEGTIA-B2; A4-RRKKVYPYPYPMEGTIA-B2; A4-RRKKVYPYPYPMEGTIA-B3; A4-RRKKVYPYPYPMEGTIA-B4; A4-RRKKVYPYPYPMEGTIA-B5; A5-RRKKVYPYPYPMEGTIA-B1; A5-RRKKVYPYPYPMEGTIA-B2; A5-RRKKVYPYPYPMEGTIA-B2; A5-RRKKVYPYPYPMEGTIA-B3; A5-RRKKVYPYPYPMEGTIA-B4; A5-RRKKVYPYPYPMEGTIA-B5; A1-RRKKVYPYPYPMEGTIA-B6; A1-RRKKVYPYPYPMEGTIA-B7; A1-RRKKVYPYPYPMEGTIA-B8; A1-RRKKVYPYPYPMEGTIA-B9; A1-RRKKVYPYPYPMEGTIA-B10.

A1-PTLQLPLA-B1; A1-PTLQLPLA-B2; A1-PTLQLPLA-B3; A1-PTLQLPLA-B4; A1-PTLQLPLA-B5; A1-PTLQLPLA-B6; A1-PTLQLPLA-B7; A1-PTLQLPLA-B8; A1-PTLQLPLA-B9; A1-PTLQLPLA-B10; A2-PTLQLPLA-B1; A2-PTLQLPLA-B2; A2-PTLQLPLA-B3; A2-PTLQLPLA-B4; A2-PTLQLPLA-B5; A1-PTLQLPLA-B6; A1-PTLQLPLA-B7; A1-PTLQLPLA-B8; A1-PTLQLPLA-B9; A1-PTLQLPLA-B10; A3-PTLQLPLA-B1; A3-PTLQLPLA-B2; A3-PTLQLPLA-B2; A3-PTLQLPLA-B3; A3-PTLQLPLA-B4; A3-PTLQLPLA-B5; A1-PTLQLPLA-B6; A1-PTLQLPLA-B7; A1-PTLQLPLA-B8; A1-PTLQLPLA-B9; A1-PTLQLPLA-B10; A4-PTLQLPLA-B1; A4-PTLQLPLA-B2; A4-PTLQLPLA-B2; A4-PTLQLPLA-B3; A4-PTLQLPLA-B4; A4-PTLQLPLA-B5; A5-PTLQLPLA-B1; A5-PTLQLPLA-B2; A5-PTLQLPLA-B2; A5-PTLQLPLA-B3; A5-PTLQLPLA-B4; A5-PTLQLPLA-B5; A1-PTLQLPLA-B6; A1-PTLQLPLA-B7; A1-PTLQLPLA-B8; A1-PTLQLPLA-B9; A1-PTLQLPLA-B10.

A1-ER-Nle-FLSFP-B1; A1-ER-Nle-FLSFP-B2; A1-ER-Nle-FLSFP-B3; A1-ER-Nle-FLSFP-B4; A1-ER-Nle-FLSFP-B5; A1-ER-Nle-FLSFP-B6; A1-ER-Nle-FLSFP-B7; A1-ER-Nle-FLSFP-B8; A1-ER-Nle-FLSFP-B9; A1-ER-Nle-FLSFP-B10; A2-ER-Nle-FLSFP-B1; A2-ER-Nle-FLSFP-B2; A2-ER-Nle-FLSFP-B3; A2-ER-Nle-FLSFP-B4; A2-ER-Nle-FLSFP-B5; A1-ER-Nle-FLSFP-B6; A1-ER-Nle-FLSFP-B7; A1-ER-Nle-FLSFP-B8; A1-ER-Nle-FLSFP-B9; A1-ER-Nle-FLSFP-B10; A3-ER-Nle-FLSFP-B1; A3-ER-Nle-FLSFP-B2; A3-ER-Nle-FLSFP-B2; A3-ER-Nle-FLSFP-B3; A3-ER-Nle-FLSFP-B4; A3-ER-Nle-FLSFP-B5; A1-ER-Nle-FLSFP-B6; A1-ER-Nle-FLSFP-B7; A1-ER-Nle-FLSFP-B8; A1-ER-Nle-FLSFP-B9; A1-ER-Nle-FLSFP-B10; A4-ER-Nle-FLSFP-B1; A4-ER-Nle-FLSFP-B2; A4-ER-Nle-FLSFP-B2; A4-ER-Nle-FLSFP-B3; A4-ER-Nle-FLSFP-B4; A4-ER-Nle-FLSFP-B5; A5-ER-Nle-FLSFP-B1; A5-ER-Nle-FLSFP-B2; A5-ER-Nle-FLSFP-B2; A5-ER-Nle-FLSFP-B3; A5-ER-Nle-FLSFP-B4; A5-ER-Nle-FLSFP-B5; A1-ER-Nle-FLSFP-B6; A1-ER-Nle-FLSFP-B7; A1-ER-Nle-FLSFP-B8; A1-ER-Nle-FLSFP-B9; A1-ER-Nle-FLSFP-B10.

A1-ERMFLSFP-B1; A1-ERMFLSFP-B2; A1-ERMFLSFP-B3; A1-ERMFLSFP-B4; A1-ERMFLSFP-B5; A1-ERMFLSFP-B6; A1-ERMFLSFP-B7; A1-ERMFLSFP-B8; A1-ERMFLSFP-B9; A1-ERMFLSFP-B10; A2-ERMFLSFP-B1; A2-ERMFLSFP-B2; A2-ERMFLSFP-B3; A2-ERMFLSFP-B4; A2-ERMFLSFP-B5; A1-ERMFLSFP-B6; A1-ERMFLSFP-B7; A1-ERMFLSFP-B8; A1-ERMFLSFP-B9; A1-ERMFLSFP-B10; A3-ERMFLSFP-B1; A3-ERMFLSFP-B2; A3-ERMFLSFP-B2; A3-ERMFLSFP-B3; A3-ERMFLSFP-B4; A3-ERMFLSFP-B5; A1-ERMFLSFP-B6; A1-ERMFLSFP-B7; A1-ERMFLSFP-B8; A1-ERMFLSFP-B9; A1-ERMFLSFP-B10; A4-ERMFLSFP-B1; A4-ERMFLSFP-B2; A4-ERMFLSFP-B2; A4-ERMFLSFP-B3; A4-ERMFLSFP-B4; A4-ERMFLSFP-B5; A5-ERMFLSFP-B1; A5-ERMFLSFP-B2; A5-ERMFLSFP-B2; A5-ERMFLSFP-B3; A5-ERMFLSFP-B4; A5-ERMFLSFP-B5; A1-ERMFLSFP-B6; A1-ERMFLSFP-B7; A1-ERMFLSFP-B8; A1-ERMFLSFP-B9; A1-ERMFLSFP-B10.

A1-ALERMFLSFP-B1; A1-ALERMFLSFP-B2; A1-ALERMFLSFP-B3; A1-ALERMFLSFP-B4; A1-ALERMFLSFP-B5; A1-ALERMFLSFP-B6; A1-ALERMFLSFP-B7; A1-ALERMFLSFP-B8; A1-ALERMFLSFP-B9; A1-ALERMFLSFP-B10; A2-ALERMFLSFP-B1; A2-ALERMFLSFP-B2; A2-ALERMFLSFP-B3; A2-ALERMFLSFP-B4; A2-ALERMFLSFP-B5; A1-ALERMFLSFP-B6; A1-ALERMFLSFP-B7; A1-ALERMFLSFP-B8; A1-ALERMFLSFP-B9; A1-ALERMFLSFP-B10; A3-ALERMFLSFP-B1; A3-ALERMFLSFP-B2; A3-ALERMFLSFP-B2; A3-ALERMFLSFP-B3; A3-ALERMFLSFP-B4; A3-ALERMFLSFP-B5; A1-ALERMFLSFP-B6; A1-ALERMFLSFP-B7; A1-ALERMFLSFP-B8; A1-ALERMFLSFP-B9; A1-ALERMFLSFP-B10; A4-ALERMFLSFP-B1; A4-ALERMFLSFP-B2; A4-ALERMFLSFP-B2; A4-ALERMFLSFP-B3; A4-ALERMFLSFP-B4; A4-ALERMFLSFP-B5; A5-ALERMFLSFP-B1; A5-ALERMFLSFP-B2; A5-ALERMFLSFP-B2; A5-ALERMFLSFP-B3; A5-ALERMFLSFP-B4; A5-ALERMFLSFP-B5; A1-ALERMFLSFP-B6; A1-ALERMFLSFP-B7; A1-ALERMFLSFP-B8; A1-ALERMFLSFP-B9; A1-ALERMFLSFP-B10.

A1-KGP-Cha-Abu-Smc-HA-B1; A1-KGPCha-Abu-Smc-HA-B2; A1-KGP-Cha-Abu-Smc-HA-B3; A1-KGP-Cha-Abu-Smc-HA-B4; A1-KGP-Cha-Abu-Smc-HA-B5; A1-KGP-Cha-Abu-Smc-HA-B6; A1-KGP-Cha-Abu-Smc-HA-B7; A1-KGP-Cha-Abu-Smc-HA-B8; A1-KGP-Cha-Abu-Smc-HA-B9; A1-KGP-Cha-Abu-Smc-HA-B10; A2-KGP-Cha-Abu-Smc-HA-B1; A2-KGP-Cha-Abu-Smc-HA-B2; A2-KGP-Cha-Abu-Smc-HA-B3; A2-KGP-Cha-Abu-Smc-HA-B4; A2-KGP-Cha-Abu-Smc-HA-B5; A1-KGP-Cha-Abu-Smc-HA-B6; A1-KGP-Cha-Abu-Smc-HA-B7; A1-KGP-Cha-Abu-Smc-HA-B8; A1-KGP-Cha-Abu-Smc-HA-B9; A1-KGP-Cha-Abu-Smc-HA-B10; A3-KGP-Cha-Abu-Smc-HA-B1; A3-KGP-Cha-Abu-Smc-HA-B2; A3-KGP-Cha-Abu-Smc-HA-B2; A3-KGP-Cha-Abu-Smc-HA-B3; A3-KGP-Cha-Abu-Smc-HA-B4; A3-KGP-Cha-Abu-Smc-HA-B5; A1-KGP-Cha-Abu-Smc-HA-B6; A1-KGP-Cha-Abu-Smc-HA-B7; A1-KGP-Cha-Abu-Smc-HA-B8; A1-KGP-Cha-Abu-Smc-HA-B9; A1-KGP-Cha-Abu-Smc-HA-B10; A4-KGP-Cha-Abu-Smc-HA-B1; A4-KGP-Cha-Abu-Smc-HA-B2; A4-KGP-Cha-Abu-Smc-HA-B2; A4-KGP-Cha-Abu-Smc-HA-B3; A4-KGP-Cha-Abu-Smc-HA-B4; A4-KGP-Cha-Abu-Smc-HA-B5; A5-KGP-Cha-Abu-Smc-HA-B1; A5-KGP-Cha-Abu-Smc-HA-B2; A5-KGP-Cha-Abu-Smc-HA-B2; A5-KGP-Cha-Abu-Smc-HA-B3; A5-KGP-Cha-Abu-Smc-HA-B4; A5-KGP-Cha-Abu-Smc-HA-B5; A1-KGP-Cha-Abu-Smc-HA-B6; A1-KGP-Cha-Abu-Smc-HA-B7; A1-KGP-Cha-Abu-Smc-HA-B8; A1-KGP-Cha-Abu-Smc-HA-B9; A1-KGP-Cha-Abu-Smc-HA-B10.

A1-GSHLVEAL-B1; A1-GSHLVEAL-B2; A1-GSHLVEAL-B3; A1-GSHLVEAL-B4; A1-GSHLVEAL-B5; A1-GSHLVEAL-B6; A1-GSHLVEAL-B7; A1-GSHLVEAL-B8; A1-GSHLVEAL-B9; A1-GSHLVEAL-B10; A2-GSHLVEAL-B1; A2-GSHLVEAL-B2; A2-GSHLVEAL-B3; A2-GSHLVEAL-B4; A2-GSHLVEAL-B5; A1-GSHLVEAL-B6; A1-GSHLVEAL-B7; A1-GSHLVEAL-B8; A1-GSHLVEAL-B9; A1-GSHLVEAL-B10; A3-GSHLVEAL-B1; A3-GSHLVEAL-B2; A3-GSHLVEAL-B2; A3-GSHLVEAL-B3; A3-GSHLVEAL-B4; A3-GSHLVEAL-B5; A1-GSHLVEAL-B6; A1-GSHLVEAL-B7; A1-GSHLVEAL-B8; A1-GSHLVEAL-B9; A1-GSHLVEAL-B10; A4-GSHLVEAL-B1; A4-GSHLVEAL-B2; A4-GSHLVEAL-B2; A4-GSHLVEAL-B3; A4-GSHLVEAL-B4; A4-GSHLVEAL-B5; A5-GSHLVEAL-B1; A5-GSHLVEAL-B2; A5-GSHLVEAL-B2; A5-GSHLVEAL-B3; A5-GSHLVEAL-B4; A5-GSHLVEAL-B5; A1-GSHLVEAL-B6; A1-GSHLVEAL-B7; A1-GSHLVEAL-B8; A1-GSHLVEAL-B9; A1-GSHLVEAL-B10.

A1-PKAKSHAP-B1; A1-PKAKSHAP-B2; A1-PKAKSHAP-B3; A1-PKAKSHAP-B4; A1-PKAKSHAP-B5; A1-PKAKSHAP-B6; A1-PKAKSHAP-B7; A1-PKAKSHAP-B8; A1-PKAKSHAP-B9; A1-PKAKSHAP-B10; A2-PKAKSHAP-B1; A2-PKAKSHAP-B2; A2-PKAKSHAP-B3; A2-PKAKSHAP-B4; A2-PKAKSHAP-B5; A1-PKAKSHAP-B6; A1-PKAKSHAP-B7; A1-PKAKSHAP-B8; A1-PKAKSHAP-B9; A1-PKAKSHAP-B10; A3-PKAKSHAP-B1; A3-PKAKSHAP-B2; A3-PKAKSHAP-B2; A3-PKAKSHAP-B3; A3-PKAKSHAP-B4; A3-PKAKSHAP-B5; A1-PKAKSHAP-B6; A1-PKAKSHAP-B7; A1-PKAKSHAP-B8; A1-PKAKSHAP-B9; A1-PKAKSHAP-B10; A4-PKAKSHAP-B1; A4-PKAKSHAP-B2; A4-PKAKSHAP-B2; A4-PKAKSHAP-B3; A4-PKAKSHAP-B4; A4-PKAKSHAP-B5; A5-PKAKSHAP-B1; A5-PKAKSHAP-B2; A5-PKAKSHAP-B2; A5-PKAKSHAP-B3; A5-PKAKSHAP-B4; A5-PKAKSHAP-B5; A1-PKAKSHAP-B6; A1-PKAKSHAP-B7; A1-PKAKSHAP-B8; A1-PKAKSHAP-B9; A1-PKAKSHAP-B10.

A1-PQFRIKG-B1; A1-PQFRIKG-B2; A1-PQFRIKG-B3; A1-PQFRIKG-B4; A1-PQFRIKG-B5; A1-PQFRIKG-B6; A1-PQFRIKG-B7; A1-PQFRIKG-B8; A1-PQFRIKG-B9; A1-PQFRIKG-B10; A2-PQFRIKG-B1; A2-PQFRIKG-B2; A2-PQFRIKG-B3; A2-PQFRIKG-B4; A2-PQFRIKG-B5; A1-PQFRIKG-B6; A1-PQFRIKG-B7; A1-PQFRIKG-B8; A1-PQFRIKG-B9; A1-PQFRIKG-B10; A3-PQFRIKG-B1; A3-PQFRIKG-B2; A3-PQFRIKG-B2; A3-PQFRIKG-B3; A3-PQFRIKG-B4; A3-PQFRIKG-B5; A1-PQFRIKG-B6; A1-PQFRIKG-B7; A1-PQFRIKG-B8; A1-PQFRIKG-B9; A1-PQFRIKG-B10; A4-PQFRIKG-B1; A4-PQFRIKG-B2; A4-PQFRIKG-B2; A4-PQFRIKG-B3; A4-PQFRIKG-B4; A4-PQFRIKG-B5; A5-PQFRIKG-B1; A5-PQFRIKG-B2; A5-PQFRIKG-B2; A5-PQFRIKG-B3; A5-PQFRIKG-B4; A5-PQFRIKG-B5; A1-PQFRIKG-B6; A1-PQFRIKG-B7; A1-PQFRIKG-B8; A1-PQFRIKG-B9; A1-PQFRIKG-B10.

A1-ALERMFLSFP-B1; A1-ALERMFLSFP-B2; A1-ALERMFLSFP-B3; A1-ALERMFLSFP-B4; A1-ALERMFLSFP-B5; A1-ALERMFLSFP-B6; A1-ALERMFLSFP-B7; A1-ALERMFLSFP-B8; A1-ALERMFLSFP-B9; A1-ALERMFLSFP-B10; A2-ALERMFLSFP-B1; A2-ALERMFLSFP-B2; A2-ALERMFLSFP-B3; A2-ALERMFLSFP-B4; A2-ALERMFLSFP-B5; A1-ALERMFLSFP-B6; A1-ALERMFLSFP-B7; A1-ALERMFLSFP-B8; A1-ALERMFLSFP-B9; A1-ALERMFLSFP-B10; A3-ALERMFLSFP-B1; A3-ALERMFLSFP-B2; A3-ALERMFLSFP-B2; A3-ALERMFLSFP-B3; A3-ALERMFLSFP-B4; A3-ALERMFLSFP-B5; A1-ALERMFLSFP-B6; A1-ALERMFLSFP-B7; A1-ALERMFLSFP-B8; A1-ALERMFLSFP-B9; A1-ALERMFLSFP-B10; A4-ALERMFLSFP-B1; A4-ALERMFLSFP-B2; A4-ALERMFLSFP-B2; A4-ALERMFLSFP-B3; A4-ALERMFLSFP-B4; A4-ALERMFLSFP-B5; A5-ALERMFLSFP-B1; A5-ALERMFLSFP-B2; A5-ALERMFLSFP-B2; A5-ALERMFLSFP-B3; A5-ALERMFLSFP-B4; A5-ALERMFLSFP-B5; A1-ALERMFLSFP-B6; A1-ALERMFLSFP-B7; A1-ALERMFLSFP-B8; A1-ALERMFLSFP-B9; A1-ALERMFLSFP-B10.

A1-VADVKDR-B1; A1-VADVKDR-B2; A1-VADVKDR-B3; A1-VADVKDR-B4; A1-VADVKDR-B5; A1-VADVKDR-B6; A1-VADVKDR-B7; A1-VADVKDR-B8; A1-VADVKDR-B9; A1-VADVKDR-B10; A2-VADVKDR-B1; A2-VADVKDR-B2; A2-VADVKDR-B3; A2-VADVKDR-B4; A2-VADVKDR-B5; A1-VADVKDR-B6; A1-VADVKDR-B7; A1-VADVKDR-B8; A1-VADVKDR-B9; A1-VADVKDR-B10; A3-VADVKDR-B1; A3-VADVKDR-B2; A3-VADVKDR-B2; A3-VADVKDR-B3; A3-VADVKDR-B4; A3-VADVKDR-B5; A1-VADVKDR-B6; A1-VADVKDR-B7; A1-VADVKDR-B8; A1-VADVKDR-B9; A1-VADVKDR-B10; A4-VADVKDR-B1; A4-VADVKDR-B2; A4-VADVKDR-B2; A4-VADVKDR-B3; A4-VADVKDR-B4; A4-VADVKDR-B5; A5-VADVKDR-B1; A5-VADVKDR-B2; A5-VADVKDR-B2; A5-VADVKDR-B3; A5-VADVKDR-B4; A5-VADVKDR-B5; A1-VADVKDR-B6; A1-VADVKDR-B7; A1-VADVKDR-B8; A1-VADVKDR-B9; A1-VADVKDR-B10.

A1-L2p-YDAKGD-B1; A1-L2p-YDAKGD-B2; A1-L2p-YDAKGD-B3; A1-L2p-YDAKGD-B4; A1-L2p-YDAKGD-B5; A1-L2p-YDAKGD-B6; A1-L2p-YDAKGD-B7; A1-L2p-YDAKGD-B8; A1-L2p-YDAKGD-B9; A1-L2p-YDAKGD-B10; A2-L2p-YDAKGD-B1; A2-L2P-YDAKGD-B2; A2-L2p-YDAKGD-B3; A2-L2p-YDAKGD-B4; A2-L2p-YDAKGD-B5; A1-L2p-YDAKGD-B6; A1-L2p-YDAKGD-B7; A1-L2p-YDAKGD-B8; A1-L2p-YDAKGD-B9; A1-L2p-YDAKGD-B10; A3-L2p-YDAKGD-B1; A3-L2p-YDAKGD-B2; A3-L2p-YDAKGD-B2; A3-L2p-YDAKGD-B3; A3-L2p-YDAKGD-B4; A3-L2p-YDAKGD-B5; A1-L2p-YDAKGD-B6; A1-L2p-YDAKGD-B7; A1-L2p-YDAKGD-B8; A1-L2p-YDAKGD-B9; A1-L2p-YDAKGD-B10; A4-L2p-YDAKGD-B1; A4-L2p-YDAKGD-B2; A4-L2p-YDAKGD-B2; A4-L2p-YDAKGD-B3; A4-L2p-YDAKGD-B4; A4-L2p-YDAKGD-B5; A5-L2p-YDAKGD-B1; A5-L2p-YDAKGD-B2; A5-L2p-YDAKGD-B2; A5-L2p-YDAKGD-B3; A5-L2p-YDAKGD-B4; A5-L2p-YDAKGD-B5; A1-L2p-YDAKGD-B6; A1-L2p-YDAKGD-B7; A1-L2p-YDAKGD-B8; A1-L2p-YDAKGD-B9; A1-L2p-YDAKGD-B10.

A1-APAKFFRL-B1; A1-APAKFFRL-B2; A1-APAKFFRL-B3; A1-APAKFFRL-B4; A1-APAKFFRL-B5; A1-APAKFFRL-B6; A1-APAKFFRL-B7; A1-APAKFFRL-B8; A1-APAKFFRL-B9; A1-APAKFFRL-B10; A2-APAKFFRL-B1; A2-APAKFFRL-B2; A2-APAKFFRL-B3; A2-APAKFFRL-B4; A2-APAKFFRL-B5; A1-APAKFFRL-B6; A1-APAKFFRL-B7; A1-APAKFFRL-B8; A1-APAKFFRL-B9; A1-APAKFFRL-B10; A3-APAKFFRL-B1; A3-APAKFFRL-B2; A3-APAKFFRL-B2; A3-APAKFFRL-B3; A3-APAKFFRL-B4; A3-APAKFFRL-B5; A1-APAKFFRL-B6; A1-APAKFFRL-B7; A1-APAKFFRL-B8; A1-APAKFFRL-B9; A1-APAKFFRL-B10; A4-APAKFFRL-B1; A4-APAKFFRL-B2; A4-APAKFFRL-B2; A4-APAKFFRL-B3; A4-APAKFFRL-B4; A4-APAKFFRL-B5; A5-APAKFFRL-B1; A5-APAKFFRL-B2; A5-APAKFFRL-B2; A5-APAKFFRL-B3; A5-APAKFFRL-B4; A5-APAKFFRL-B5; A1-APAKFFRL-B6; A1-APAKFFRL-B7; A1-APAKFFRL-B8; A1-APAKFFRL-B9; A1-APAKFFRL-B10.

A1-STPVSSANMK-B1; A1-STPVSSANMK-B2; A1-STPVSSANMK-B3; A1-STPVSSANMK-B4; A1-STPVSSANMK-B5; A1-STPVSSANMK-B6; A1-STPVSSANMK-B7; A1-STPVSSANMK-B8; A1-STPVSSANMK-B9; A1-STPVSSANMK-B10; A2-STPVSSANMK-B1; A2-STPVSSANMK-B2; A2-STPVSSANMK-B3; A2-STPVSSANMK-B4; A2-STPVSSANMK-B5; A1-STPVSSANMK-B6; A1-STPVSSANMK-B7; A1-STPVSSANMK-B8; A1-STPVSSANMK-B9; A1-STPVSSANMK-B10; A3-STPVSSANMK-B1; A3-STPVSSANMK-B2; A3-STPVSSANMK-B2; A3-STPVSSANMK-B3; A3-STPVSSANMK-B4; A3-STPVSSANMK-B5; A1-STPVSSANMK-B6; A1-STPVSSANMK-B7; A1-STPVSSANMK-B8; A1-STPVSSANMK-B9; A1-STPVSSANMK-B10; A4-STPVSSANMK-B1; A4-STPVSSANMK-B2; A4-STPVSSANMK-B2; A4-STPVSSANMK-B3; A4-STPVSSANMK-B4; A4-STPVSSANMK-B5; A5-STPVSSANMK-B1; A5-STPVSSANMK-B2; A5-STPVSSANMK-B2; A5-STPVSSANMK-B3; A5-STPVSSANMK-B4; A5-STPVSSANMK-B5; A1-STPVSSANMK-B6; A1-STPVSSANMK-B7; A1-STPVSSANMK-B8; A1-STPVSSANMK-B9; A1-STPVSSANMK-B10.

A1-EDAEPYAAK-B1; A1-EDAEPYAAK-B2; A1-EDAEPYAAK-B3; A1-EDAEPYAAK-B4; A1-EDAEPYAAK-B5; A1-EDAEPYAAK-B6; A1-EDAEPYAAK-B7; A1-EDAEPYAAK-B8; A1-EDAEPYAAK-B9; A1-EDAEPYAAK-B10; A2-EDAEPYAAK-B1; A2-EDAEPYAAK-B2; A2-EDAEPYAAK-B3; A2-EDAEPYAAK-B4; A2-EDAEPYAAK-B5; A1-EDAEPYAAK-B6; A1-EDAEPYAAK-B7; A1-EDAEPYAAK-B8; A1-EDAEPYAAK-B9; A1-EDAEPYAAK-B10; A3-EDAEPYAAK-B1; A3-EDAEPYAAK-B2; A3-EDAEPYAAK-B2; A3-EDAEPYAAK-B3; A3-EDAEPYAAK-B4; A3-EDAEPYAAK-B5; A1-EDAEPYAAK-B6; A1-EDAEPYAAK-B7; A1-EDAEPYAAK-B8; A1-EDAEPYAAK-B9; A1-EDAEPYAAK-B10; A4-EDAEPYAAK-B1; A4-EDAEPYAAK-B2; A4-EDAEPYAAK-B2; A4-EDAEPYAAK-B3; A4-EDAEPYAAK-B4; A4-EDAEPYAAK-B5; A5-EDAEPYAAK-B1; A5-EDAEPYAAK-B2; A5-EDAEPYAAK-B2; A5-EDAEPYAAK-B3; A5-EDAEPYAAK-B4; A5-EDAEPYAAK-B5; A1-EDAEPYAAK-B6; A1-EDAEPYAAK-B7; A1-EDAEPYAAK-B8; A1-EDAEPYAAK-B9; A1-EDAEPYAAK-B10.

A1-IHPFHLVIHT-B1; A1-IHPFHLVIHT-B2; A1-IHPFHLVIHT-B3; A1-IHPFHLVIHT-B4; A1-IHPFHLVIHT-B5; A1-IHPFHLVIHT-B6; A1-IHPFHLVIHT-B7; A1-IHPFHLVIHT-B8; A1-IHPFHLVIHT-B9; A1-IHPFHLVIHT-B10; A2-IHPFHLVIHT-B1; A2-IHPFHLVIHT-B2; A2-IHPFHLVIHT-B3; A2-IHPFHLVIHT-B4; A2-IHPFHLVIHT-B5; A1-IHPFHLVIHT-B6; A1-IHPFHLVIHT-B7; A1-IHPFHLVIHT-B8; A1-IHPFHLVIHT-B9; A1-IHPFHLVIHT-B10; A3-IHPFHLVIHT-B1; A3-IHPFHLVIHT-B2; A3-IHPFHLVIHT-B2; A3-IHPFHLVIHT-B3; A3-IHPFHLVIHT-B4; A3-IHPFHLVIHT-B5; A1-IHPFHLVIHT-B6; A1-IHPFHLVIHT-B7; A1-IHPFHLVIHT-B8; A1-IHPFHLVIHT-B9; A1-IHPFHLVIHT-B10; A4-IHPFHLVIHT-B1; A4-IHPFHLVIHT-B2; A4-IHPFHLVIHT-B2; A4-IHPFHLVIHT-B3; A4-IHPFHLVIHT-B4; A4-IHPFHLVIHT-B5; A5-IHPFHLVIHT-B1; A5-IHPFHLVIHT-B2; A5-IHPFHLVIHT-B2; A5-IHPFHLVIHT-B3; A5-IHPFHLVIHT-B4; A5-IHPFHLVIHT-B5; A1-IHPFHLVIHT-B6; A1-IHPFHLVIHT-B7; A1-IHPFHLVIHT-B8; A1-IHPFHLVIHT-B9; A1-IHPFHLVIHT-B10.

A1-SVTLQSG-B1; A1-SVTLQSG-B2; A1-SVTLQSG-B3; A1-SVTLQSG-B4; A1-SVTLQSG-B5; A1-SVTLQSG-B6; A1-SVTLQSG-B7; A1-SVTLQSG-B8; A1-SVTLQSG-B9; A1-SVTLQSG-B10; A2-SVTLQSG-B1; A2-SVTLQSG-B2; A2-SVTLQSG-B3; A2-SVTLQSG-B4; A2-SVTLQSG-B5; A1-SVTLQSG-B6; A1-SVTLQSG-B7; A1-SVTLQSG-B8; A1-SVTLQSG-B9; A1-SVTLQSG-B10; A3-SVTLQSG-B1; A3-SVTLQSG-B2; A3-SVTLQSG-B2; A3-SVTLQSG-B3; A3-SVTLQSG-B4; A3-SVTLQSG-B5; A1-SVTLQSG-B6; A1-SVTLQSG-B7; A1-SVTLQSG-B8; A1-SVTLQSG-B9; A1-SVTLQSG-B10; A4-SVTLQSG-B1; A4-SVTLQSG-B2; A4-SVTLQSG-B2; A4-SVTLQSG-B3; A4-SVTLQSG-B4; A4-SVTLQSG-B5; A5-SVTLQSG-B1; A5-SVTLQSG-B2; A5-SVTLQSG-B2; A5-SVTLQSG-B3; A5-SVTLQSG-B4; A5-SVTLQSG-B5; A1-SVTLQSG-B6; A1-SVTLQSG-B7; A1-SVTLQSG-B8; A1-SVTLQSG-B9; A1-SVTLQSG-B10.

A1-TSAVLQSGFR-B1; A1-TSAVLQSGFR-B2; A1-TSAVLQSGFR-B3; A1-TSAVLQSGFR-B4; A1-TSAVLQSGFR-B5; A1-TSAVLQSGFR-B6; A1-TSAVLQSGFR-B7; A1-TSAVLQSGFR-B8; A1-TSAVLQSGFR-B9; A1-TSAVLQSGFR-B10; A2-TSAVLQSGFR-B1; A2-TSAVLQSGFR-B2; A2-TSAVLQSGFR-B3; A2-TSAVLQSGFR-B4; A2-TSAVLQSGFR-B5; A1-TSAVLQSGFR-B6; A1-TSAVLQSGFR-B7; A1-TSAVLQSGFR-B8; A1-TSAVLQSGFR-B9; A1-TSAVLQSGFR-B10; A3-TSAVLQSGFR-B1; A3-TSAVLQSGFR-B2; A3-TSAVLQSGFR-B2; A3-TSAVLQSGFR-B3; A3-TSAVLQSGFR-B4; A3-TSAVLQSGFR-B5; A1-TSAVLQSGFR-B6; A1-TSAVLQSGFR-B7; A1-TSAVLQSGFR-B8; A1-TSAVLQSGFR-B9; A1-TSAVLQSGFR-B10; A4-TSAVLQSGFR-B1; A4-TSAVLQSGFR-B2; A4-TSAVLQSGFR-B2; A4-TSAVLQSGFR-B3; A4-TSAVLQSGFR-B4; A4-TSAVLQSGFR-B5; A5-TSAVLQSGFR-B1; A5-TSAVLQSGFR-B2; A5-TSAVLQSGFR-B2; A5-TSAVLQSGFR-B3; A5-TSAVLQSGFR-B4; A5-TSAVLQSGFR-B5; A1-TSAVLQSGFR-B6; A1-TSAVLQSGFR-B7; A1-TSAVLQSGFR-B8; A1-TSAVLQSGFR-B9; A1-TSAVLQSGFR-B10.

A1-FRLKGGAPIKGV-B1; A1-FRLKGGAPIKGV-B2; A1-FRLKGGAPIKGV-B3; A1-FRLKGGAPIKGV-B4; A1-FRLKGGAPIKGV-B5; A1-FRLKGGAPIKGV-B6; A1-FRLKGGAPIKGV-B7; A1-FRLKGGAPIKGV-B8; A1-FRLKGGAPIKGV-B9; A1-FRLKGGAPIKGV-B10; A2-FRLKGGAPIKGV-B1; A2-FRLKGGAPIKGV-B2; A2-FRLKGGAPIKGV-B3; A2-FRLKGGAPIKGV-B4; A2-FRLKGGAPIKGV-B5; A1-FRLKGGAPIKGV-B6; A1-FRLKGGAPIKGV-B7; A1-FRLKGGAPIKGV-B8; A1-FRLKGGAPIKGV-B9; A1-FRLKGGAPIKGV-B10; A3-FRLKGGAPIKGV-B1; A3-FRLKGGAPIKGV-B2; A3-FRLKGGAPIKGV-B2; A3-FRLKGGAPIKGV-B3; A3-FRLKGGAPIKGV-B4; A3-FRLKGGAPIKGV-B5; A1-FRLKGGAPIKGV-B6; A1-FRLKGGAPIKGV-B7; A1-FRLKGGAPIKGV-B8; A1-FRLKGGAPIKGV-B9; A1-FRLKGGAPIKGV-B10; A4-FRLKGGAPIKGV-B1; A4-FRLKGGAPIKGV-B2; A4-FRLKGGAPIKGV-B2; A4-FRLKGGAPIKGV-B3; A4-FRLKGGAPIKGV-B4; A4-FRLKGGAPIKGV-B5; A5-FRLKGGAPIKGV-B1; A5-FRLKGGAPIKGV-B2; A5-FRLKGGAPIKGV-B2; A5-FRLKGGAPIKGV-B3; A5-FRLKGGAPIKGV-B4; A5-FRLKGGAPIKGV-B5; A1-FRLKGGAPIKGV-B6; A1-FRLKGGAPIKGV-B7; A1-FRLKGGAPIKGV-B8; A1-FRLKGGAPIKGV-B9; A1-FRLKGGAPIKGV-B10.

A1-SPLAQAVRSSSR-B1; A1-SPLAQAVRSSSR-B2; A1-SPLAQAVRSSSR-B3; A1-SPLAQAVRSSSR-B4; A1-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A2-SPLAQAVRSSSR-B1; A2-SPLAQAVRSSSR-B2; A2-SPLAQAVRSSSR-B3; A2-SPLAQAVRSSSR-B4; A2-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A3-SPLAQAVRSSSR-B1; A3-SPLAQAVRSSSR-B2; A3-SPLAQAVRSSSR-B2; A3-SPLAQAVRSSSR-B3; A3-SPLAQAVRSSSR-B4; A3-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A4-SPLAQAVRSSSR-B1; A4-SPLAQAVRSSSR-B2; A4-SPLAQAVRSSSR-B2; A4-SPLAQAVRSSSR-B3; A4-SPLAQAVRSSSR-B4; A4-SPLAQAVRSSSR-B5; A5-SPLAQAVRSSSR-B1; A5-SPLAQAVRSSSR-B2; A5-SPLAQAVRSSSR-B2; A5-SPLAQAVRSSSR-B3; A5-SPLAQAVRSSSR-B4; A5-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10.

A1-DYVPRGSKD-B1; A1-DYVPRGSKD-B2; A1-DYVPRGSKD-B3; A1-DYVPRGSKD-B4; A1-DYVPRGSKD-B5; A1-DYVPRGSKD-B6; A1-DYVPRGSKD-B7; A1-DYVPRGSKD-B8; A1-DYVPRGSKD-B9; A1-DYVPRGSKD-B10; A2-DYVPRGSKD-B1; A2-DYVPRGSKD-B2; A2-DYVPRGSKD-B3; A2-DYVPRGSKD-B4; A2-DYVPRGSKD-B5; A1-DYVPRGSKD-B6; A1-DYVPRGSKD-B7; A1-DYVPRGSKD-B8; A1-DYVPRGSKD-B9; A1-DYVPRGSKD-B10; A3-DYVPRGSKD-B1; A3-DYVPRGSKD-B2; A3-DYVPRGSKD-B2; A3-DYVPRGSKD-B3; A3-DYVPRGSKD-B4; A3-DYVPRGSKD-B5; A1-DYVPRGSKD-B6; A1-DYVPRGSKD-B7; A1-DYVPRGSKD-B8; A1-DYVPRGSKD-B9; A1-DYVPRGSKD-B10; A4-DYVPRGSKD-B1; A4-DYVPRGSKD-B2; A4-DYVPRGSKD-B2; A4-DYVPRGSKD-B3; A4-DYVPRGSKD-B4; A4-DYVPRGSKD-B5; A5-DYVPRGSKD-B1; A5-DYVPRGSKD-B2; A5-DYVPRGSKD-B2; A5-DYVPRGSKD-B3; A5-DYVPRGSKD-B4; A5-DYVPRGSKD-B5; A1-DYVPRGSKD-B6; A1-DYVPRGSKD-B7; A1-DYVPRGSKD-B8; A1-DYVPRGSKD-B9; A1-DYVPRGSKD-B10.

A1-IEPRSFSQ-B1; A1-IEPRSFSQ-B2; A1-IEPRSFSQ-B3; A1-IEPRSFSQ-B4; A1-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10; A2-IEPRSFSQ-B1; A2-IEPRSFSQ-B2; A2-IEPRSFSQ-B3; A2-IEPRSFSQ-B4; A2-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10; A3-IEPRSFSQ-B1; A3-IEPRSFSQ-B2; A3-IEPRSFSQ-B2; A3-IEPRSFSQ-B3; A3-IEPRSFSQ-B4; A3-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10; A4-IEPRSFSQ-B1; A4-IEPRSFSQ-B2; A4-IEPRSFSQ-B2; A4-IEPRSFSQ-B3; A4-IEPRSFSQ-B4; A4-IEPRSFSQ-B5; A5-IEPRSFSQ-B1; A5-IEPRSFSQ-B2; A5-IEPRSFSQ-B2; A5-IEPRSFSQ-B3; A5-IEPRSFSQ-B4; A5-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10.

A1-GVPRSFRG-B1; A1-GVPRSFRG-B2; A1-GVPRSFRG-B3; A1-GVPRSFRG-B4; A1-GVPRSFRG-B5; A1-GVPRSFRG-B6; A1-GVPRSFRG-B7; A1-GVPRSFRG-B8; A1-GVPRSFRG-B9; A1-GVPRSFRG-B10; A2-GVPRSFRG-B1; A2-GVPRSFRG-B2; A2-GVPRSFRG-B3; A2-GVPRSFRG-B4; A2-GVPRSFRG-B5; A1-GVPRSFRG-B6; A1-GVPRSFRG-B7; A1-GVPRSFRG-B8; A1-GVPRSFRG-B9; A1-GVPRSFRG-B10; A3-GVPRSFRG-B1; A3-GVPRSFRG-B2; A3-GVPRSFRG-B2; A3-GVPRSFRG-B3; A3-GVPRSFRG-B4; A3-GVPRSFRG-B5; A1-GVPRSFRG-B6; A1-GVPRSFRG-B7; A1-GVPRSFRG-B8; A1-GVPRSFRG-B9; A1-GVPRSFRG-B10; A4-GVPRSFRG-B1; A4-GVPRSFRG-B2; A4-GVPRSFRG-B2; A4-GVPRSFRG-B3; A4-GVPRSFRG-B4; A4-GVPRSFRG-B5; A5-GVPRSFRG-B1; A5-GVPRSFRG-B2; A5-GVPRSFRG-B2; A5-GVPRSFRG-B3; A5-GVPRSFRG-B4; A5-GVPRSFRG-B5; A1-GVPRSFRG-B6; A1-GVPRSFRG-B7; A1-GVPRSFRG-B8; A1-GVPRSFRG-B9; A1-GVPRSFRG-B10.

A1-ENLYFQSGTK-B1; A1-ENLYFQSGTK-B2; A1-ENLYFQSGTK-B3; A1-ENLYFQSGTK-B4; A1-ENLYFQSGTK-B5; A1-ENLYFQSGTK-B6; A1-ENLYFQSGTK-B7; A1-ENLYFQSGTK-B8; A1-ENLYFQSGTK-B9; A1-ENLYFQSGTK-B10; A2-ENLYFQSGTK-B1; A2-ENLYFQSGTK-B2; A2-ENLYFQSGTK-B3; A2-ENLYFQSGTK-B4; A2-ENLYFQSGTK-B5; A1-ENLYFQSGTK-B6; A1-ENLYFQSGTK-B7; A1-ENLYFQSGTK-B8; A1-ENLYFQSGTK-B9; A1-ENLYFQSGTK-B10; A3-ENLYFQSGTK-B1; A3-ENLYFQSGTK-B2; A3-ENLYFQSGTK-B2; A3-ENLYFQSGTK-B3; A3-ENLYFQSGTK-B4; A3-ENLYFQSGTK-B5; A1-ENLYFQSGTK-B6; A1-ENLYFQSGTK-B7; A1-ENLYFQSGTK-B8; A1-ENLYFQSGTK-B9; A1-ENLYFQSGTK-B10; A4-ENLYFQSGTK-B1; A4-ENLYFQSGTK-B2; A4-ENLYFQSGTK-B2; A4-ENLYFQSGTK-B3; A4-ENLYFQSGTK-B4; A4-ENLYFQSGTK-B5; A5-ENLYFQSGTK-B1; A5-ENLYFQSGTK-B2; A5-ENLYFQSGTK-B2; A5-ENLYFQSGTK-B3; A5-ENLYFQSGTK-B4; A5-ENLYFQSGTK-B5; A1-ENLYFQSGTK-B6; A1-ENLYFQSGTK-B7; A1-ENLYFQSGTK-B8; A1-ENLYFQSGTK-B9; A1-ENLYFQSGTK-B10.

A1-LYFQSGTV-B1; A1-LYFQSGTV-B2; A1-LYFQSGTV-B3; A1-LYFQSGTV-B4; A1-LYFQSGTV-B5; A1-LYFQSGTV-B6; A1-LYFQSGTV-B7; A1-LYFQSGTV-B8; A1-LYFQSGTV-B9; A1-LYFQSGTV-B10; A2-LYFQSGTV-B1; A2-LYFQSGTV-B2; A2-LYFQSGTV-B3; A2-LYFQSGTV-B4; A2-LYFQSGTV-B5; A1-LYFQSGTV-B6; A1-LYFQSGTV-B7; A1-LYFQSGTV-B8; A1-LYFQSGTV-B9; A1-LYFQSGTV-B10; A3-LYFQSGTV-B1; A3-LYFQSGTV-B2; A3-LYFQSGTV-B2; A3-LYFQSGTV-B3; A3-LYFQSGTV-B4; A3-LYFQSGTV-B5; A1-LYFQSGTV-B6; A1-LYFQSGTV-B7; A1-LYFQSGTV-B8; A1-LYFQSGTV-B9; A1-LYFQSGTV-B10; A4-LYFQSGTV-B1; A4-LYFQSGTV-B2; A4-LYFQSGTV-B2; A4-LYFQSGTV-B3; A4-LYFQSGTV-B4; A4-LYFQSGTV-B5; A5-LYFQSGTV-B1; A5-LYFQSGTV-B2; A5-LYFQSGTV-B2; A5-LYFQSGTV-B3; A5-LYFQSGTV-B4; A5-LYFQSGTV-B5; A1-LYFQSGTV-B6; A1-LYFQSGTV-B7; A1-LYFQSGTV-B8; A1-LYFQSGTV-B9; A1-LYFQSGTV-B10.

A1-GRGHARLV-B1; A1-GRGHARLV-B2; A1-GRGHARLV-B3; A1-GRGHARLV-B4; A1-GRGHARLV-B5; A1-GRGHARLV-B6; A1-GRGHARLV-B7; A1-GRGHARLV-B8; A1-GRGHARLV-B9; A1-GRGHARLV-B10; A2-GRGHARLV-B1; A2-GRGHARLV-B2; A2-GRGHARLV-B3; A2-GRGHARLV-B4; A2-GRGHARLV-B5; A1-GRGHARLV-B6; A1-GRGHARLV-B7; A1-GRGHARLV-B8; A1-GRGHARLV-B9; A1-GRGHARLV-B10; A3-GRGHARLV-B1; A3-GRGHARLV-B2; A3-GRGHARLV-B2; A3-GRGHARLV-B3; A3-GRGHARLV-B4; A3-GRGHARLV-B5; A1-GRGHARLV-B6; A1-GRGHARLV-B7; A1-GRGHARLV-B8; A1-GRGHARLV-B9; A1-GRGHARLV-B10; A4-GRGHARLV-B1; A4-GRGHARLV-B2; A4-GRGHARLV-B2; A4-GRGHARLV-B3; A4-GRGHARLV-B4; A4-GRGHARLV-B5; A5-GRGHARLV-B1; A5-GRGHARLV-B2; A5-GRGHARLV-B2; A5-GRGHARLV-B3; A5-GRGHARLV-B4; A5-GRGHARLV-B5; A1-GRGHARLV-B6; A1-GRGHARLV-B7; A1-GRGHARLV-B8; A1-GRGHARLV-B9; A1-GRGHARLV-B10.

A1-GHARLVHG-B1; A1-GHARLVHG-B2; A1-GHARLVHG-B3; A1-GHARLVHG-B4; A1-GHARLVHG-B5; A1-GHARLVHG-B6; A1-GHARLVHG-B7; A1-GHARLVHG-B8; A1-GHARLVHG-B9; A1-GHARLVHG-B10; A2-GHARLVHG-B1; A2-GHARLVHG-B2; A2-GHARLVHG-B3; A2-GHARLVHG-B4; A2-GHARLVHG-B5; A1-GHARLVHG-B6; A1-GHARLVHG-B7; A1-GHARLVHG-B8; A1-GHARLVHG-B9; A1-GHARLVHG-B10; A3-GHARLVHG-B1; A3-GHARLVHG-B2; A3-GHARLVHG-B2; A3-GHARLVHG-B3; A3-GHARLVHG-B4; A3-GHARLVHG-B5; A1-GHARLVHG-B6; A1-GHARLVHG-B7; A1-GHARLVHG-B8; A1-GHARLVHG-B9; A1-GHARLVHG-B10; A4-GHARLVHG-B1; A4-GHARLVHG-B2; A4-GHARLVHG-B2; A4-GHARLVHG-B3; A4-GHARLVHG-B4; A4-GHARLVHG-B5; A5-GHARLVHG-B1; A5-GHARLVHG-B2; A5-GHARLVHG-B2; A5-GHARLVHG-B3; A5-GHARLVHG-B4; A5-GHARLVHG-B5; A1-GHARLVHG-B6; A1-GHARLVHG-B7; A1-GHARLVHG-B8; A1-GHARLVHG-B9; A1-GHARLVHG-B10.

A1-GPAKLAIG-B1; A1-GPAKLAIG-B2; A1-GPAKLAIG-B3; A1-GPAKLAIG-B4; A1-GPAKLAIG-B5; A1-GPAKLAIG-B6; A1-GPAKLAIG-B7; A1-GPAKLAIG-B8; A1-GPAKLAIG-B9; A1-GPAKLAIG-B10; A2-GPAKLAIG-B1; A2-GPAKLAIG-B2; A2-GPAKLAIG-B3; A2-GPAKLAIG-B4; A2-GPAKLAIG-B5; A1-GPAKLAIG-B6; A1-GPAKLAIG-B7; A1-GPAKLAIG-B8; A1-GPAKLAIG-B9; A1-GPAKLAIG-B10; A3-GPAKLAIG-B1; A3-GPAKLAIG-B2; A3-GPAKLAIG-B2; A3-GPAKLAIG-B3; A3-GPAKLAIG-B4; A3-GPAKLAIG-B5; A1-GPAKLAIG-B6; A1-GPAKLAIG-B7; A1-GPAKLAIG-B8; A1-GPAKLAIG-B9; A1-GPAKLAIG-B10; A4-GPAKLAIG-B1; A4-GPAKLAIG-B2; A4-GPAKLAIG-B2; A4-GPAKLAIG-B3; A4-GPAKLAIG-B4; A4-GPAKLAIG-B5; A5-GPAKLAIG-B1; A5-GPAKLAIG-B2; A5-GPAKLAIG-B2; A5-GPAKLAIG-B3; A5-GPAKLAIG-B4; A5-GPAKLAIG-B5; A1-GPAKLAIG-B6; A1-GPAKLAIG-B7; A1-GPAKLAIG-B8; A1-GPAKLAIG-B9; A1-GPAKLAIG-B10.

A1-FASGKRSQIGL-B1; A1-FASGKRSQIGL-B2; A1-FASGKRSQIGL-B3; A1-FASGKRSQIGL-B4; A1-FASGKRSQIGL-B5; A1-FASGKRSQIGL-B6; A1-FASGKRSQIGL-B7; A1-FASGKRSQIGL-B8; A1-FASGKRSQIGL-B9; A1-FASGKRSQIGL-B10; A2-FASGKRSQIGL-B1; A2-FASGKRSQIGL-B2; A2-FASGKRSQIGL-B3; A2-FASGKRSQIGL-B4; A2-FASGKRSQIGL-B5; A1-FASGKRSQIGL-B6; A1-FASGKRSQIGL-B7; A1-FASGKRSQIGL-B8; A1-FASGKRSQIGL-B9; A1-FASGKRSQIGL-B10; A3-FASGKRSQIGL-B1; A3-FASGKRSQIGL-B2; A3-FASGKRSQIGL-B2; A3-FASGKRSQIGL-B3; A3-FASGKRSQIGL-B4; A3-FASGKRSQIGL-B5; A1-FASGKRSQIGL-B6; A1-FASGKRSQIGL-B7; A1-FASGKRSQIGL-B8; A1-FASGKRSQIGL-B9; A1-FASGKRSQIGL-B10; A4-FASGKRSQIGL-B1; A4-FASGKRSQIGL-B2; A4-FASGKRSQIGL-B2; A4-FASGKRSQIGL-B3; A4-FASGKRSQIGL-B4; A4-FASGKRSQIGL-B5; A5-FASGKRSQIGL-B1; A5-FASGKRSQIGL-B2; A5-FASGKRSQIGL-B2; A5-FASGKRSQIGL-B3; A5-FASGKRSQIGL-B4; A5-FASGKRSQIGL-B5; A1-FASGKRSQIGL-B6; A1-FASGKRSQIGL-B7; A1-FASGKRSQIGL-B8; A1-FASGKRSQIGL-B9; A1-FASGKRSQIGL-B10.

The substrates above are used in a method to detect specific protease activity. The method includes contacting a solution containing one or more different proteins with a solution containing a substrate.

The following is one method to measure Cathepsin D activity, although the steps are generalized for measuring other protease activity: Cells (1×10⁶) are collected by centrifugation. The cells are lysed in 200 μl of chilled lysis buffer and incubated on ice for 10 min. The medium is centrifuged 5 min. at top speed, and the clear cell lysate is transferred to a new tube. 5-50 μl of the cell lysate (or approx. 1-10 ng of purified Cathepsin D protein samples) is added into each well of a 96-well plate. The total volume of each well is brought to 50 μl with lysis buffer. A master assay mix is prepared for each assay, including 50 μl reaction buffer and 2 μl of substrate. The master assay mix is combined well, and 52 μl of the master assay mix is added into each assay well. After mixing the assay wells, the plate is incubated at 37° C. for 1 to 2 hours. Samples are read in a fluorometer equipped with a suitable excitation and emission filters.

Kits according to the invention are protease assay kits. The kits include at least one protease substrate listed above. Optionally, the kits can include one or more of the following components: a target enzyme; lysis buffer; and a reaction buffer. One example of such a kit would include 25 ml cell lysis buffer, 5 ml reaction buffer and 0.2 ml (1 mM) protease substrate. The kit could be used according to the general method for measuring Cathepsin D activity discussed above.

The FRET substrates and moieties discussed above are constructed using standard methods known to one of ordinary skill in the art.

1. A protease substrate selected from a group of protease substrates consisting of:
A1-IEPRSFSQ-B1; A1-IEPRSFSQ-B2; A1-IEPRSFSQ-B3; A1-IEPRSFSQ-B4; A1-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10; A2-IEPRSFSQ-B1; A2-IEPRSFSQ-B2; A2-IEPRSFSQ-B3; A2-IEPRSFSQ-B4; A2-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10; A3-IEPRSFSQ-B1; A3-IEPRSFSQ-B2; A3-IEPRSFSQ-B2; A3-IEPRSFSQ-B3; A3-IEPRSFSQ-B4; A3-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10; A4-IEPRSFSQ-B1; A4-IEPRSFSQ-B2; A4-IEPRSFSQ-B2; A4-IEPRSFSQ-B3; A4-IEPRSFSQ-B4; A4-IEPRSFSQ-B5; A5-IEPRSFSQ-B1; A5-IEPRSFSQ-B2; A5-IEPRSFSQ-B2; A5-IEPRSFSQ-B3; A5-IEPRSFSQ-B4; A5-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10; A1-LDRRGIQR-B1; A1-LDRRGIQR-B2; A1-LDRRGIQR-B3; A1-LDRRGIQR-B4; A1-LDRRGIQR-B5; A1-LDRRGIQR-B6; A1-LDRRGIQR-B7; A1-LDRRGIQR-B8; A1-LDRRGIQR-B9; A1-LDRRGIQR-B10; A2-LDRRGIQR-B1; A2-LDRRGIQR-B2; A2-LDRRGIQR-B3; A2-LDRRGIQR-B4; A2-LDRRGIQR-B5; A1-LDRRGIQR-B6; A1-LDRRGIQR-B7; A1-LDRRGIQR-B8; A1-LDRRGIQR-B9; A1-LDRRGIQR-B10; A3-LDRRGIQR-B1; A3-LDRRGIQR-B2; A3-LDRRGIQR-B2; A3-LDRRGIQR-B3; A3-LDRRGIQR-B4; A3-LDRRGIQR-B5; A1-LDRRGIQR-B6; A1-LDRRGIQR-B7; A1-LDRRGIQR-B8; A1-LDRRGIQR-B9; A1-LDRRGIQR-B10; A4-LDRRGIQR-B1; A4-LDRRGIQR-B2; A4-LDRRGIQR-B2; A4-LDRRGIQR-B3; A4-LDRRGIQR-B4; A4-LDRRGIQR-B5; A5-IEPRSFSQ-B1; A5-IEPRSFSQ-B2; A5-IEPRSFSQ-B2; A5-IEPRSFSQ-B3; A5-IEPRSFSQ-B4; A5-IEPRSFSQ-B5; A1-IEPRSFSQ-B6; A1-IEPRSFSQ-B7; A1-IEPRSFSQ-B8; A1-IEPRSFSQ-B9; A1-IEPRSFSQ-B10; A1-AELQGRPISIA-B1; A1-AELQGRPISIA-B2; A1-AELQGRPISIA-B3; A1-AELQGRPISIA-B4; A1-AELQGRPISIA-B5; A1-AELQGRPISIA-B6; A1-AELQGRPISIA-B7; A1-AELQGRPISIA-B8; A1-AELQGRPISIA-B9; A1-AELQGRPISIA-B10; A2-AELQGRPISIA-B1; A2-AELQGRPISIA-B2; A2-AELQGRPISIA-B3; A2-AELQGRPISIA-B4; A2-AELQGRPISIA-B5; A1-AELQGRPISIA-B6; A1-AELQGRPISIA-B7; A1-AELQGRPISIA-B8; A1-AELQGRPISIA-B9; A1-AELQGRPISIA-B10; A3-AELQGRPISIA-B1; A3-AELQGRPISIA-B2; A3-AELQGRPISIA-B2; A3-AELQGRPISIA-B3; A3-AELQGRPISIA-B4; A3-AELQGRPISIA-B5; A1-AELQGRPISIA-B6; A1-AELQGRPISIA-B7; A1-AELQGRPISIA-B8; A1-AELQGRPISIA-B9; A1-AELQGRPISIA-B10; A4-AELQGRPISIA-B1; A4-AELQGRPISIA-B2; A4-AELQGRPISIA-B2; A4-AELQGRPISIA-B3; A4-AELQGRPISIA-B4; A4-AELQGRPISIA-B5; A5-AELQGRPISIA-B1; A5-AELQGRPISIA-B2; A5-AELQGRPISIA-B2; A5-AELQGRPISIA-B3; A5-AELQGRPISIA-B4; A5-AELQGRPISIA-B5; A1-AELQGRPISIA-B6; A1-AELQGRPISIA-B7; A1-AELQGRPISIA-B8; A1-AELQGRPISIA-B9; A1-AELQGRPISIA-B10.

2. A protease substrate selected from a group of protease substrates consisting of:
A1-TEGEARGSVI-B1; A1-TEGEARGSVI-B2; A1-TEGEARGSVI-B3; A1-TEGEARGSVI-B4; A1-TEGEARGSVI-B5; A1-TEGEARGSVI-B6; A1-TEGEARGSVI-B7; A1-TEGEARGSVI-B8; A1-TEGEARGSVI-B9; A1-TEGEARGSVI-B10; A2-TEGEARGSVI-B1; A2-TEGEARGSVI-B2; A2-TEGEARGSVI-B3; A2-TEGEARGSVI-B4; A2-TEGEARGSVI-B5; A1-TEGEARGSVI-B6; A1-TEGEARGSVI-B7; A1-TEGEARGSVI-B8; A1-TEGEARGSVI-B9; A1-TEGEARGSVI-B10; A3-TEGEARGSVI-B1; A3-TEGEARGSVI-B2; A3-TEGEARGSVI-B2; A3-TEGEARGSVI-B3; A3-TEGEARGSVI-B4; A3-TEGEARGSVI-B5; A1-TEGEARGSVI-B6; A1-TEGEARGSVI-B7; A1-TEGEARGSVI-B8; A1-TEGEARGSVI-B9; A1-TEGEARGSVI-B10; A4-TEGEARGSVI-B1; A4-TEGEARGSVI-B2; A4-TEGEARGSVI-B2; A4-TEGEARGSVI-B3; A4-TEGEARGSVI-B4; A4-TEGEARGSVI-B5; A5-TEGEARGSVI-B1; A5-TEGEARGSVI-B2; A5-TEGEARGSVI-B2; A5-TEGEARGSVI-B3; A5-TEGEARGSVI-B4; A5-TEGEARGSVI-B5; A1-TEGEARGSVI-B6; A1-TEGEARGSVI-B7; A1-TEGEARGSVI-B8; A1-TEGEARGSVI-B9; A1-TEGEARGSVI-B10; A1-TESESRGAIY-B1; A1-TESESRGAIY-B2; A1-TESESRGAIY-B3; A1-TESESRGAIY-B4; A1-TESESRGAIY-B5; A1-TESESRGAIY-B6; A1-TESESRGAIY-B7; A1-TESESRGAIY-B8; A1-TESESRGAIY-B9; A1-TESESRGAIY-B10; A2-TESESRGAIY-B1; A2-TESESRGAIY-B2; A2-TESESRGAIY-B3; A2-TESESRGAIY-B4; A2-TESESRGAIY-B5; A1-TESESRGAIY-B6; A1-TESESRGAIY-B7; A1-TESESRGAIY-B8; A1-TESESRGAIY-B9; A1-TESESRGAIY-B10; A3-TESESRGAIY-B1; A3-TESESRGAIY-B2; A3-TESESRGAIY-B2; A3-TESESRGAIY-B3; A3-TESESRGAIY-B4; A3-TESESRGAIY-B5; A1-TESESRGAIY-B6; A1-TESESRGAIY-B7; A1-TESESRGAIY-B8; A1-TESESRGAIY-B9; A1-TESESRGAIY-B10; A4-TESESRGAIY-B1; A4-TESESRGAIY-B2; A4-TESESRGAIY-B2; A4-TESESRGAIY-B3; A4-TESESRGAIY-B4; A4-TESESRGAIY-B5; A5-TESESRGAIY-B1; A5-TESESRGAIY-B2; A5-TESESRGAIY-B2; A5-TESESRGAIY-B3; A5-TESESRGAIY-B4; A5-TESESRGAIY-B5; A1-TESESRGAIY-B6; A1-TESESRGAIY-B7; A1-TESESRGAIY-B8; A1-TESESRGAIY-B9; A1-TESESRGAIY-B10; A1-SPLAQAVRSSSR-B1; A1-SPLAQAVRSSSR-B2; A1-SPLAQAVRSSSR-B3; A1-SPLAQAVRSSSR-B4; A1-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A2-SPLAQAVRSSSR-B1; A2-SPLAQAVRSSSR-B2; A2-SPLAQAVRSSSR-B3; A2-SPLAQAVRSSSR-B4; A2-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A3-SPLAQAVRSSSR-B1; A3-SPLAQAVRSSSR-B2; A3-SPLAQAVRSSSR-B2; A3-SPLAQAVRSSSR-B3; A3-SPLAQAVRSSSR-B4; A3-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A4-SPLAQAVRSSSR-B1; A4-SPLAQAVRSSSR-B2; A4-SPLAQAVRSSSR-B2; A4-SPLAQAVRSSSR-B3; A4-SPLAQAVRSSSR-B4; A4-SPLAQAVRSSSR-B5; A5-SPLAQAVRSSSR-B1; A5-SPLAQAVRSSSR-B2; A5-SPLAQAVRSSSR-B2; A5-SPLAQAVRSSSR-B3; A5-SPLAQAVRSSSR-B4; A5-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10.

3. A protease substrate selected from a group of protease substrates consisting of:
A1-WEVAHQKLA-B1; A1-WEVAHQKLA-B2; A1-WEVAHQKLA-B3; A1-WEVAHQKLA-B4; A1-WEVAHQKLA-B5; A1-WEVAHQKLA-B6; A1-WEVAHQKLA-B7; A1-WEVAHQKLA-B8; A1-WEVAHQKLA-B9; A1-WEVAHQKLA-B10; A2-WEVAHQKLA-B1; A2-WEVAHQKLA-B2; A2-WEVAHQKLA-B3; A2-WEVAHQKLA-B4; A2-WEVAHQKLA-B5; A1-WEVAHQKLA-B6; A1-WEVAHQKLA-B7; A1-WEVAHQKLA-B8; A1-WEVAHQKLA-B9; A1-WEVAHQKLA-B10; A3-WEVAHQKLA-B1; A3-WEVAHQKLA-B2; A3-WEVAHQKLA-B2; A3-WEVAHQKLA-B3; A3-WEVAHQKLA-B4; A3-WEVAHQKLA-B5; A1-WEVAHQKLA-B6; A1-WEVAHQKLA-B7; A1-WEVAHQKLA-B8; A1-WEVAHQKLA-B9; A1-WEVAHQKLA-B10; A4-WEVAHQKLA-B1; A4-WEVAHQKLA-B2; A4-WEVAHQKLA-B2; A4-WEVAHQKLA-B3; A4-WEVAHQKLA-B4; A4-WEVAHQKLA-B5; A5-WEVAHQKLA-B1; A5-WEVAHQKLA-B2; A5-WEVAHQKLA-B2; A5-WEVAHQKLA-B3; A5-WEVAHQKLA-B4; A5-WEVAHQKLA-B5; A1-WEVAHQKLA-B6; A1-WEVAHQKLA-B7; A1-WEVAHQKLA-B8; A1-WEVAHQKLA-B9; A1-WEVAHQKLA-B10; A1-SPLAQAVRSSSR-B1; A1-SPLAQAVRSSSR-B2; A1-SPLAQAVRSSSR-B3; A1-SPLAQAVRSSSR-B4; A1-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A2-SPLAQAVRSSSR-B1; A2-SPLAQAVRSSSR-B2; A2-SPLAQAVRSSSR-B3; A2-SPLAQAVRSSSR-B4; A2-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A3-SPLAQAVRSSSR-B1; A3-SPLAQAVRSSSR-B2; A3-SPLAQAVRSSSR-B2; A3-SPLAQAVRSSSR-B3; A3-SPLAQAVRSSSR-B4; A3-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A4-SPLAQAVRSSSR-B1; A4-SPLAQAVRSSSR-B2; A4-SPLAQAVRSSSR-B2; A4-SPLAQAVRSSSR-B3; A4-SPLAQAVRSSSR-B4; A4-SPLAQAVRSSSR-B5; A5-SPLAQAVRSSSR-B1; A5-SPLAQAVRSSSR-B2; A5-SPLAQAVRSSSR-B2; A5-SPLAQAVRSSSR-B3; A5-SPLAQAVRSSSR-B4; A5-SPLAQAVRSSSR-B5; A1-SPLAQAVRSSSR-B6; A1-SPLAQAVRSSSR-B7; A1-SPLAQAVRSSSR-B8; A1-SPLAQAVRSSSR-B9; A1-SPLAQAVRSSSR-B10; A1-LAQFVRSS-B1; A1-LAQFVRSS-B2; A1-LAQFVRSS-B3; A1-LAQFVRSS-B4; A1-LAQFVRSS-B5; A1-LAQFVRSS-B6; A1-LAQFVRSS-B7; A1-LAQFVRSS-B8; A1-LAQFVRSS-B9; A1-LAQFVRSS-B10; A2-LAQFVRSS-B1; A2-LAQFVRSS-B2; A2-LAQFVRSS-B3; A2-LAQFVRSS-B4; A2-LAQFVRSS-B5; A1-LAQFVRSS-B6; A1-LAQFVRSS-B7; A1-LAQFVRSS-B8; A1-LAQFVRSS-B9; A1-LAQFVRSS-B10; A3-LAQFVRSS-B1; A3-LAQFVRSS-B2; A3-LAQFVRSS-B2; A3-LAQFVRSS-B3; A3-LAQFVRSS-B4; A3-LAQFVRSS-B5; A1-LAQFVRSS-B6; A1-LAQFVRSS-B7; A1-LAQFVRSS-B8; A1-LAQFVRSS-B9; A1-LAQFVRSS-B10; A4-LAQFVRSS-B1; A4-LAQFVRSS-B2; A4-LAQFVRSS-B2; A4-LAQFVRSS-B3; A4-LAQFVRSS-B4; A4-LAQFVRSS-B5; A5-LAQFVRSS-B1; A5-LAQFVRSS-B2; A5-LAQFVRSS-B2; A5-LAQFVRSS-B3; A5-LAQFVRSS-B4; A5-LAQFVRSS-B5; A1-LAQFVRSS-B6; A1-LAQFVRSS-B7; A1-LAQFVRSS-B8; A1-LAQFVRSS-B9; A1-LAQFVRSS-B10.

4. A method for performing a FRET-based assay, wherein the method comprises the following steps: contacting a solution comprising at least one protein with a solution comprising a FRET substrate, wherein the FRET substrate is selected from the substrates of claims 1-3 above.

5. A kit for performing a FRET-based assay, wherein the kit comprises a FRET substrate, wherein the FRET substrate is selected from the substrates of claims 1-3 above.
